SEQUENCE LISTING (112 sequences)

SEQ ID NO: 1 - protein, 315 aa, Artificial Sequence
  Description of Artificial Sequence Synthetic polypeptide
GGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAA

SEQ ID NO: 2 - protein, 280 aa, Artificial Sequence
  Description of Artificial Sequence Synthetic polypeptide
GGQGGRGGFGGLGSQGAGGAGQGGAGAAAAAAAAGGDGGSGLGGYGAGRGHGVGLGGAGGAGAASAAAAAGGQGGRGGFGGLGSQGAGGAGQGGAGAAAAAAAAGGDGGSGLGGYGAGRGHGAGLGGAGGAGAASAAAAAGGQGGRGGFGGLGSQGSGGAGQGGSGAAAAAAAAGGDGGSGLGGYGAGRGYGAGLGGAGGAGAASAAAAAGGQGGRGGFGGLGSQGAGGAGQGGSGAAAAAAAAVADGGSGLGGYGAGRGYGAGLGGAGGAGAASAAAAT

SEQ ID NO: 3 - protein, 278 aa, Artificial Sequence
  Description of Artificial Sequence Synthetic polypeptide
GSAPQGAGGPAPQGPSQQGPVSQGPYGPGAAAAAAAAGGYGPGAGQQGPGSQGPGSGGQQGPGSQGPGSGGQQGPGGQGPYGPSAAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAAGGYGPGAGQQGPGSGGQQGPGGQGPYGSGQQGPGGAGQQGPGGQGPYGPGAAAAAAAAA

SEQ ID NO: 4 - protein, 261 aa, Artificial Sequence
  Description of Artificial Sequence Synthetic polypeptide
GGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAGGYGPGAGQQGPGSQGPGSGGQQGPGGQGPYGPSAAAAAAAAGGYGPGAGQQGPGSGGQQGPGGQGPYGSGQQGPGGAGQQGPGGQGPYGGGYGPGAGQQGPGSQGPGSGGQQGPGGQGPYGPSAAAAAAAA

SEQ ID NO: 5 - protein, 258 aa, Artificial Sequence
  Description of Artificial Sequence Synthetic polypeptide
GPGARRQGPGSQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAA

SEQ ID NO: 6 - protein, 257 aa, Artificial Sequence
  Description of Artificial Sequence Synthetic polypeptide
GPGARRQGPGSQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAA

SEQ ID NO: 7 - protein, 255 aa, Artificial Sequence
  Description of Artificial Sequence Synthetic polypeptide
GGYGPGAGQQGPGSGGQQGPGGQGPYGSGQQGPGGAGQQGPGGQGPYGPGAAAAAAAAAGGYGPGAGQQGPGGAGQQGPEGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAAGGYGPGAGQQGPGSGGQQGPGGQGPYGSGQQGPGGAGQQGPGGQGPYGPGAAAAAAAAA

SEQ ID NO: 8 - protein, 252 aa, Artificial Sequence
  Description of Artificial Sequence Synthetic polypeptide
GVFSAGQGATPWENSQLAESFISRFLRFIGQSGAFSPNQLDDMSSIGDTLKTAIEKMAQSRKSSKSKLQALNMAFASSMAEIAVAEQGGLSLEAKTNAIASALSAAFLETTGYVNQQFVNEIKTLIFMIAQASSNEISGSAAAAGGSSGGGGGSGQGGYGQGAYASASAAAAYGSAPQGTGGPASQGPSQQGPVSQPSYGPSATVAVTAVGGRPQGPSAPRQQGPSQQGPGQQGPGGRGPYGPSAAAAAAAA

SEQ ID NO: 9 - protein, 252 aa, Artificial Sequence
  Description of Artificial Sequence Synthetic polypeptide
GAGAGAGAGAGAGAGAGSGASTSVSTSSSSGSGAGAGAGSGAGSGAGAGSGAGAGAGAGGAGAGFGSGLGLGYGVGLSSAQAQAQAQAAAQAQAQAQAQAYAAAQAQAQAQAQAQAAAAAAAAAAAGAGAGAGAGAGAGAGAGSGASTSVSTSSSSGSGAGAGAGSGAGSGAGAGSGAGAGAGAGGAGAGFGSGLGLGYGVGLSSAQAQAQAQAAAQAQAQAQAQAYAAAQAQAQAQAQAQAAAAAAAAAAA

SEQ ID NO: 10 - protein, 252 aa, Artificial Sequence
  Description of Artificial Sequence Synthetic polypeptide
GAGAGAGAGAGAGAGAGSGASTSVSTSSSSGSGAGAGAGSGAGSGAGAGSGAGAGAGAGGAGAAFGSGLGLGYGVGLSSAQAQAQAQAAAQAQADAQAQAYAAAQAQAQAQAQAQAAAAAAAAAAAGAGAGAGAGSGAGAGAGSGASTSVSTSSSSGSGAGAGAGSGAGSGAGAGSGAGAGAGAGGAGAGFGSGLGLGYGVGLSSAQAQAQAQAAAQAQADAQAQAYAAAQAQAQAQAQAQAAAAAAAAAAA

SEQ ID NO: 11 - protein, 252 aa, Artificial Sequence
  Description of Artificial Sequence Synthetic polypeptide
GAGAGAGAGSGAGAGAGSGASTSVSTSSSSGSGAGAGAGSGAGSGAGAGSGAGAGAGAGGAGAGFGSGLGLGYGVGLSSAQAQAQSAAAARAQADAQAQAYAAAQAQAQAQAQAQAAAAAAAAAAAGAGAGAGAGAGAGAGAGSGASTSVSTSSSSASGAGAGAGSGAGSGAGAGSGAGAGAGAGGAGAGFGSGLGLGYGVGLSSAQAQAQAQAAAQAQAQAQAQALAAAQAQAQAQAQAQAAAATAAAAAA

SEQ ID NO: 12 - protein, 252 aa, Artificial Sequence
  Description of Artificial Sequence Synthetic polypeptide
GGYGPGAGQQGPGGAGQQGPGSQGPGGQGPYGPGAGQQGPGSQGPGSGGQQGPGGQGPYGPSAAAAAAAAGGYGPGAGQQGPGSQGPGSGGQQGPGSQGPGSGGQQGPGGQGPYGPSAAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAA

SEQ ID NO: 13 - protein, 251 aa, Artificial Sequence
  Description of Artificial Sequence Synthetic polypeptide
GGYGPGAGQQGPGGAGQQGPGSQGPGGQGPYGPGAGQQGPGSQGPGSGGQQGPGGQGPYGPSAAAAAAAAGGYGPGAGQQGPGSQGPGSGGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAA

SEQ ID NO: 14 - protein, 248 aa, Artificial Sequence
  Description of Artificial Sequence Synthetic polypeptide
GHQGPHRKTPWETPEMAENFMNNVRENLEASRIFPDELMKDMEAITNTMIAAVDGLEAQHRSSYASLQAMNTAFASSMAQLFATEQDYVDTEVIAGAIGKAYQQITGYENPHLASEVTRLIQLFREEDDLENEVEISFADTDNAIARAAAGAAAGSAAASSSADASATAEGASGDSGFLFSTGTFGRGGAGAGAGAAAASAAAASAAAAGAEGDRGLFFSTGDFGRGGAGAGAGAAAASAAAASAAAA

SEQ ID NO: 15 - protein, 245 aa, Artificial Sequence
  Description of Artificial Sequence Synthetic polypeptide
GGAQKHPSGEYSVATASAAATSVTSGGAPVGKPGVPAPIFYPQGPLQQGPAPGPSNVQPGTSQQGPIGGVGESNTFSSSFASALGGNRGFSGVISSASATAVASAFQKGLAPYGTAFALSAASAAADAYNSIGSGASASAYAQAFARVLYPLLQQYGLSSSADASAFASAIASSFSTGVAGQGPSVPYVGQQQPSIMVSAASASAAASAAAVGGGPVVQGPYDGGQPQQPNIAASAAAAATATSS

SEQ ID NO: 16 - protein, 244 aa, Artificial Sequence
  Description of Artificial Sequence Synthetic polypeptide
GGQGGRGGFGGLGSQGEGGAGQGGAGAAAAAAAAGADGGFGLGGYGAGRGYGAGLGGAGGAGAASAAAAAGGQGGRSGFGGLGSQGAGGAGQGGAGAAAAAAAAGADGGSGLGGYGAGRGYGASLGGADGAGAASAAAAAGGQGGRGGFGGLGSQGAGGAGQGGAGAAAAAAAASGDGGSGLGGYGAGRGYGAGLGGAGGAGAASAAAAAGGEGGRGGFGGLGSQGAGGAGQGGSLAAAAAAAA

SEQ ID NO: 17 - protein, 244 aa, Artificial Sequence
  Description of Artificial Sequence Synthetic polypeptide
GPGGYGGPGQPGPGQGQYGPGPGQQGPRQGGQQGPASAAAAAAAGPGGYGGPGQQGPRQGQQQGPASAAAAAAAAAAGPRGYGGPGQQGPVQGGQQGPASAAAAAAAAGVGGYGGPGQQGPGQGQYGPGTGQQGQGPSGQQGPAGAAAAAAGGAAGPGGYGGPGQQGPGQGQYGPGTGQQGQGPSGQQGPAGAAAAAAAAAGPGGYGGPGQQGPGQGQYGPGAGQQGQGPGSQQGPASAAAAAA

SEQ ID NO: 18 - protein, 243 aa, Artificial Sequence
  Description of Artificial Sequence Synthetic polypeptide
GSGAGQGTGAGAGAAAAAAGAAGSGAGQGAGSGAGAAAAAAAASAAGAGQGAGSGSGAGAAAAAAAAAGAGQGAGSGSGAGAAAAAAAAAAAAQQQQQQQAAAAAAAAAAAAAGSGQGASFGVTQQFGAPSGAASSAAAAAAAAAAAAAGSGAGQEAGTGAGAAAAAAAAGAAGSGAGQGAGSGAGAAAAAAAAASAAGAGQGAGSGSGAGAAAAAAAAAAAAQQQQQQQAAAAAAAAAAAAA

SEQ ID NO: 19 - protein, 242 aa, Artificial Sequence
  Description of Artificial Sequence Synthetic polypeptide
GGAQKQPSGESSVATASAAATSVTSAGAPVGKPGVPAPIFYPQGPLQQGPAPGPSYVQPATSQQGPIGGAGRSNAFSSSFASALSGNRGFSEVISSASATAVASAFQKGLAPYGTAFALSAASAAADAYNSIGSGANAFAYAQAFARVLYPLVQQYGLSSSAKASAFASAIASSFSSGAAGQGQSIPYGGQQQPPMTISAASASAGASAAAVKGGQVGQGPYGGQQQSTAASASAAATTATA

SEQ ID NO: 20 - protein, 241 aa, Artificial Sequence
  Description of Artificial Sequence Synthetic polypeptide
GADGGSGLGGYGAGRGYGAGLGGADGAGAASAAAAAGGQGGRGGFGRLGSQGAGGAGQGGAGAAAAVAAAGGDGGSGLGGYGAGRGYGAGLGGAGGAGAASAAAAAGGQGGRGGFGGLGSQGAGGAGQGGAGAAASGDGGSGLGGYGAGRGYGAGLGGADGAGAASAASAAGGQGGRGGFGGLGSQGAGGAGQGGAGAAAAAATAGGDGGSGLGGYGAGRGYGAGLGGAGGAGAASAAAAA

SEQ ID NO: 21 - protein, 241 aa, Artificial Sequence
  Description of Artificial Sequence Synthetic polypeptide
GAGAGQGGRGGYGQGGFGGQGSGAGAGASAAAGAGAGQGGRGGYGQGGFGGQGSGAGAGASAAAGAGAGQGGRGGYGQGGFGGQGSGAGAGASAAAAAGAGQGGRGGYGQGGLGGSGSGAGAGAGAAAAAAAGAGGYGQGGLGGYGQGAGAGQGGLGGYGSGAGAGASAAAAAGAGGAGQGGLGGYGQGAGAGQGGLGGYGSGAGAGAAAAAAAGAGGSGQGGLGGYGSGGGAGGASAAAA

SEQ ID NO: 22 - protein, 239 aa, Artificial Sequence
  Description of Artificial Sequence Synthetic polypeptide
GAYAYAYAIANAFASILANTGLLSVSSAASVASSVASAIATSVSSSSAAAAASASAAAAASAGASAASSASASSSASAAAGAGAGAGAGASGASGAAGGSGGFGLSSGFGAGIGGLGGYPSGALGGLGIPSGLLSSGLLSPAANQRIASLIPLILSAISPNGVNFGVIGSNIASLASQISQSGGGIAASQAFTQALLELVAAFIQVLSSAQIGAVSSSSASAGATANAFAQSLSSAFAG

SEQ ID NO: 23 - protein, 239 aa, Artificial Sequence
  Description of Artificial Sequence Synthetic polypeptide
GAAQKQPSGESSVATASAAATSVTSGGAPVGKPGVPAPIFYPQGPLQQGPAPGPSNVQPGTSQQGPIGGVGGSNAFSSSFASALSLNRGFTEVISSASATAVASAFQKGLAPYGTAFALSAASAAADAYNSIGSGANAFAYAQAFARVLYPLVRQYGLSSSGKASAFASAIASSFSSGTSGQGPSIGQQQPPVTISAASASAGASAAAVGGGQVGQGPYGGQQQSTAASASAAAATATS

SEQ ID NO: 24 - protein, 239 aa, Artificial Sequence; identical to SEQ ID NO: 23
  Description of Artificial Sequence Synthetic polypeptide

SEQ ID NO: 25 - protein, 239 aa, Artificial Sequence
  Description of Artificial Sequence Synthetic polypeptide
GAAQKQPSGESSVATASAAATSVTSGGAPVGKPGVPAPIFYPQGPLQQGPAPGPSNVQPGTSQQGPIGGVGGSNAFSSSFASALSLNRGFTEVISSASATAVASAFQKGLAPYGTAFALSAASAAADAYNSIGSGANAFAYAQAFARVLYPLVQQYGLSSSAKASAFASAIASSFSSGTSGQGPSIGQQQPPVTISAASASAGASAAAVGGGQVGQGPYGGQQQSTAASASAAAATATS

SEQ ID NO: 26 - protein, 239 aa, Artificial Sequence
  Description of Artificial Sequence Synthetic polypeptide
GGAQKQPSGESSVATASAAATSVTSAGAPVGKPGVPAPIFYPQGPLQQGPAPGPSNVQPGTSQQGPIGGVGGSNAFSSSFASALSLNRGFTEVISSASATAVASAFQKGLAPYGTAFALSAASAAADAYNSIGSGANAFAYAQAFARVLYPLVQQYGLSSSAKASAFASAIASSFSSGTSGQGPSNGQQQPPVTISAASASAGASAAAVGGGQVSQGPYGGQQQSTAASASAAAATATS

SEQ ID NO: 27 - protein, 239 aa, Artificial Sequence
  Description of Artificial Sequence Synthetic polypeptide
GGAQKQPSGESSVATASAAATSVTSAGAPGGKPGVPAPIFYPQGPLQQGPAPGPSNVQPGTSQQGPIGGVGGSNAFSSSFASALSLNRGFTEVISSASATAVASAFQKGLAPYGTAFALSAASAAADAYNSIGSGANAFAYAQAFARVLYPLVQQYGLSSSAKASAFASAIASSFSSGTSGQGPSIGQQQPPVTISAASASAGASAAAVGGGQVGQGPYGGQQQSTAASASAAAATATS

SEQ ID NO: 28 - protein, 236 aa, Artificial Sequence
  Description of Artificial Sequence Synthetic polypeptide
GPGGYGGPGQQGPGQGQQQGPASAAAAAAAAGPGGYGGPGQQGPGQGQQQGPASAAAAAAAAAGPGGYGGPGQQRPGQAQYGRGTGQQGQGPGAQQGPASAAAAAAAGAGLYGGPGQQGPGQGQQQGPASAAAAAAAAAAAGPGGYGGPGQQGPGQAQQQGPASAAAAAAAGPGGYSGPGQQGPGQAQQQGPASAAAAAAAAAGPGGYGGPGQQGPGQGQQQGPASAAAAAAATAA

SEQ ID NO: 29 - protein, 234 aa, Artificial Sequence
  Description of Artificial Sequence Synthetic polypeptide
GAGGDGGLFLSSGDFGRGGAGAGAGAAAASAAAASSAAAGARGGSGFGVGTGGFGRGGAGDGASAAAASAAAASAAAAGAGGDSGLFLSSGDFGRGGAGAGAGAAAASAAAASAAAAGTGGVGGLFLSSGDFGRGGAGAGAGAAAASAAAASSAAAGARGGSGFGVGTGGFGRGGPGAGTGAAAASAAAASAAAAGAGGDSGLFLSSEDFGRGGAGAGTGAAAASAAAASAAAA

SEQ ID NO: 30 - protein, 233 aa, Artificial Sequence
  Description of Artificial Sequence Synthetic polypeptide
GAGRGYGGGYGGGAAAGAGAGAGAGRGYGGGYGGGAGSGAGSGAGAGGGSGYGRGAGAGAGAGAAAAAGAGAGGAGGYGGGAGAGAGASAAAGAGAGAGGAGGYGGGYGGGAGAGAGAGAAAAAGAGAGAGAGRGYGGGFGGGAGSGAGAGAGAGGGSGYGRGAGGYGGGYGGGAGTGAGAAAATGAGAGAGAGRGYGGGYGGGAGAGAGAGAGAGGGSGYGRGAGAGASVAA

SEQ ID NO: 31 - protein, 231 aa, Artificial Sequence
  Description of Artificial Sequence Synthetic polypeptide
GALGQGASVWSSPQMAENFMNGFSMALSQAGAFSGQEMKDFDDVRDIMNSAMDKMIRSGKSGRGAMRAMNAAFGSAIAEIVAANGGKEYQIGAVLDAVTNTLLQLTGNADNGFLNEISRLITLFSSVEANDVSASAGADASGSSGPVGGYSSGAGAAVGQGTAQAVGYGGGAQGVASSAAAGATNYAQGVSTGSTQNVATSTVTTTTNVAGSTATGYNTGYGIGAAAGAAA

SEQ ID NO: 32 - protein, 231 aa, Artificial Sequence
  Description of Artificial Sequence Synthetic polypeptide
GGQGGQGGYDGLGSQGAGQGGYGQGGAAAAAAAASGAGSAQRGGLGAGGAGQGYGAGSGGQGGAGQGGAAAATAAAAGGQGGQGGYGGLGSQGSGQGGYGQGGAAAAAAAASGDGGAGQEGLGAGGAGQGYGAGLGGQGGAGQGGAAAAAAAAAGGQGGQGGYGGLGSQGAGQGGYGQGGAAAAAAAASGAGGAGQGGLGAAGAGQGYGAGSGGQGGAGQGGAAAAAAAAA

SEQ ID NO: 33 - protein, 231 aa, Artificial Sequence
  Description of Artificial Sequence Synthetic polypeptide
GGQGGQGGYGGLGSQGAGQGGYGQGGVAAAAAAASGAGGAGRGGLGAGGAGQEYGAVSGGQGGAGQGGEAAAAAAAAGGQGGQGGYGGLGSQGAGQGGYGQGGAAAAAAAASGAGGARRGGLGAGGAGQGYGAGLGGQGGAGQGSASAAAAAAAGGQGGQGGYGGLGSQGSGQGGYGQGGAAAAAAAASGAGGAGRGSLGAGGAGQGYGAGLGGQGGAGQGGAAAAASAAA

SEQ ID NO: 34 - protein, 229 aa, Artificial Sequence
  Description of Artificial Sequence Synthetic polypeptide
GPGGYGGPGQQGPGQGQYGPGTGQQGQGPGGQQGPVGAAAAAAAAVSSGGYGSQGAGQGGQQGSGQRGPAAAGPGGYSGPGQQGPGQGGQQGPASAAAAAAAAAGPGGYGGSGQQGPGQGRGTGQQGQGPGGQQGPASAAAAAAAGPGGYGGPGQQGPGQGQYGPGTGQQGQGPASAAAAAAAGPGGYGGPGQQGPGQGQYGPGTGQQGQGPGGQQGPGGASAAAAAAA

SEQ ID NO: 35 - protein, 228 aa, Artificial Sequence
  Description of Artificial Sequence Synthetic polypeptide
GGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGPGAGRQGPGSQGPGSGGQQGPGGQGPYGPSAAAAAAAA

SEQ ID NO: 36 - protein, 225 aa, Artificial Sequence
  Description of Artificial Sequence Synthetic polypeptide
GQGGQGGQGGLGQGGYGQGAGSSAAAAAAAAAAAAAAGRGQGGYGQGSGGNAAAAAAAAAAAASGQGSQGGQGGQGQGGYGQGAGSSAAAAAAAAAAAAASGRGQGGYGQGAGGNAAAAAAAAAAAAAAGQGGQGGYGGLGQGGYGQGAGSSAAAAAAAAAAAAGGQGGQGQGGYGQGSGGSAAAAAAAAAAAAAAAGRGQGGYGQGSGGNAAAAAAAAAAAAAA

SEQ ID NO: 37 - protein, 225 aa, Artificial Sequence
  Description of Artificial Sequence Synthetic polypeptide
GRGPGGYGPGQQGPGGPGAAAAAAGPGGYGPGGYGPGQQGPGGPGAAAAAAAGRGPGGYGPGQQGPGQQGPGGSGAAAAAAGRGPGGYGPGQQGPGGPGAAAAAAGPGGYGPGQQGPGAAAAAAAAGRGPGGYGPGQQGPGGPGAAAAAAAGRGPGGYGPGQQGPGQQGPGGSGAAAAAAGRGPGGYGPGQQGPGGPGAAAAAAGPGGYGPGQQGPGAAAAAAAA

SEQ ID NO: 38 - protein, 225 aa, Artificial Sequence
  Description of Artificial Sequence Synthetic polypeptide
GRGPGGYGPGQQGPGGSGAAAAAAGRGPGGYGPGQQGPGGPGAAAAAAGPGGYGPGQQGTGAAAAAAAGSGAGGYGPGQQGPGGPGAAAAAAGPGGYGPGQQGPGAAAAAAAGSGPGGYGPGQQGPGGSSAAAAAAGPGRYGPGQQGPGAAAAASAGRGPGGYGPGQQGPGGPGAAAAAAGPGGYGPGQQGPGAAAAAAAGSGPGGYGPGQQGPGGPGAAAAAAA

SEQ ID NO: 39 - protein, 219 aa, Artificial Sequence
  Description of Artificial Sequence Synthetic polypeptide
GAAATAGAGASVAGGYGGGAGAAAGAGAGGYGGGYGAVAGSGAGAAAAASSGAGGAAGYGRGYGAGSGAGAGAGTVAAYGGAGGVATSSSSATASGSRIVTSGGYGYGTSAAAGAGVAAGSYAGAVNRLSSAEAASRVSSNIAAIASGGASALPSVISNIYSGVVASGVSSNEALIQALLELLSALVHVLSSASIGNVSSVGVDSTLNVVQDSVGQYVG

SEQ ID NO: 40 - protein, 219 aa, Artificial Sequence
  Description of Artificial Sequence Synthetic polypeptide
GGQGGFSGQGQGGFGPGAGSSAAAAAAAAAAARQGGQGQGGFGQGAGGNAAAAAAAAAAAAAAQQGGQGGFSGRGQGGFGPGAGSSAAAAAAGQGGQGQGGFGQGAGGNAAAAAAAAAAAAAAAGQGGQGRGGFGQGAGGNAAAAAAAAAAAAAAAQQGGQGGFGGRGQGGFGPGAGSSAAAAAAGQGGQGRGGFGQGAGGNAAAASAAAAASAAAAGQ

SEQ ID NO: 41 - protein, 218 aa, Artificial Sequence
  Description of Artificial Sequence Synthetic polypeptide
GGYGPGAGQQGPGGAGQQGPGSQGPGGAGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGLGPYGPSAAAAAAAAGGYGPGAGQQGPGSQGPGSGGQQRPGGLGPYGPSAAAAAAAAGGYGPGAGQQGPGSQGPGSGGQQRPGGLGPYGPSAAAAAAAA

SEQ ID NO: 42 - protein, 217 aa, Artificial Sequence
  Description of Artificial Sequence Synthetic polypeptide
GAGAGGGYGGGYSAGGGAGAGSGAAAGAGAGRGGAGGYSAGAGTGAGAAAGAGTAGGYSGGYGAGASSSAGSSFISSSSMSSSQATGYSSSSGYGGGAASAAAGAGAAAGGYGGGYGAGAGAGAAAASGATGRVANSLGAMASGGINALPGVFSNIFSQVSAASGGASGGAVLVQALTEVIALLLHILSSASIGNVSSQGLEGSMAIAQQAIGAYAG

SEQ ID NO: 43 - protein, 216 aa, Artificial Sequence
  Description of Artificial Sequence Synthetic polypeptide
GAGAGGAGGYAQGYGAGAGAGAGAGTGAGGAGGYGQGYGAGSGAGAGGAGGYGAGAGAGAGAGDASGYGQGYGDGAGAGAGAAAAAGAAAGARGAGGYGGGAGAGAGAGAGAAGGYGQGYGAGAGEGAGAGAGAGAVAGAGAAAAAGAGAGAGGAEGYGAGAGAGGAGGYGQSYGDGAAAAAGSGAGAGGSGGYGAGAGAGSGAGAAGGYGGGAGA

SEQ ID NO: 44 - protein, 216 aa, Artificial Sequence
  Description of Artificial Sequence Synthetic polypeptide
GPGGYGPGQQGPGGYGPGQQGPGRYGPGQQGPSGPGSAAAAAAGSGQQGPGGYGPRQQGPGGYGQGQQGPSGPGSAAAASAAASAESGQQGPGGYGPGQQGPGGYGPGQQGPGGYGPGQQGPSGPGSAAAAAAAASGPGQQGPGGYGPGQQGPGGYGPGQQGPSGPGSAAAAAAAASGPGQQGPGGYGPGQQGPGGYGPGQQGLSGPGSAAAAAAA

SEQ ID NO: 45 - protein, 216 aa, Artificial Sequence
  Description of Artificial Sequence Synthetic polypeptide
GRGPGGYGQGQQGPGGPGAAAAAAGPGGYGPGQQGPGAAAAAAAGSGPGGYGPGQQGPGRSGAAAAAAAAGRGPGGYGPGQQGPGGPGAAAAAAGPGGYGPGQQGPGAAAAASAGRGPGGYGPGQQGPGGSGAAAAAAGRGPGGYGPGQQGPGGPGAAAAAAAGRGPGGYGPGQQGPGQQGPGGSGAAAAAAGRGPGGYGPGQQGPGGPGAAAAAA

SEQ ID NO: 46 - protein, 214 aa, Artificial Sequence
  Description of Artificial Sequence Synthetic polypeptide
GVGAGGEGGYDQGYGAGAGAGSGGGAGGAGGYGGGAGAGSGGGAGGAGGYGGGAGAGAGAGAGGAGGYGGGAGAGTGARAGAGGVGGYGQSYGAGASAAAGAGVGAGGAGAGGAGGYGQGYGAGAGIGAGDAGGYGGGAGAGASAGAGGYGGGAGAGAGGVGGYGKGYGAGSGAGAAAAAGAGAGSAGGYGRGDGAGAGGASGYGQGYGAGAAA

SEQ ID NO: 47 - protein, 212 aa, Artificial Sequence
  Description of Artificial Sequence Synthetic polypeptide
GYGAGAGRGYGAGAGAGAGAVAASGAGAGAGYGAGAGAGAGAGYGAGAGRGYGAGAGAGAGSGAASGAGAGAGYGAGAGAGAGYGAGAGSGYGTGAGAGAGAAAAGGAGAGAGYGAGAGRGYGAGAGAGAASGAGAGAGAGAASGAGAGSGYGAGAAAAGGAGAGAGGGYGAGAGRGYGAGAGAGAGAGSGSGSAAGYGQGYGSGSGAGAAA

SEQ ID NO: 48 - protein, 198 aa, Artificial Sequence
  Description of Artificial Sequence Synthetic polypeptide
GQGTDSSASSVSTSTSVSSSATGPDTGYPVGYYGAGQAEAAASAAAAAAASAAEAATIAGLGYGRQGQGTDSSASSVSTSTSVSSSATGPDMGYPVGNYGAGQAEAAASAAAAAAASAAEAATIASLGYGRQGQGTDSSASSVSTSTSVSSSATGPGSRYPVRDYGADQAEAAASAAAAAAAAASAAEEIASLGYGRQ

SEQ ID NO: 49 - protein, 198 aa, Artificial Sequence
  Description of Artificial Sequence Synthetic polypeptide
GQGTDSVASSASSSASASSSATGPDTGYPVGYYGAGQAEAAASAAAAAAASAAEAATIAGLGYGRQGQGTDSSASSVSTSTSVSSSATGPGSRYPVRDYGADQAEAAASATAAAAAAASAAEEIASLGYGRQGQGTDSVASSASSSASASSSATGPDTGYPVGYYGAGQAEAAASAAAAAAASAAEAATIAGLGYGRQ

SEQ ID NO: 50 - protein, 195 aa, Artificial Sequence
  Description of Artificial Sequence Synthetic polypeptide
GQGGQGGYGGLGQGGYGQGAGSSAAAAAAAAAAAAAGGQGGQGQGRYGQGAGSSAAAAAAAAAAAAAAGRGQGGYGQGSGGNAAAAAAAAAAAASGQGSQGGQGGQGQGGYGQGAGSSAAAAAAAAAAAAASGRGQGGYGQGAGGNAAAAAAAAAAAAAAGQGGQGGYGGLGQGGYGQGAGSSAAAAAAAAAAAA

SEQ ID NO: 51 - protein, 193 aa, Artificial Sequence
  Description of Artificial Sequence Synthetic polypeptide
GGLGGQGGLGGLGSQGAGLGGYGQGGAGQGGAAAAAAAAGGLGGQGGRGGLGSQGAGQGGYGQGGAGQGGAAAAAAAAGGLGGQGGLGALGSQGAGQGGAGQGGYGQGGAAAAAAGGLGGQGGLGGLGSQGAGQGGYGQGGAGQGGAAAAAAAAGGLGGQGGLGGLGSQGAGPGGYGQGGAGQGGAAAAAAAA

SEQ ID NO: 52 - protein, 192 aa, Artificial Sequence
  Description of Artificial Sequence Synthetic polypeptide
GGQGRGGFGQGAGGNAAAAAAAAAAAAAAQQVGQFGFGGRGQGGFGPFAGSSAAAAAAASAAAGQGGQGQGGFGQGAGGNAAAAAAAAAAAARQGGQGQGGFSQGAGGNAAAAAAAAAAAAAAAQQGGQGGFGGRGQGGFGPGAGSSAAAAAAATAAAGQGGQGRGGFGQGAGSNAAAAAAAAAAAAAAAGQ

SEQ ID NO: 53 - protein, 190 aa, Artificial Sequence
  Description of Artificial Sequence Synthetic polypeptide
GGQGGQGGYGGLGSQGAGQGGYGAGQGAAAAAAAAGGAGGAGRGGLGAGGAGQGYGAGLGGQGGAGQAAAAAAAGGAGGARQGGLGAGGAGQGYGAGLGGQGGAGQGGAAAAAAAAGGQGGQGGYGGLGSQGAGQGGYGAGQGGAAAAAAAAGGQGGQGGYGGLGSQGAGQGGYGGRQGGAGAAAAAAAA

SEQ ID NO: 54 - protein, 188 aa, Artificial Sequence
  Description of Artificial Sequence Synthetic polypeptide
GGAGQRGYGGLGNQGAGRGGLGGQGAGAAAAAAAGGAGQGGYGGLGNQGAGRGGQGAAAAAGGAGQGGYGGLGSQGAGRGGQGAGAAAAAAVGAGQEGIRGQGAGQGGYGGLGSQGSGRGGLGGQGAGAAAAAAGGAGQGGLGGQGAGQGAGAAAAAAGGVRQGGYGGLGSQGAGRGGQGAGAAAAAA

SEQ ID NO: 55 - protein, 186 aa, Artificial Sequence
  Description of Artificial Sequence Synthetic polypeptide
GGAGQGGLGGQGAGQGAGASAAAAGGAGQGGYGGLGSQGAGRGGEGAGAAAAAAGGAGQGGYGGLGGQGAGQGGYGGLGSQGAGRGGLGGQGAGAAAAGGAGQGGLGGQGAGQGAGAAAAAAGGAGQGGYGGLGSQGAGRGGLGGQGAGAVAAAAAGGAGQGGYGGLGSQGAGRGGQGAGAAAAAA

SEQ ID NO: 56 - protein, 182 aa, Artificial Sequence
  Description of Artificial Sequence Synthetic polypeptide
GAGAGAGAGSGAGAAGGYGGGAGAGVGAGGAGGYDQGYGAGAGAGSGAGAGGAGGYGGGAGAGADAGAGGAGGYGGGAGAGAGARAGAGGVGGYGQSYGAGAGAGAGVGAGGAGAGGADGYGQGYGAGAGTGAGDAGGYGGGAGAGASAGAGGYGGGAGAGGVGVYGKGYGSGSGAGAAAAA

SEQ ID NO: 57 - protein, 182 aa, Artificial Sequence
  Description of Artificial Sequence Synthetic polypeptide
GGAGGYGVGQGYGAGAGAGAAAGAGAGGAGGYGAGQGYGAGAGVGAAAAAGAGAGVGGAGGYGRGAGAGAGAGAGAAAGAGAGAAAGAGAGGAGGYGAGQGYGAGAGVGAAAAAGAGAGVGGAGGYGRGAGAGAGAGAGGAGGYGRGAGAGAGAGAGAGGAGGYGAGQGYGAGAGAGAAAAA

SEQ ID NO: 58 - protein, 182 aa, Artificial Sequence
  Description of Artificial Sequence Synthetic polypeptide
GEAFSASSASSAVVFESAGPGEEAGSSGDGASAAASAAAAAGAGSGRRGPGGARSRGGAGAGAGAGSGVGGYGSGSGAGAGAGAGAGAGGEGGFGEGQGYGAGAGAGFGSGAGAGAGAGSGAGAGEGVGSGAGAGAGAGFGVGAGAGAGAGAGFGSGAGAGSGAGAGYGAGRAGGRGRGGRG

SEQ ID NO: 59 - protein, 182 aa, Artificial Sequence
  Description of Artificial Sequence Synthetic polypeptide
GEAFSASSASSAVVFESAGPGEEAGSSGGGASAAASAAAAAGAGSGRRGPGGARSRGGAGAGAGAGSGVGGYGSGSGAGAGAGAGAGAGGEGGFGEGQGYGAGAGAGFGSGAGAGAGAGSGAGAGEGVGSGAGAGAGAGFGVGAGAGAGAGAGFGSGAGAGSGAGAGYGAGRAGGRGRGGRG

SEQ ID NO: 60 - protein, 182 aa, Artificial Sequence
  Description of Artificial Sequence Synthetic polypeptide
GNGLGQALLANGVLNSGNYLQLANSLAYSFGSSLSQYSSSAAGASAAGAASGAAGAGAGAASSGGSSGSASSSTTTTTTTSTSAAAAAAAAAAAASAAASTSASASASASASASAFSQTFVQTVLQSAAFGSYFGGNLSLQSAQAAASAAAQAAAQQIGLGSYGYALANAVASAFASAGANA

SEQ ID NO: 61 - protein, 182 aa, Artificial Sequence; identical to SEQ ID NO: 60
  Description of Artificial Sequence Synthetic polypeptide

SEQ ID NO: 62 - protein, 182 aa, Artificial Sequence; identical to SEQ ID NO: 60
  Description of Artificial Sequence Synthetic polypeptide

SEQ ID NO: 63 - protein, 180 aa, Artificial Sequence
  Description of Artificial Sequence Synthetic polypeptide
GASGAGQGQGYGQQGQGGSSAAAAAAAAAAAAAAAQGQGQGYGQQGQGSAAAAAAAAAAGASGAGQGQGYGQQGQGSAAAAAAAAAAGASGAGQGQGYGQQGQGGSSAAAAAAAAAAAAAAAAQGQGYGQQGQGSAAAAAAAAAGASGAGQGQGYGQQGQGGSSAAAAAAAAAAAAAAAA

SEQ ID NO: 64 - protein, 179 aa, Artificial Sequence
  Description of Artificial Sequence Synthetic polypeptide
GRGQGGYGQGSGGNAAAAAAAGQGGFGGQEGNGQGAGSAAAAAAAAAAAAGGSGQGRYGGRGQGGYGQGAGAAASAAAAAAAAAAGQGGFGGQEGNGQGAGSAAAAAAAAAAAAGGSGQGGYGGRGQGGYGQGAGAAAAAAAAAAAAAAGQGGQGGFGSQGGNGQGAGSAAAAAAAAAA

SEQ ID NO: 65 - protein, 178 aa, Artificial Sequence
  Description of Artificial Sequence Synthetic polypeptide
GQNTPWSSTELADAFINAFMNEAGRTGAFTADQLDDMSTIGDTIKTAMDKMARSNKSSKGKLQALNMAFASSMAEIAAVEQGGLSVDAKTNAIADSLNSAFYQTTGAANPQFVNEIRSLINMFAQSSANEVSYGGGYGGQSAGAAASAAAAGGGGQGGYGNLGGQGAGAAAAAAASAA

SEQ ID NO: 66 - protein, 178 aa, Artificial Sequence
  Description of Artificial Sequence Synthetic polypeptide
GQNTPWSSTELADAFINAFLNEAGRTGAFTADQLDDMSTIGDTLKTAMDKMARSNKSSQSKLQALNMAFASSMAEIAAVEQGGLSVAEKTNAIADSLNSAFYQTTGAVNVQFVNEIRSLISMFAQASANEVSYGGGYGGGQGGQSAGAAAAAASAGAGQGGYGGLGGQGAGSAAAAAA

SEQ ID NO: 67 - protein, 177 aa, Artificial Sequence
  Description of Artificial Sequence Synthetic polypeptide
GGQGGQGGYGGLGSQGAGQGGYGQGGAAAAAASAGGQGGQGGYGGLGSQGAGQGGYGGGAFSGQQGGAASVATASAAASRLSSPGAASRVSSAVTSLVSSGGPTNSAALSNTISNVVSQISSSNPGLSGCDVLVQALLEIVSALVHILGSANIGQVNSSGVGRSASIVGQSINQAFS

SEQ ID NO: 68 - protein, 177 aa, Artificial Sequence
  Description of Artificial Sequence Synthetic polypeptide
GGAGQGGYGGLGGQGAGAAAAAAGGAGQGGYGGQGAGQGAAAAAASGAGQGGYEGPGAGQGAGAAAAAAGGAGQGGYGGLGGQGAGQGAGAAAAAAGGAGQGGYGGLGGQGAGQGAGAAAAAAGGAGQGGYGGQGAGQGAAAAAAGGAGQGGYGGLGSGQGGYGRQGAGAAAAAAAA

SEQ ID NO: 69 - protein, 175 aa, Artificial Sequence
  Description of Artificial Sequence Synthetic polypeptide
GASSAAAAAAATATSGGAPGGYGGYGPGIGGAFVPASTTGTGSGSGSGAGAAGSGGLGGLGSSGGSGGLGGGNGGSGASAAASAAAASSSPGSGGYGPGQGVGSGSGSGAAGGSGTGSGAGGPGSGGYGGPQFFASAYGGQGLLGTSGYGNGQGGASGTGSGGVGGSGSGAGSNS

SEQ ID NO: 70 - protein, 174 aa, Artificial Sequence
  Description of Artificial Sequence Synthetic polypeptide
GQPIWTNPNAAMTMTNNLVQCASRSGVLTADQMDDMGMMADSVNSQMQKMGPNPPQHRLRAMNTAMAAEVAEVVATSPPQSYSAVLNTIGACLRESMMQATGSVDNAFTNEVMQLVKMLSADSANEVSTASASGASYATSTSSAVSSSQATGYSTAAGYGNAAGAGAGAAAAVS

SEQ ID NO: 71 - protein, 174 aa, Artificial Sequence
  Description of Artificial Sequence Synthetic polypeptide
GQKIWTNPDAAMAMTNNLVQCAGRSGALTADQMDDLGMVSDSVNSQVRKMGANAPPHKIKAMSTAVAAGVAEVVASSPPQSYSAVLNTIGGCLRESMMQVTGSVDNTFTTEMMQMVNMFAADNANEVSASASGSGASYATGTSSAVSTSQATGYSTAGGYGTAAGAGAGAAAAA

SEQ ID NO: 72 - protein, 174 aa, Artificial Sequence
  Description of Artificial Sequence Synthetic polypeptide
GSGYGAGAGAGAGSGYGAGAGAGSGYGAGAGAGAGSGYVAGAGAGAGAGSGYGAGAGAGAGSSYSAGAGAGAGSGYGAGSSASAGSAVSTQTVSSSATTSSQSAAAATGAAYGTRASTGSGASAGAAASGAGAGYGGQAGYGQGGGAAAYRAGAGSQAAYGQGASGSSGAAAAA

SEQ ID NO: 73 - protein, 171 aa, Artificial Sequence
  Description of Artificial Sequence Synthetic polypeptide
GGQGGRGGFGGLSSQGAGGAGQGGSGAAAAAAAAGGDGGSGLGDYGAGRGYGAGLGGAGGAGVASAAASAAASRLSSPSAASRVSSAVTSLISGGGPTNPAALSNTFSNVVYQISVSSPGLSGCDVLIQALLELVSALVHILGSAIIGQVNSSAAGESASLVGQSVYQAFS

SEQ ID NO: 74 - protein, 169 aa, Artificial Sequence
  Description of Artificial Sequence Synthetic polypeptide
GVGQAATPWENSQLAEDFINSFLRFIAQSGAFSPNQLDDMSSIGDTLKTAIEKMAQSRKSSKSKLQALNMAFASSMAEIAVAEQGGLSLEAKTNAIANALASAFLETTGFVNQQFVSEIKSLIYMIAQASSNEISGSAAAAGGGSGGGGGSGQGGYGQGASASASAAAA

SEQ ID NO: 75 - protein, 169 aa, Artificial Sequence
  Description of Artificial Sequence Synthetic polypeptide
GGGDGYGQGGYGNQRGVGSYGQGAGAGAAATSAAGGAGSGRGGYGEQGGLGGYGQGAGAGAASTAAGGGDGYGQGGYGNQGGRGSYGQGSGAGAGAAVAAAAGGAVSGQGGYDGEGGQGGYGQGSGAGAAVAAASGGTGAGQGGYGSQGSQAGYGQGAGFRAAAATAAA

SEQ ID NO: 76 - protein, 168 aa, Artificial Sequence
  Description of Artificial Sequence Synthetic polypeptide
GAGAGYGGQVGYGQGAGASAGAAAAGAGAGYGGQAGYGQGAGGSAGAAAAGAGAGRQAGYGQGAGASARAAAAGAGTGYGQGAGASAGAAAAGAGAGSQVGYGQGAGASSGAAAAAGAGAGYGGQVGYEQGAGASAGAEAAASSAGAGYGGQAGYGQGAGASAGAAAA

SEQ ID NO: 77 - protein, 166 aa, Artificial Sequence
  Description of Artificial Sequence Synthetic polypeptide
GGAGQGGYGGLGGQGAGQGGLGGQRAGAAAAAAGGAGQGGYGGLGSQGAGRGGYGGVGSGASAASAAASRLSSPEASSRVSSAVSNLVSSGPTNSAALSSTISNVVSQISASNPGLSGCDVLVQALLEVVSALIQILGSSSIGQVNYGTAGQAAQIVGQSVYQALG

SEQ ID NO: 78 - protein, 166 aa, Artificial Sequence
  Description of Artificial Sequence Synthetic polypeptide
GGYGPGSGQQGPGGAGQQGPGGQGPYGPGSSSAAAVGGYGPSSGLQGPAGQGPYGPGAAASAAAAAGASRLSSPQASSRVSSAVSSLVSSGPTNSAALTNTISSVVSQISASNPGLSGCDVLIQALLEIVSALVHILGYSSIGQINYDAAAQYASLVGQSVAQALA

SEQ ID NO: 79 - protein, 166 aa, Artificial Sequence
  Description of Artificial Sequence Synthetic polypeptide
GGAGAGQGSYGGQGGYGQGGAGAATATAAAAGGAGSGQGGYGGQGGLGGYGQGAGAGAAAAAAAAAGGAGAGQGGYGGQGGQGGYGQGAGAGAAAAAAGGAGAGQGGYGGQGGYGQGGGAGAAAAAAAASGGSGSGQGGYGGQGGLGGYGQGAGAGAGAAASAAAA

SEQ ID NO: 80 - protein, 165 aa, Artificial Sequence
  Description of Artificial Sequence Synthetic polypeptide
GQGGQGGYGRQSQGAGSAAAAAAAAAAAAAAGSGQGGYGGQGQGGYGQSSASASAAASAASTVANSVSRLSSPSAVSRVSSAVSSLVSNGQVNMAALPNIISNISSSVSASAPGASGCEVIVQALLEVITALVQIVSSSSVGYINPSAVNQITNVVANAMAQVMG

SEQ ID NO: 81 - protein, 164 aa, Artificial Sequence
  Description of Artificial Sequence Synthetic polypeptide
GGAGQGGYGGLGGQGSGAAAAGTGQGGYGSLGGQGAGAAGAAAAAVGGAGQGGYGGVGSAAASAAASRLSSPEASSRVSSAVSNLVSSGPTNSAALSNTISNVVSQISSSNPGLSGCDVLVQALLEVVSALIHILGSSSIGQVNYGSAGQATQIVGQSVYQALG

SEQ ID NO: 82 - protein, 164 aa, Artificial Sequence
  Description of Artificial Sequence Synthetic polypeptide
GAGAGGAGGYGAGQGYGAGAGAGAAAGAGAGGARGYGARQGYGSGAGAGAGARAGGAGGYGRGAGAGAAAASGAGAGGYGAGQGYGAGAGAVASAAAGAGSGAGGAGGYGRGAGAVAGAGAGGAGGYGAGAGAAAGVGAGGSGGYGGRQGGYSAGAGAGAAAAA

SEQ ID NO: 83 - protein, 163 aa, Artificial Sequence
  Description of Artificial Sequence Synthetic polypeptide
GQGGQGGYGGLGQGGYGQGAGSSAAAAAAAAAAAGRGQGGYGQGSGGNAAAAAAAAAAAASGQGGQGGQGGQGQGGYGQGAGSSAAAAAAAAAAAAAAAGRGQGGYGQGAGGNAAAAAAAAAAAASGQGGQGGQGGQGQGGYGQGAGSSAAAAAAAAAAAAAA

SEQ ID NO: 84 - protein, 162 aa, Artificial Sequence
  Description of Artificial Sequence Synthetic polypeptide
GGYGPGSGQQGPGQQGPGQQGPGQQGPYGAGASAAAAAAGGYGPGSGQQGPGVRVAAPVASAAASRLSSSAASSRVSSAVSSLVSSGPTTPAALSNTISSAVSQISASNPGLSGCDVLVQALLEVVSALVHILGSSSVGQINYGASAQYAQMVGQSVTQALV

SEQ ID NO: 85 - protein, 161 aa, Artificial Sequence
  Description of Artificial Sequence Synthetic polypeptide
GAGAGGAGYGRGAGAGAGAAAGAGAGAAAGAGAGAGGYGGQGGYGAGAGAGAAAAAGAGAGGAAGYSRGGRAGAAGAGAGAAAGAGAGAGGYGGQGGYGAGAGAGAAAAAGAGSGGAGGYGRGAGAGAAAGAGAAAGAGAGAGGYGGQGGYGAGAGAAAAA

SEQ ID NO: 86 - protein, 160 aa, Artificial Sequence
  Description of Artificial Sequence Synthetic polypeptide
GAGAGRGGYGRGAGAGGYGGQGGYGAGAGAGAAAAAGAGAGGYGDKEIACWSRCRYTVASTTSRLSSAEASSRISSAASTLVSGGYLNTAALPSVISDLFAQVGASSPGVSDSEVLIQVLLEIVSSLIHILSSSSVGQVDFSSVGSSAAAVGQSMQVVMG

SEQ ID NO: 87 - protein, 160 aa, Artificial Sequence
  Description of Artificial Sequence Synthetic polypeptide
GAGAGAGGAGGYGRGAGAGAGAGAGAAAGQGYGSGAGAGAGASAGGAGSYGRGAGAGAAAASGAGAGGYGAGQGYGAGAGAVASAAAGAGSGAGGAGGYGRGAVAGSGAGAGAGAGGAGGYGAGAGAGAAAGAVAGGSGGYGGRQGGYSAGAGAGAAAAA

SEQ ID NO: 88 - protein, 159 aa, Artificial Sequence
  Description of Artificial Sequence Synthetic polypeptide
GPGGYGPVQQGPSGPGSAAGPGGYGPAQQGPARYGPGSAAAAAAAAGSAGYGPGPQASAAASRLASPDSGARVASAVSNLVSSGPTSSAALSSVISNAVSQIGASNPGLSGCDVLIQALLEIVSACVTILSSSSIGQVNYGAASQFAQVVGQSVLSAFS

SEQ ID NO: 89 - protein, 156 aa, Artificial Sequence
  Description of Artificial Sequence Synthetic polypeptide
GTGGVGGLFLSSGDFGRGGAGAGAGAAAASAAAASSAAAGARGGSGFGVGTGGFGRGGAGAGTGAAAASAAAASAAAAGAGGDGGLFLSSGDFGRGGAGAGAGAAAASAAAASSAAAGARGGSGFGVGTGGFGRGGAGDGASAAAASAAAASAAAA

SEQ ID NO: 90 - protein, 153 aa, Artificial Sequence
  Description of Artificial Sequence Synthetic polypeptide
GGYGPGAGQQGPGGAGQQGPGGQGPYGPSVAAAASAAGGYGPGAGQQGPVASAAVSRLSSPQASSRVSSAVSSLVSSGPTNPAALSNAMSSVVSQVSASNPGLSGCDVLVQALLEIVSALVHILGSSSIGQINYAASSQYAQMVGQSVAQALA

SEQ ID NO: 91 - protein, 153 aa, Artificial Sequence
  Description of Artificial Sequence Synthetic polypeptide
GGAGQGGYGGLGSQGAGRGGYGGQGAGAAAAATGGAGQGGYGGVGSGASAASAAASRLSSPQASSRVSSAVSNLVASGPTNSAALSSTISNAVSQIGASNPGLSGCDVLIQALLEVVSALIHILGSSSIGQVNYGSAGQATQIVGQSVYQALG

SEQ ID NO: 92 - protein, 153 aa, Artificial Sequence
  Description of Artificial Sequence Synthetic polypeptide
GGAGQGGYGGLGSQGAGRGGYGGQGAGAAVAAIGGVGQGGYGGVGSGASAASAAASRLSSPEASSRVSSAVSNLVSSGPTNSAALSSTISNVVSQIGASNPGLSGCDVLIQALLEVVSALVHILGSSSIGQVNYGSAGQATQIVGQSVYQALG

SEQ ID NO: 93 - protein, 152 aa, Artificial Sequence
  Description of Artificial Sequence Synthetic polypeptide
GASGGYGGGAGEGAGAAAAAGAGAGGAGGYGGGAGSGAGAVARAGAGGAGGYGSGIGGGYGSGAGAAAGAGAGGAGAYGGGYGTGAGAGARGADSAGAAAGYGGGVGTGTGSSAGYGRGAGAGAGAGAAAGSGAGAAGGYGGGYGAGAGAGA

SEQ ID NO: 94 - protein, 152 aa, Artificial Sequence
  Description of Artificial Sequence Synthetic polypeptide
GAGSGQGGYGGQGGLGGYGQGAGAGAAAGASGSGSGGAGQGGLGGYGQGAGAGAAAAAAGASGAGQGGFGPYGSSYQSSTSYSVTSQGAAGGLGGYGQGSGAGAAAAGAAGQGGQGGYGQGAGAGAGAGAGQGGLGGYGQGAGSSAASAAAA

SEQ ID NO: 95 - protein, 151 aa, Artificial Sequence
  Description of Artificial Sequence Synthetic polypeptide
GGAGQGGYGGLGGQGVGRGGLGGQGAGAAAAGGAGQGGYGGVGSGASAASAAASRLSSPQASSRLSSAVSNLVATGPTNSAALSSTISNVVSQIGASNPGLSGCDVLIQALLEVVSALIQILGSSSIGQVNYGSAGQATQIVGQSVYQALG

SEQ ID NO: 96 - protein, 150 aa, Artificial Sequence
  Description of Artificial Sequence Synthetic polypeptide
GAGSGGAGGYGRGAGAGAGAAAGAGAGAGSYGGQGGYGAGAGAGAAAAAGAGAGAGGYGRGAGAGAGAGAGAAARAGAGAGGAGYGGQGGYGAGAGAGAAAAAGAGAGGAGGYGRGAGAGAGAAAGAGAGAGGYGGQSGYGAGAGAAAAA

SEQ ID NO: 97 - protein, 150 aa, Artificial Sequence
  Description of Artificial Sequence Synthetic polypeptide
GASGAGQGQGYGQQGQGGSSAAAAAAAAAAAQGQGQGYGQQGQGYGQQGQGGSSAAAAAAAAAAAAAQGQGQGYGQQGQGSAAAAAAAAAGASGAGQGQGYGQQGQGGSSAAAAAAAAAAAAAAAQGQGYGQQGQGSAAAAAAAAAAAAA

SEQ ID NO: 98 - protein, 149 aa, Artificial Sequence
  Description of Artificial Sequence Synthetic polypeptide
GGYGPRYGQQGPGAGPYGPGAGATAAAAGGYGPGAGQQGPRSQAPVASAAAARLSSPQAGSRVSSAVSTLVSSGPTNPASLSNAIGSVVSQVSASNPGLPSCDVLVQALLEIVSALVHILGSSSIGQINYSASSQYARLVGQSIAQALG

SEQ ID NO: 99 - protein, 1800 aa, Artificial Sequence
  Description of Artificial Sequence Synthetic polypeptide
  MISC_FEATURE (7)..(11): This region may encompass “SGGQQ,” “GAGQQ,” “GQGPY,” “AGQQ” or “SQ,” wherein some positions may be absent
  MISC_FEATURE (15)..(19): This region may encompass “SGGQQ,” “GAGQQ,” “GQGPY,” “AGQQ” or “SQ,” wherein some positions may be absent
  MISC_FEATURE (23)..(27): This region may encompass “SGGQQ,” “GAGQQ,” “GQGPY,” “AGQQ” or “SQ,” wherein some positions may be absent
  MISC_FEATURE (31)..(35): This region may encompass “SGGQQ,” “GAGQQ,” “GQGPY,” “AGQQ” or “SQ,” wherein some positions may be absent
  MISC_FEATURE (39)..(43): This region may encompass “SGGQQ,” “GAGQQ,” “GQGPY,” “AGQQ” or “SQ,” wherein some positions may be absent
  MISC_FEATURE (47)..(51): This region may encompass “SGGQQ,” “GAGQQ,” “GQGPY,” “AGQQ” or “SQ,” wherein some positions may be absent
  MISC_FEATURE (55)..(59): This region may encompass “SGGQQ,” “GAGQQ,” “GQGPY,” “AGQQ” or “SQ,” wherein some positions may be absent
  MISC_FEATURE (63)..(67): This region may encompass “SGGQQ,” “GAGQQ,” “GQGPY,” “AGQQ” or “SQ,” wherein some positions may be absent
  MISC_FEATURE (4)..(67): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (71)..(90): This region may encompass 6-20 residues
  MISC_FEATURE (97)..(101): This region may encompass “SGGQQ,” “GAGQQ,” “GQGPY,” “AGQQ” or “SQ,” wherein some positions may be absent
  MISC_FEATURE (105)..(109): This region may encompass “SGGQQ,” “GAGQQ,” “GQGPY,” “AGQQ” or “SQ,” wherein some positions may be absent
  MISC_FEATURE (113)..(117): This region may encompass “SGGQQ,” “GAGQQ,” “GQGPY,” “AGQQ” or “SQ,” wherein some positions may be absent
  MISC_FEATURE (121)..(125): This region may encompass “SGGQQ,” “GAGQQ,” “GQGPY,” “AGQQ” or “SQ,” wherein some positions may be absent
  MISC_FEATURE (129)..(133): This region may encompass “SGGQQ,” “GAGQQ,” “GQGPY,” “AGQQ” or “SQ,” wherein some positions may be absent
  MISC_FEATURE (137)..(141): This region may encompass “SGGQQ,” “GAGQQ,” “GQGPY,” “AGQQ” or “SQ,” wherein some positions may be absent
  MISC_FEATURE (145)..(149): This region may encompass “SGGQQ,” “GAGQQ,” “GQGPY,” “AGQQ” or “SQ,” wherein some positions may be absent
  MISC_FEATURE (153)..(157): This region may encompass “SGGQQ,” “GAGQQ,” “GQGPY,” “AGQQ” or “SQ,” wherein some positions may be absent
  MISC_FEATURE (94)..(157): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (161)..(180): This region may encompass 6-20 residues
  MISC_FEATURE (187)..(191): This region may encompass “SGGQQ,” “GAGQQ,” “GQGPY,” “AGQQ” or “SQ,” wherein some positions may be absent
  MISC_FEATURE (195)..(199): This region may encompass “SGGQQ,” “GAGQQ,” “GQGPY,” “AGQQ” or “SQ,” wherein some positions may be absent
  MISC_FEATURE (203)..(207): This region may encompass “SGGQQ,” “GAGQQ,” “GQGPY,” “AGQQ” or “SQ,” wherein some positions may be absent
  MISC_FEATURE (211)..(215): This region may encompass “SGGQQ,” “GAGQQ,” “GQGPY,” “AGQQ” or “SQ,” wherein some positions may be absent
  MISC_FEATURE (219)..(223): This region may encompass “SGGQQ,” “GAGQQ,” “GQGPY,” “AGQQ” or “SQ,” wherein some positions may be absent
  MISC_FEATURE (227)..(231): This region may encompass “SGGQQ,” “GAGQQ,” “GQGPY,” “AGQQ” or “SQ,” wherein some positions may be absent
  MISC_FEATURE (235)..(239): This region may encompass “SGGQQ,” “GAGQQ,” “GQGPY,” “AGQQ” or “SQ,” wherein some positions may be absent
  MISC_FEATURE (243)..(247): This region may encompass “SGGQQ,” “GAGQQ,” “GQGPY,” “AGQQ” or “SQ,” wherein some positions may be absent
  MISC_FEATURE (184)..(247): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (251)..(270): This region may encompass 6-20 residues
  MISC_FEATURE (277)..(281): This region may encompass “SGGQQ,” “GAGQQ,” “GQGPY,” “AGQQ” or “SQ,” wherein some positions may be absent
  MISC_FEATURE (285)..(289): This region may encompass “SGGQQ,” “GAGQQ,” “GQGPY,” “AGQQ” or “SQ,” wherein some positions may be absent
  MISC_FEATURE (293)..(297): This region may encompass “SGGQQ,” “GAGQQ,” “GQGPY,” “AGQQ” or “SQ,” wherein some positions may be absent
  MISC_FEATURE (301)..(305): This region may encompass “SGGQQ,” “GAGQQ,” “GQGPY,” “AGQQ” or “SQ,” wherein some positions may be absent
  MISC_FEATURE (309)..(313): This region may encompass “SGGQQ,” “GAGQQ,” “GQGPY,” “AGQQ” or “SQ,” wherein some positions may be absent
  MISC_FEATURE (317)..(321): This region may encompass “SGGQQ,” “GAGQQ,” “GQGPY,” “AGQQ” or “SQ,” wherein some positions may be absent
  MISC_FEATURE (325)..(329): This region may encompass “SGGQQ,” “GAGQQ,” “GQGPY,” “AGQQ” or “SQ,” wherein some positions may be absent
  MISC_FEATURE (333)..(337): This region may encompass “SGGQQ,” “GAGQQ,” “GQGPY,” “AGQQ” or “SQ,” wherein some positions may be absent
  MISC_FEATURE (274)..(337): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (341)..(360): This region may encompass 6-20 residues
  MISC_FEATURE (367)..(371): This region may encompass “SGGQQ,” “GAGQQ,” “GQGPY,” “AGQQ” or “SQ,” wherein some positions may be absent
  MISC_FEATURE (375)..(379): This region may encompass “SGGQQ,” “GAGQQ,” “GQGPY,” “AGQQ” or “SQ,” wherein some positions may be absent
  MISC_FEATURE (383)..(387): This region may encompass “SGGQQ,” “GAGQQ,” “GQGPY,” “AGQQ” or “SQ,” wherein some positions may be absent
  MISC_FEATURE (391)..(395): This region may encompass “SGGQQ,” “GAGQQ,” “GQGPY,” “AGQQ” or “SQ,” wherein some positions may be absent
  MISC_FEATURE (399)..(403): This region may encompass “SGGQQ,” “GAGQQ,” “GQGPY,” “AGQQ” or “SQ,” wherein some positions may be absent
  MISC_FEATURE (407)..(411): This region may encompass “SGGQQ,” “GAGQQ,” “GQGPY,” “AGQQ” or “SQ,” wherein some positions may be absent
  MISC_FEATURE (415)..(419): This region may encompass “SGGQQ,” “GAGQQ,” “GQGPY,” “AGQQ” or “SQ,” wherein some positions may be absent
  MISC_FEATURE (423)..(427): This region may encompass “SGGQQ,” “GAGQQ,” “GQGPY,” “AGQQ” or “SQ,” wherein some positions may be absent
  MISC_FEATURE (364)..(427): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (431)..(450): This region may encompass 6-20 residues
  MISC_FEATURE (457)..(461): This region may encompass “SGGQQ,” “GAGQQ,” “GQGPY,” “AGQQ” or “SQ,” wherein some positions may be absent
  MISC_FEATURE (465)..(469): This region may encompass “SGGQQ,” “GAGQQ,” “GQGPY,” “AGQQ” or “SQ,” wherein some positions may be absent
  MISC_FEATURE (473)..(477): This region may encompass “SGGQQ,” “GAGQQ,” “GQGPY,” “AGQQ” or “SQ,” wherein some positions may be absent
  MISC_FEATURE (481)..(485): This region may encompass “SGGQQ,” “GAGQQ,” “GQGPY,” “AGQQ” or “SQ,” wherein some positions may be absent
  MISC_FEATURE (489)..(493): This region may encompass “SGGQQ,” “GAGQQ,” “GQGPY,” “AGQQ” or “SQ,” wherein some positions may be absent
  MISC_FEATURE (497)..(501): This region may encompass “SGGQQ,” “GAGQQ,” “GQGPY,” “AGQQ” or “SQ,” wherein some positions may be absent
  MISC_FEATURE (505)..(509): This region may encompass “SGGQQ,” “GAGQQ,” “GQGPY,” “AGQQ” or “SQ,” wherein some positions may be absent
  MISC_FEATURE (513)..(517): This region may encompass “SGGQQ,” “GAGQQ,” “GQGPY,” “AGQQ” or “SQ,” wherein some positions may be absent
  MISC_FEATURE (454)..(517): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (521)..(540): This region may encompass 6-20 residues
  MISC_FEATURE (547)..(551): This region may encompass “SGGQQ,” “GAGQQ,” “GQGPY,” “AGQQ” or “SQ,” wherein some positions may be absent
  MISC_FEATURE (555)..(559): This region may encompass “SGGQQ,” “GAGQQ,” “GQGPY,” “AGQQ” or “SQ,” wherein some positions may be absent
  MISC_FEATURE (563)..(567): This region may encompass “SGGQQ,” “GAGQQ,” “GQGPY,” “AGQQ” or “SQ,” wherein some positions may be absent
  MISC_FEATURE (571)..(575): This region may encompass “SGGQQ,” “GAGQQ,” “GQGPY,” “AGQQ” or “SQ,” wherein some positions may be absent
  MISC_FEATURE (579)..(583): This region may encompass “SGGQQ,” “GAGQQ,” “GQGPY,” “AGQQ” or “SQ,” wherein some positions may be absent
  MISC_FEATURE (587)..(591): This region may encompass “SGGQQ,” “GAGQQ,” “GQGPY,” “AGQQ” or “SQ,” wherein some positions may be absent
  MISC_FEATURE (595)..(599): This region may encompass “SGGQQ,” “GAGQQ,” “GQGPY,” “AGQQ” or “SQ,” wherein some positions may be absent
  MISC_FEATURE (603)..(607): This region may encompass “SGGQQ,” “GAGQQ,” “GQGPY,” “AGQQ” or “SQ,” wherein some positions may be absent
  MISC_FEATURE (544)..(607): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (611)..(630): This region may encompass 6-20 residues
  MISC_FEATURE (637)..(641): This region may encompass “SGGQQ,” “GAGQQ,” “GQGPY,” “AGQQ” or “SQ,” wherein some positions may be absent
  MISC_FEATURE (645)..(649): This region may encompass “SGGQQ,” “GAGQQ,” “GQGPY,” “AGQQ” or “SQ,” wherein some positions may be absent
  MISC_FEATURE (653)..(657): This region may encompass “SGGQQ,” “GAGQQ,” “GQGPY,” “AGQQ” or “SQ,” wherein some positions may be absent
  MISC_FEATURE (661)..(665): This region may encompass “SGGQQ,” “GAGQQ,” “GQGPY,” “AGQQ” or “SQ,” wherein some positions may be absent
  MISC_FEATURE (669)..(673): This region may encompass “SGGQQ,” “GAGQQ,” “GQGPY,” “AGQQ” or “SQ,” wherein some positions may be absent
  MISC_FEATURE (677)..(681): This region may encompass “SGGQQ,” “GAGQQ,” “GQGPY,” “AGQQ” or “SQ,” wherein some positions may be absent
  MISC_FEATURE (685)..(689): This region may encompass “SGGQQ,” “GAGQQ,” “GQGPY,” “AGQQ” or “SQ,” wherein some positions may be absent
  MISC_FEATURE (693)..(697): This region may encompass “SGGQQ,” “GAGQQ,” “GQGPY,” “AGQQ” or “SQ,” wherein some positions may be absent
  MISC_FEATURE (634)..(697): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (701)..(720): This region may encompass 6-20 residues
  MISC_FEATURE (727)..(731): This region may encompass “SGGQQ,” “GAGQQ,” “GQGPY,” “AGQQ” or “SQ,” wherein some positions may be absent
  MISC_FEATURE (735)..(739): This region may encompass “SGGQQ,” “GAGQQ,” “GQGPY,” “AGQQ” or “SQ,” wherein some positions may be absent
  MISC_FEATURE (743)..(747): This region may encompass “SGGQQ,” “GAGQQ,” “GQGPY,” “AGQQ” or “SQ,” wherein some positions may be absent
  MISC_FEATURE (751)..(755): This region may encompass “SGGQQ,” “GAGQQ,” “GQGPY,” “AGQQ” or “SQ,” wherein some positions may be absent
  MISC_FEATURE (759)..(763): This region may encompass “SGGQQ,” “GAGQQ,” “GQGPY,” “AGQQ” or “SQ,” wherein some positions may be absent
  MISC_FEATURE (767)..(771): This region may encompass “SGGQQ,” “GAGQQ,” “GQGPY,” “AGQQ” or “SQ,” wherein some positions may be absent
  MISC_FEATURE (775)..(779): This region may encompass “SGGQQ,” “GAGQQ,” “GQGPY,” “AGQQ” or “SQ,” wherein some positions may be absent
  MISC_FEATURE (783)..(787): This region may encompass “SGGQQ,” “GAGQQ,” “GQGPY,” “AGQQ” or “SQ,” wherein some positions may be absent
  MISC_FEATURE (724)..(787): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (791)..(810): This region may encompass 6-20 residues
  MISC_FEATURE (817)..(821): This region may encompass “SGGQQ,” “GAGQQ,” “GQGPY,” “AGQQ” or “SQ,” wherein some positions may be absent
  MISC_FEATURE (825)..(829): This region may encompass “SGGQQ,” “GAGQQ,” “GQGPY,” “AGQQ” or “SQ,” wherein some positions may be absent
  MISC_FEATURE (833)..(837): This region may encompass “SGGQQ,” “GAGQQ,” “GQGPY,” “AGQQ” or “SQ,” wherein some positions may be absent
  MISC_FEATURE (841)..(845): This region may encompass “SGGQQ,” “GAGQQ,” “GQGPY,” “AGQQ” or “SQ,” wherein some positions may be absent
  MISC_FEATURE (849)..(853): This region may encompass “SGGQQ,” “GAGQQ,” “GQGPY,” “AGQQ” or “SQ,” wherein some positions may be absent
  MISC_FEATURE (857)..(861): This region may encompass “SGGQQ,” “GAGQQ,” “GQGPY,” “AGQQ” or “SQ,” wherein some positions may be absent
  MISC_FEATURE (865)..(869): This region may encompass “SGGQQ,” “GAGQQ,” “GQGPY,” “AGQQ” or “SQ,” wherein some positions may be absent
  MISC_FEATURE (873)..(877): This region may encompass “SGGQQ,” “GAGQQ,” “GQGPY,” “AGQQ” or “SQ,” wherein some positions may be absent
  MISC_FEATURE (814)..(877): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (881)..(900): This region may encompass 6-20 residues
  MISC_FEATURE (907)..(911): This region may encompass “SGGQQ,” “GAGQQ,” “GQGPY,” “AGQQ” or “SQ,” wherein some positions may be absent
  MISC_FEATURE (915)..(919): This region may encompass “SGGQQ,” “GAGQQ,” “GQGPY,” “AGQQ” or “SQ,” wherein some positions may be absent
  MISC_FEATURE (923)..(927): This region may encompass “SGGQQ,” “GAGQQ,” “GQGPY,” “AGQQ” or “SQ,” wherein some positions may be absent
  MISC_FEATURE (931)..(935): This region may encompass “SGGQQ,” “GAGQQ,” “GQGPY,” “AGQQ” or “SQ,” wherein some positions may be absent
  MISC_FEATURE (939)..(943): This region may encompass “SGGQQ,” “GAGQQ,” “GQGPY,” “AGQQ” or “SQ,” wherein some positions may be absent
  MISC_FEATURE (947)..(951): This region may encompass “SGGQQ,” “GAGQQ,” “GQGPY,” “AGQQ” or “SQ,” wherein some positions may be absent
  MISC_FEATURE (955)..(959): This region may encompass “SGGQQ,” “GAGQQ,” “GQGPY,” “AGQQ” or “SQ,” wherein some positions may be absent
  MISC_FEATURE (963)..(967): This region may encompass “SGGQQ,” “GAGQQ,” “GQGPY,” “AGQQ” or “SQ,” wherein some positions may be absent
  MISC_FEATURE (904)..(967): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (971)..(990): This region may encompass 6-20 residues
  MISC_FEATURE (997)..(1001): This region may encompass “SGGQQ,” “GAGQQ,” “GQGPY,” “AGQQ” or “SQ,” wherein some positions may be absent
  MISC_FEATURE (1005)..(1009): This region may encompass “SGGQQ,” “GAGQQ,” “GQGPY,” “AGQQ” or “SQ,” wherein some positions may be absent
  MISC_FEATURE (1013)..(1017): This region may encompass “SGGQQ,” “GAGQQ,” “GQGPY,” “AGQQ” or “SQ,” wherein some positions may be absent
  MISC_FEATURE (1021)..(1025): This region may encompass “SGGQQ,” “GAGQQ,” “GQGPY,” “AGQQ” or “SQ,” wherein some positions may be absent
  MISC_FEATURE (1029)..(1033): This region may encompass “SGGQQ,” “GAGQQ,” “GQGPY,” “AGQQ” or “SQ,” wherein some positions may be absent
  MISC_FEATURE (1037)..(1041): This region may encompass “SGGQQ,” “GAGQQ,” “GQGPY,” “AGQQ” or “SQ,” wherein some positions may be absent
  MISC_FEATURE (1045)..(1049): This region may encompass “SGGQQ,” “GAGQQ,” “GQGPY,” “AGQQ” or “SQ,” wherein some positions may be absent
  MISC_FEATURE (1053)..(1057): This region may encompass “SGGQQ,” “GAGQQ,” “GQGPY,” “AGQQ” or “SQ,” wherein some positions may be absent
  MISC_FEATURE (994)..(1057): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (1061)..(1080): This region may encompass 6-20 residues
  MISC_FEATURE (1087)..(1091): This region may encompass “SGGQQ,” “GAGQQ,” “GQGPY,” “AGQQ” or “SQ,” wherein some positions may be absent
  MISC_FEATURE (1095)..(1099): This region may encompass “SGGQQ,” “GAGQQ,” “GQGPY,” “AGQQ” or “SQ,” wherein some positions may be absent
  MISC_FEATURE (1103)..(1107): This region may encompass “SGGQQ,” “GAGQQ,” “GQGPY,” “AGQQ” or “SQ,” wherein some positions may be absent
  MISC_FEATURE (1111)..(1115): This region may encompass “SGGQQ,” “GAGQQ,” “GQGPY,” “AGQQ” or “SQ,” wherein some positions may be absent
  MISC_FEATURE (1119)..(1123): This region may encompass “SGGQQ,” “GAGQQ,” “GQGPY,” “AGQQ” or “SQ,” wherein some positions may be absent
  MISC_FEATURE (1127)..(1131): This region may encompass “SGGQQ,” “GAGQQ,” “GQGPY,” “AGQQ” or “SQ,” wherein some positions may be absent
  MISC_FEATURE (1135)..(1139): This region may encompass “SGGQQ,” “GAGQQ,” “GQGPY,” “AGQQ” or “SQ,” wherein some positions may be absent
  MISC_FEATURE (1143)..(1147): This region may encompass “SGGQQ,” “GAGQQ,” “GQGPY,” “AGQQ” or “SQ,” wherein some positions may be absent
  MISC_FEATURE (1084)..(1147): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (1151)..(1170): This region may encompass 6-20 residues
  MISC_FEATURE (1177)..(1181): This region may encompass “SGGQQ,” “GAGQQ,” “GQGPY,” “AGQQ” or “SQ,” wherein some positions may be absent
  MISC_FEATURE (1185)..(1189): This region may encompass “SGGQQ,” “GAGQQ,” “GQGPY,” “AGQQ” or “SQ,” wherein some positions may be absent
  MISC_FEATURE (1193)..(1197): This region may encompass “SGGQQ,” “GAGQQ,” “GQGPY,” “AGQQ” or “SQ,” wherein some positions may be absent
  MISC_FEATURE (1201)..(1205): This region may encompass “SGGQQ,” “GAGQQ,” “GQGPY,” “AGQQ” or “SQ,” wherein some positions may be absent
  MISC_FEATURE (1209)..(1213): This region may encompass “SGGQQ,” “GAGQQ,” “GQGPY,” “AGQQ” or “SQ,” wherein some positions may be absent
  MISC_FEATURE (1217)..(1221): This region may encompass “SGGQQ,” “GAGQQ,” “GQGPY,” “AGQQ” or “SQ,” wherein some positions may be absent
  MISC_FEATURE (1225)..(1229): This region may encompass “SGGQQ,” “GAGQQ,” “GQGPY,” “AGQQ” or “SQ,” wherein some positions may be absent
  MISC_FEATURE (1233)..(1237): This region may encompass “SGGQQ,” “GAGQQ,” “GQGPY,” “AGQQ” or “SQ,” wherein some positions may be absent
  MISC_FEATURE (1174)..(1237): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (1241)..(1260): This region may encompass 6-20 residues
  MISC_FEATURE (1267)..(1271): This region may encompass “SGGQQ,” “GAGQQ,” “GQGPY,” “AGQQ” or “SQ,” wherein some positions may be absent
  MISC_FEATURE (1275)..(1279): This region may encompass “SGGQQ,” “GAGQQ,” “GQGPY,” “AGQQ” or “SQ,” wherein some positions may be absent
  MISC_FEATURE (1283)..(1287): This region may encompass “SGGQQ,” “GAGQQ,” “GQGPY,” “AGQQ” or “SQ,” wherein some positions may be absent
  MISC_FEATURE (1291)..(1295): This region may encompass “SGGQQ,” “GAGQQ,” “GQGPY,” “AGQQ” or “SQ,” wherein some positions may be absent
  MISC_FEATURE (1299)..(1303): This region may encompass “SGGQQ,” “GAGQQ,” “GQGPY,” “AGQQ” or “SQ,” wherein some positions may be absent
  MISC_FEATURE (1307)..(1311): This region may encompass “SGGQQ,” “GAGQQ,” “GQGPY,” “AGQQ” or “SQ,” wherein some positions may be absent
  MISC_FEATURE (1315)..(1319): This region may encompass “SGGQQ,” “GAGQQ,” “GQGPY,” “AGQQ” or “SQ,” wherein some positions may be absent
  MISC_FEATURE (1323)..(1327): This region may encompass “SGGQQ,” “GAGQQ,” “GQGPY,” “AGQQ” or “SQ,” wherein some positions may be absent
  MISC_FEATURE (1264)..(1327): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (1331)..(1350): This region may encompass 6-20 residues
  MISC_FEATURE (1357)..(1361): This region may encompass “SGGQQ,” “GAGQQ,” “GQGPY,” “AGQQ” or “SQ,” wherein some positions may be absent
  MISC_FEATURE (1365)..(1369): This region may encompass “SGGQQ,” “GAGQQ,” “GQGPY,” “AGQQ” or “SQ,” wherein some positions may be absent
  MISC_FEATURE (1373)..(1377): This region may encompass “SGGQQ,” “GAGQQ,” “GQGPY,” “AGQQ” or “SQ,” wherein some positions may be absent
  MISC_FEATURE (1381)..(1385): This region may encompass “SGGQQ,” “GAGQQ,” “GQGPY,” “AGQQ” or “SQ,” wherein some positions may be absent
  MISC_FEATURE (1389)..(1393): This region may encompass “SGGQQ,” “GAGQQ,” “GQGPY,” “AGQQ” or “SQ,” wherein some positions may be absent
  MISC_FEATURE (1397)..(1401): This region may encompass “SGGQQ,” “GAGQQ,” “GQGPY,” “AGQQ” or “SQ,” wherein some positions may be absent
  MISC_FEATURE (1405)..(1409): This region may encompass “SGGQQ,” “GAGQQ,” “GQGPY,” “AGQQ” or “SQ,” wherein some positions may be absent
  MISC_FEATURE (1413)..(1417): This region may encompass “SGGQQ,” “GAGQQ,” “GQGPY,” “AGQQ” or “SQ,” wherein some positions may be absent
  MISC_FEATURE (1354)..(1417): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (1421)..(1440): This region may encompass 6-20 residues
  MISC_FEATURE (1447)..(1451): This region may encompass “SGGQQ,” “GAGQQ,” “GQGPY,” “AGQQ” or “SQ,” wherein some positions may be absent
  MISC_FEATURE (1455)..(1459): This region may encompass “SGGQQ,” “GAGQQ,” “GQGPY,” “AGQQ” or “SQ,” wherein some positions may be absent
  MISC_FEATURE (1463)..(1467): This region may encompass “SGGQQ,” “GAGQQ,” “GQGPY,” “AGQQ” or “SQ,” wherein some positions may be absent
  MISC_FEATURE (1471)..(1475): This region may encompass “SGGQQ,” “GAGQQ,” “GQGPY,” “AGQQ” or “SQ,” wherein some positions may be absent
  MISC_FEATURE (1479)..(1483): This region may encompass “SGGQQ,” “GAGQQ,” “GQGPY,” “AGQQ” or “SQ,” wherein some positions may be absent
  MISC_FEATURE (1487)..(1491): This region may encompass “SGGQQ,” “GAGQQ,” “GQGPY,” “AGQQ” or “SQ,” wherein some positions may be absent
  MISC_FEATURE (1495)..(1499): This region may encompass “SGGQQ,” “GAGQQ,” “GQGPY,” “AGQQ” or “SQ,” wherein some positions may be absent
  MISC_FEATURE (1503)..(1507): This region may encompass “SGGQQ,” “GAGQQ,” “GQGPY,” “AGQQ” or “SQ,” wherein some positions may be absent
  MISC_FEATURE (1444)..(1507): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (1511)..(1530): This region may encompass 6-20 residues
  MISC_FEATURE (1537)..(1541): This region may encompass “SGGQQ,” “GAGQQ,” “GQGPY,” “AGQQ” or “SQ,” wherein some positions may be absent
  MISC_FEATURE (1545)..(1549): This region may encompass “SGGQQ,” “GAGQQ,” “GQGPY,” “AGQQ” or “SQ,” wherein some positions may be absent
  MISC_FEATURE (1553)..(1557): This region may encompass “SGGQQ,” “GAGQQ,” “GQGPY,” “AGQQ” or “SQ,” wherein some positions may be absent
  MISC_FEATURE (1561)..(1565): This region may encompass “SGGQQ,” “GAGQQ,” “GQGPY,” “AGQQ” or “SQ,” wherein some positions may be absent
  MISC_FEATURE (1569)..(1573): This region may encompass “SGGQQ,” “GAGQQ,” “GQGPY,” “AGQQ” or “SQ,” wherein some positions may be absent
  MISC_FEATURE (1577)..(1581): This region may encompass “SGGQQ,” “GAGQQ,” “GQGPY,” “AGQQ” or “SQ,” wherein some positions may be absent
  MISC_FEATURE (1585)..(1589): This region may encompass “SGGQQ,” “GAGQQ,” “GQGPY,” “AGQQ” or “SQ,” wherein some positions may be absent
  MISC_FEATURE (1593)..(1597): This region may encompass “SGGQQ,” “GAGQQ,” “GQGPY,” “AGQQ” or “SQ,” wherein some positions may be absent
  MISC_FEATURE (1534)..(1597): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (1601)..(1620): This region may encompass 6-20 residues
  MISC_FEATURE (1627)..(1631): This region may encompass “SGGQQ,” “GAGQQ,” “GQGPY,” “AGQQ” or “SQ,” wherein some positions may be absent
  MISC_FEATURE (1635)..(1639): This region may encompass “SGGQQ,” “GAGQQ,” “GQGPY,” “AGQQ” or “SQ,” wherein some positions may be absent
  MISC_FEATURE (1643)..(1647): This region may encompass “SGGQQ,” “GAGQQ,” “GQGPY,” “AGQQ” or “SQ,” wherein some positions may be absent
  MISC_FEATURE (1651)..(1655): This region may encompass “SGGQQ,” “GAGQQ,” “GQGPY,” “AGQQ” or “SQ,” wherein some positions may be absent
  MISC_FEATURE (1659)..(1663): This region may encompass “SGGQQ,” “GAGQQ,” “GQGPY,” “AGQQ” or “SQ,” wherein some positions may be absent
  MISC_FEATURE (1667)..(1671): This region may encompass “SGGQQ,” “GAGQQ,” “GQGPY,” “AGQQ” or “SQ,” wherein some positions may be absent
  MISC_FEATURE (1675)..(1679): This region may encompass “SGGQQ,” “GAGQQ,” “GQGPY,” “AGQQ” or “SQ,” wherein some positions may be absent
  MISC_FEATURE (1683)..(1687): This region may encompass “SGGQQ,” “GAGQQ,” “GQGPY,” “AGQQ” or “SQ,” wherein some positions may be absent
  MISC_FEATURE (1624)..(1687): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (1691)..(1710): This region may encompass 6-20 residues
  MISC_FEATURE (1717)..(1721): This region may encompass “SGGQQ,” “GAGQQ,” “GQGPY,” “AGQQ” or “SQ,” wherein some positions may be absent
  MISC_FEATURE (1725)..(1729): This region may encompass “SGGQQ,” “GAGQQ,” “GQGPY,” “AGQQ” or “SQ,” wherein some positions may be absent
  MISC_FEATURE (1733)..(1737): This region may encompass “SGGQQ,” “GAGQQ,” “GQGPY,” “AGQQ” or “SQ,” wherein some positions may be absent
  MISC_FEATURE (1741)..(1745): This region may encompass “SGGQQ,” “GAGQQ,” “GQGPY,” “AGQQ” or “SQ,” wherein some positions may be absent
  MISC_FEATURE (1749)..(1753): This region may encompass “SGGQQ,” “GAGQQ,” “GQGPY,” “AGQQ” or “SQ,” wherein some positions may be absent
  MISC_FEATURE (1757)..(1761): This region may encompass “SGGQQ,” “GAGQQ,” “GQGPY,” “AGQQ” or “SQ,” wherein some positions may be absent
  MISC_FEATURE (1765)..(1769): This region may encompass “SGGQQ,” “GAGQQ,” “GQGPY,” “AGQQ” or “SQ,” wherein some positions may be absent
  MISC_FEATURE (1773)..(1777): This region may encompass “SGGQQ,” “GAGQQ,” “GQGPY,” “AGQQ” or “SQ,” wherein some positions may be absent
  MISC_FEATURE (1714)..(1777): This region may encompass 4-8 repeating “GPG-X1” repeating units, wherein X1 is “SGGQQ,” “GAGQQ,” “GQGPY,” “AGQQ” or “SQ,” and some positions may be absent
  MISC_FEATURE (1781)..(1800): This region may encompass 6-20 residues
  MISC_FEATURE (1)..(1800): This sequence may encompass 2-20 “GGY-[GPG-X1]n1-GPS-(A)n2” repeating units, wherein X1 is “SGGQQ,” “GAGQQ,” “GQGPY,” “AGQQ” or “SQ,” n1 is 4-8 and n2 is 6-20 and some positions may be absent
GGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAAAAAAAAAAA

SEQ ID NO: 100 - protein, 5 aa, Artificial Sequence
  Description of Artificial Sequence Synthetic peptide
SGGQQ

SEQ ID NO: 101 - protein, 5 aa, Artificial Sequence
  Description of Artificial Sequence Synthetic peptide
GAGQQ

SEQ ID NO: 102 - protein, 5 aa, Artificial Sequence
  Description of Artificial Sequence Synthetic peptide
GQGPY

SEQ ID NO: 103 - protein, 4 aa, Artificial Sequence
  Description of Artificial Sequence Synthetic peptide
AGQQ

SEQ ID NO: 104 - protein, 70 aa, Artificial Sequence
  Description of Artificial Sequence Synthetic polypeptide
  MISC_FEATURE (7)..(11): This region may encompass “SGGQQ,” “GAGQQ,” “GQGPY,” “AGQQ” or “SQ,” wherein some positions may be absent
  MISC_FEATURE (15)..(19): This region may encompass “SGGQQ,” “GAGQQ,” “GQGPY,” “AGQQ” or “SQ,” wherein some positions may be absent
  MISC_FEATURE (23)..(27): This region may encompass “SGGQQ,” “GAGQQ,” “GQGPY,” “AGQQ” or “SQ,” wherein some positions may be absent
  MISC_FEATURE (31)..(35): This region may encompass “SGGQQ,” “GAGQQ,” “GQGPY,” “AGQQ” or “SQ,” wherein some positions may be absent
  MISC_FEATURE (39)..(43): This region may encompass “SGGQQ,” “GAGQQ,” “GQGPY,” “AGQQ” or “SQ,” wherein some positions may be absent
  MISC_FEATURE (47)..(51): This region may encompass “SGGQQ,” “GAGQQ,” “GQGPY,” “AGQQ” or “SQ,” wherein some positions may be absent
  MISC_FEATURE (55)..(59): This region may encompass “SGGQQ,” “GAGQQ,” “GQGPY,” “AGQQ” or “SQ,” wherein some positions may be absent
  MISC_FEATURE (63)..(67): This region may encompass “SGGQQ,” “GAGQQ,” “GQGPY,” “AGQQ” or “SQ,” wherein some positions may be absent
  MISC_FEATURE (4)..(67): This region may encompass 4-8 “GPG-X1” repeating units, wherein X1 is “SGGQQ,” “GAGQQ,” “GQGPY,” “AGQQ” or “SQ,” and some positions may be absent
GGYGPGXXXXXGPGXXXXXGPGXXXXXGPGXXXXXGPGXXXXXGPGXXXXXGPGXXXXXGPGXXXXXGPS

SEQ ID NO: 105 - protein, 20 aa, Artificial Sequence
  Description of Artificial Sequence Synthetic peptide
  MISC_FEATURE (1)..(20): This sequence may encompass 6-20 residues
AAAAAAAAAAAAAAAAAAAA

SEQ ID NO: 106 - protein, 5 aa, Artificial Sequence
  Description of Artificial Sequence Synthetic peptide
  MOD_RES (5)..(5): Any amino acid
GPGGX

SEQ ID NO: 107 - protein, 5 aa, Artificial Sequence
  Description of Artificial Sequence Synthetic peptide
GPGQQ

SEQ ID NO: 108 - protein, 90 aa, Artificial Sequence
  Description of Artificial Sequence Synthetic polypeptide
  MISC_FEATURE (7)..(11): This region may encompass “SGGQQ,” “GAGQQ,” “GQGPY,” “AGQQ” or “SQ,” wherein some positions may be absent
  MISC_FEATURE (15)..(19): This region may encompass “SGGQQ,” “GAGQQ,” “GQGPY,” “AGQQ” or “SQ,” wherein some positions may be absent
  MISC_FEATURE (23)..(27): This region may encompass “SGGQQ,” “GAGQQ,” “GQGPY,” “AGQQ” or “SQ,” wherein some positions may be absent
  MISC_FEATURE (31)..(35): This region may encompass “SGGQQ,” “GAGQQ,” “GQGPY,” “AGQQ” or “SQ,” wherein some positions may be absent
  MISC_FEATURE (39)..(43): This region may encompass “SGGQQ,” “GAGQQ,” “GQGPY,” “AGQQ” or “SQ,” wherein some positions may be absent
  MISC_FEATURE (47)..(51): This region may encompass “SGGQQ,” “GAGQQ,” “GQGPY,” “AGQQ” or “SQ,” wherein some positions may be absent
  MISC_FEATURE (55)..(59): This region may encompass “SGGQQ,” “GAGQQ,” “GQGPY,” “AGQQ” or “SQ,” wherein some positions may be absent
  MISC_FEATURE (63)..(67): This region may encompass “SGGQQ,” “GAGQQ,” “GQGPY,” “AGQQ” or “SQ,” wherein some positions may be absent
  MISC_FEATURE (4)..(67): This region may encompass 4-8 “GPG-X1” repeating units, wherein X1 is “SGGQQ,” “GAGQQ,” “GQGPY,” “AGQQ” or “SQ,” and some positions may be absent
  MISC_FEATURE (71)..(90): This region may encompass 6-20 residues
GGYGPGXXXXXGPGXXXXXGPGXXXXXGPGXXXXXGPGXXXXXGPGXXXXXGPGXXXXXGPGXXXXXGPSAAAAAAAAAAAAAAAAAAAA

SEQ ID NO: 109 - protein, 20 aa, Artificial Sequence
  Description of Artificial Sequence Synthetic peptide
  MISC_FEATURE (1)..(20): This sequence may encompass 4-20 residues
AAAAAAAAAAAAAAAAAAAA

SEQ ID NO: 110 - protein, 9 aa, Artificial Sequence
  Description of Artificial Sequence Synthetic peptide
  MISC_FEATURE (1)..(9): This sequence may encompass 7-9 residues
AAAAAAAAA

SEQ ID NO: 111 - protein, 64 aa, Artificial Sequence
  Description of Artificial Sequence Synthetic polypeptide
  MISC_FEATURE (4)..(8): This region may encompass “SGGQQ,” “GAGQQ,” “GQGPY,” “AGQQ” or “SQ,” wherein some positions may be absent
  MISC_FEATURE (12)..(16): This region may encompass “SGGQQ,” “GAGQQ,” “GQGPY,” “AGQQ” or “SQ,” wherein some positions may be absent
  MISC_FEATURE (20)..(24): This region may encompass “SGGQQ,” “GAGQQ,” “GQGPY,” “AGQQ” or “SQ,” wherein some positions may be absent
  MISC_FEATURE (28)..(32): This region may encompass “SGGQQ,” “GAGQQ,” “GQGPY,” “AGQQ” or “SQ,” wherein some positions may be absent
  MISC_FEATURE (36)..(40): This region may encompass “SGGQQ,” “GAGQQ,” “GQGPY,” “AGQQ” or “SQ,” wherein some positions may be absent
  MISC_FEATURE (44)..(48): This region may encompass “SGGQQ,” “GAGQQ,” “GQGPY,” “AGQQ” or “SQ,” wherein some positions may be absent
  MISC_FEATURE (52)..(56): This region may encompass “SGGQQ,” “GAGQQ,” “GQGPY,” “AGQQ” or “SQ,” wherein some positions may be absent
  MISC_FEATURE (60)..(64): This region may encompass “SGGQQ,” “GAGQQ,” “GQGPY,” “AGQQ” or “SQ,” wherein some positions may be absent
  MISC_FEATURE (1)..(64): This region may encompass 4-8 “GPG-X1” repeating units, wherein X1 is “SGGQQ,” “GAGQQ,” “GQGPY,” “AGQQ” or “SQ,” and some positions may be absent
GPGXXXXXGPGXXXXXGPGXXXXXGPGXXXXXGPGXXXXXGPGXXXXXGPGXXXXXGPGXXXXX

SEQ ID NO: 112 - protein, 23 aa, Artificial Sequence
  Description of Artificial Sequence Synthetic peptide
  MISC_FEATURE (4)..(23): This region may encompass 6-20 residues
GPSAAAAAAAAAAAAAAAAAAAA

(19) United States
(12) Patent Application Publication
(10) Pub. No.: US 2020/0399328 A1
(43) Pub. Date: Dec. 24, 2020

(54) SILK FIBERS

(71) Applicant: Bolt Threads, Inc., Emeryville, CA (US)

(72) Inventors: David BRESLAUER, San Francisco, CA (US); Lindsay WRAY, Benicia, CA (US); Joshua KITTLESON, Pleasant Hill, CA (US)

(21) Appl. No.: 16/921,799

(22) Filed: Jul. 6, 2020

Related U.S. Application Data
(63) Continuation of application No. 15/558,548, filed on Sep. 14, 2017, now abandoned.
(60) Provisional application No. 62/133,895, filed on Mar. 16, 2015.

(51) Int. Cl. C07K 14/435; D01D 5/24; D01D 5/253; D01F 6/68; C12P 21/02; D01D 5/06; D01F 4/02

(52) U.S. Cl. CPC C07K 14/43518; D01D 5/24; D01D 5/253; D01F 4/02; C12P 21/02; D01D 5/06; D01F 6/68

(57) ABSTRACT

Methods and compositions are provided for improved proteinaceous block copolymer fibers based on long repeat units having molecular weight of greater than about 10 kDal. Each repeat unit includes more than about 150 amino acid residues that are organized into a number of “quasi-repeat units.” The fibers have improved mechanical properties that better recapitulate those of the native silk fibers.

### RELATED APPLICATION DATA

This application is a 3-7, 9-11, 15, 16 and 18-21 continuation of U.S. application Ser. No. 15/558,548, filed Sep. 14, 2017. which is the National Stage of International Application No. PCT/US2016/022707, filed Mar. 16, 2016, which claims the benefit of U.S. Provisional Application No. 62/133,895, filed Mar. 16, 2015, the entire disclosure of each of which is incorporated by reference for all purposes.

### SEQUENCE LISTING

The instant application contains a Sequence Listing which has been submitted electronically in ASCII format and is hereby incorporated by reference in its entirety. Said ASCII copy, created on Jul. 6, 2020, is named BTT-003C1_SequenceListing.txt and is 245,332 bytes in size

### TECHNICAL FIELD

The present disclosure relates generally to silk fibers produced from spider silk proteins. Specifically, the present disclosure relates to improved spider silk proteins.

### BACKGROUND

Polymeric fibers synthesized from the polypeptides in spider silks are not commercially available due to the difficulty in commercial scale fabrication and the technical challenges in producing fibers that are manufacturable into threads, yarns, or other fibers.

Natural spider silk proteins are large (>150 kDa, >1000 amino acids) polypeptides divisible into three domains: an N-terminal non-repetitive domain (NTD), a repeat domain (REP), and a C-terminal non-repetitive domain (CTD). The repeat domain comprises approximately 90% of the natural polypeptide, while the NTD and CTD are relatively small (˜150, ˜100 amino acids respectively). The NTD and CTD are well-studied and are believed to confer to the entire polypeptide chain aqueous stability, pH sensitivity, and molecular alignment upon aggregation.

A single species of spider creates a variety of fibers, each of which is utilized for different functions. Examples of these different functions include draglines, web capture spirals, prey immobilization, and silks to protect an egg sac. Dragline silks have exceptional mechanical properties. They are very strong for their weight and diameters, and also exhibit a combination of high extensibility in conjunction with high ultimate tensile strength.

Amino acid composition and protein structure vary considerably between types of silks and species of spiders. For example, orb weaving spiders have six unique types of glands that produce different silk polypeptide sequences that are polymerized into fibers tailored to fit an environmental or lifecycle niche. The fibers are named for the gland they originate from and the polypeptides are labeled with the gland abbreviation, for example “Sp” for spidroin (short for spider fibroin). In orb weaver spiders, examples include Major Ampullate (MaSp, also called dragline), Minor Ampullate (MiSp), Flagelliform (Flag), Aciniform (AcSp), Tubuliform (TuSp), and Pyriform (PySp).

There is a common class of orb weaver MaSp dragline silks (e.g. Nephila clavipes MaSp1) where the repeat domains contain glycine-rich regions, which are associated with amorphous regions of the fiber (possibly containing alpha-helices and/or beta-turns), and poly-alanine regions, which are associated with the beta-sheet crystalline regions of the fiber. The amino acid composition and sequence, as well as the fiber formation details both affect the mechanical properties of the fiber.

While it is thought that commercial applications of spider silk are possible, spider silk cannot be commercially farmed and harvested in the same way that silkworm silk is. This is due, in part, to the aggressive and territorial nature of spiders. Therefore, synthetically produced spider silk is thought to be the most likely cost-effective and viable path to commercialization.

Currently, recombinant silk fibers are not commercially available and, with a handful of exceptions, are not produced in microorganisms outside of Escherichia coil and other gram-negative prokaryotes. Recombinant silks produced to date have largely consisted either of polymerized short silk sequence motifs or fragments of native repeat domains, sometimes in combination with NTDs and/or CTDs. While these methods are able to produce small scales of recombinant silk polypeptides (milligrams at lab scale, kilograms at bioprocessing scale) using intracellular expression and purification by chromatography or bulk precipitation, they have not scaled to match conventional textile fibers. Additional production hosts that have been utilized to make silk polypeptides include transgenic goats, transgenic silkworms, and plants. Similarly, these hosts have yet to enable commercial scale production of silk, presumably due to slow engineering cycles.

What is needed, therefore, are improved spider-silk derived recombinant protein designs, expression constructs for their production at high rates, microorganisms expressing these proteins, and synthetic fibers made from these proteins that exhibit many of the desirable mechanical and morphological properties of natural spider silk fibers.

### SUMMARY

In some embodiments the invention provides a proteinaceous block copolymer fiber, wherein the block copolymer comprises: at least two occurrences of a repeat unit, the repeat unit comprising: more than 150 amino acid residues and having a molecular weight of at least 10 kDal; an alanine-rich region with 6 or more consecutive amino acids, comprising an alanine content of at least 80%; a glycine-rich region with 12 or more consecutive amino acids, comprising a glycine content of at least 40% and an alanine content of less than 30%; and wherein the fiber comprises at least one property selected from the group consisting of a modulus of elasticity greater than 550 cN/tex, an extensibility of at least 10% and an ultimate tensile strength of at least 15 cN/tex.

In some embodiments, the repeat unit comprises from 150 to 1000 amino acid residues. In some embodiments, the repeat unit has a molecular weight from 10 kDal to 100 kDal.

In some embodiments, the repeat comprises from 2 to 20 alanine-rich regions.

In some embodiments, each alanine-rich region comprises from 6 to 20 consecutive amino acids, comprising an alanine content from 80% to 100%.

In some embodiments, the repeat comprises from 2 to 20 glycine-rich regions.

In some embodiments, each glycine-rich region comprises from 12 to 150 consecutive amino acids, comprising a glycine content from 40% to 80%.

In some embodiments, the modulus of elasticity is from 550 cN/tex to 1000 cN/tex.

In some embodiments, the extensibility is from 10% to 20%.

In some embodiments, the ultimate tensile strength is from 15 cN/tex to 100 cN/tex.

In some embodiments, the modulus of elasticity is greater than 550 cN/tex.

In some embodiments, the extensibility is at least 10%.

In some embodiments, the ultimate tensile strength is at least 15 cN/tex.

In some embodiments, the modulus of elasticity is greater than 550 cN/tex, the extensibility is at least 10%, and ultimate tensile strength is at least 15 cN/tex.

In some embodiments, each repeat unit has at least 95% sequence identity to a sequence that comprises from 2 to 20 quasi-repeat units, each quasi-repeat unit having a composition comprising {GGY-[GPG-X1]n1-GPS-(A)n2} (SEQ ID NO: 99), wherein for each quasi-repeat unit: X1 is independently selected from the group consisting of SGGQQ (SEQ ID NO: 100), GAGQQ (SEQ ID NO: 101), GQGPY (SEQ ID NO: 102), AGQQ (SEQ ID NO: 103), and SQ; and n1 is from 4 to 8, and n2 is from 6 to 20.

In some embodiments, a quasi repeat unit has at least 95% sequence identity to a MaSp2 dragline silk protein sequence.

In some embodiments, the invention provides for methods of synthesizing a proteinaceous block copolymer fiber by expressing a block copolymer of the present invention, formulating a spin dope comprising the expressed polypeptide and at least one solvent; and extruding the spin dope through a spinneret and through at least one coagulation bath to form the fiber, wherein the fiber comprises a property selected from the group consisting of a modulus of elasticity greater than 400 cN/tex, an extensibility of at least 10% and an ultimate tensile strength of at least 15 cN/tex.

In some embodiments, extruding the fiber through at least one coagulation bath comprises extruding the fiber sequentially through a first coagulation bath and a second bath, the first coagulation bath having a first chemical composition and the second bath having a second chemical composition different from the first chemical composition.

In some embodiments, the first chemical composition comprises a first solvent and at least one of a first acid and a first salt; and the second chemical composition comprises a second solvent and at least one of a second acid and a second salt; wherein the concentration of the second solvent is higher than the concentration of the first solvent, and wherein the first and second solvents are the same or different, and the first and second acids are the same or different.

In some embodiments, the fiber is translucent in the first coagulation bath.

### BRIEF DESCRIPTION OF THE DRAWINGS

FIG. 1 schematically illustrates a molecular structure of a block copolymer of the present disclosure, in an embodiment. FIG. 1 discloses SEQ ID NOS 111, 112 and 99, respectively, in order of appearance.

FIG. 2 is a magnified image of a fiber of the present disclosure having hollow core, in an embodiment.

FIG. 3 is a magnified image of a fiber of the present disclosure having a corrugated surface, in an embodiment.

FIGS. 4A-4D show mechanical properties measured from a plurality of fibers of the present disclosure, in embodiments.

FIG. 5 is a first stress-strain curve measured from a fiber of the present disclosure, in an embodiment.

FIG. 6 is a second stress-strain curve measured from a fiber of the present disclosure, in an embodiment.

FIG. 7 is a set of stress-strain curves measured from a fiber of the present disclosure, in an embodiment.

The figures depict various embodiments of the present disclosure for purposes of illustration only. One skilled in the art will readily recognize from the following discussion that alternative embodiments of the structures and methods illustrated herein may be employed without departing from the principles described herein.

### DETAILED DESCRIPTION

### Overview

Embodiments of the present disclosure include fibers synthesized from proteinaceous copolymers of recombinant spider silk proteins derived from MaSp2, such as from the species Argiope bruennichi. Each synthesized fiber contains protein molecules that include two to twenty repeat units, in which a molecular weight of each repeat unit is greater than about 20 kDal. Within each repeat unit of the copolymer are more than about 60 amino acid residues that are organized into a number of “quasi-repeat units.” In some embodiments, the repeat unit of a polypeptide described in this disclosure has at least 95% sequence identity to a MaSp2 dragline silk protein sequence.

Utilizing long polypeptides with fewer long exact repeat units has many advantages over utilizing polypeptides with a greater number of shorter exact repeat units to create a recombinant spider silk fiber. An important distinction is that a “long exact repeat” is defined as an amino acid sequence without shorter exact repeats concatenated within it. Long polypeptides with long exact repeats are more easily processed than long polypeptides with a greater number of short repeats because they suffer less from homologous recombination causing DNA fragmentation, they provide more control over the composition of amorphous versus crystalline domains, as well as the average size and size distribution of the nano-crystalline domains, and they do not suffer from unwanted crystallization during intermediate processing steps prior to fiber formation. Throughout this disclosure the term “repeat unit” refers to a subsequence that is exactly repeated within a larger sequence.

Throughout this disclosure, wherever a range of values is recited, that range includes every value falling within the range, as if written out explicitly, and further includes the values bounding the range. Thus, a range of “from X to Y” includes every value falling between X and Y, and includes X and Y.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared. Within this disclosure, a “region” is considered to be 6 or more amino acids in a continuous stretch within a polypeptide.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch. J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra),

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information. Such software also can be used to determine the mole percentage of any specified amino acid found within a polypeptide sequence or within a domain of such a sequence. As the person of ordinary skill will recognize such percentages also can be determined through inspection and manual calculation.

In embodiments, the morphology of the synthesized fibers includes fibers having a hollow cross-section or a corrugated outer surface with corrugations parallel to a longitudinal axis of a fiber. In embodiments, the synthesized fibers exhibit a strain to fracture of greater than 10%, or greater than 20%, or greater than 100%, or greater than 200%, or greater than 300%, or greater than 400%. In embodiments, the synthesized fibers exhibit a strain to fracture of from 1% to 400%, or from 1 to 200%, or from 1 to 100%, or from 1% to 20%, or from 10 to 200%, or from 10 to 100%, or from 10 to 50%, or from 10 to 20%, or from 50% to 150%, or from 100% to 150%, or from 300% to 400%. In embodiments, the synthesized fibers exhibit an elastic modulus greater 1500 MPa, or greater than 2000 MPa, or greater than 3000 MPa, or greater than 5000 MPa, or greater than 6000 MPa, or greater than 7000 MPa. In embodiments, the synthesized fibers exhibit an elastic modulus from 5200 to 7000 MPa, or from 1500 to 10000 MPa, or from 1500 to 8000 MPa, or from 2000 to 8000 MPa, or from 3000 to 8000 MPa, or from 5000 to 8000 MPa, or from 5000 to 6000 MPa, or from 6000 to 8000 MPa. In embodiments, the synthesized fibers exhibit an elastic modulus greater than 100 cN/tex, or greater than 200 cN/tex, or greater than 300 cN/tex, or greater than 400 cN/tex, or greater than 500 cN/tex, or greater than 550 cN/tex, or greater than 600 cN/tex. In embodiments, the synthesized fibers exhibit an elastic modulus from 100 to 600 cN/tex, or from 200 to 600 cN/tex, or from 300 to 600 cN/tex, or from 400 to 600 cN/tex, or from 500 to 600 cN/tex, or from 550 to 600 cN/tex, or from 550 to 575 cN/tex, or from 500 to 750 cN/tex, or from 500 to 1000 cN/tex, or from 500 to 1500 cN/tex. In embodiments, the synthesized fibers exhibit a maximum tensile strength greater than 100 MPa, or greater than 120 MPa, or greater than 140 MPa, or greater than 160 MPa, or greater than 180 MPa, or greater than 200 MPa, or greater than 220 MPa, or greater than 240 MPa, or greater than 260 MPa, or greater than 280 MPa, or greater than 300 MPa, or greater than 400 MPa, or greater than 600 MPa, or greater than 1000 MPa. In embodiments, the synthesized fibers exhibit a maximum tensile strength from 100 to 1000 MPa, or from 100 to 500 MPa, or from 100 to 300 MPa, or from 100 to 250 MPa, or from 100 to 200 MPa, or from 100 to 150 MPa. In embodiments, the synthesized fibers exhibit an ultimate tensile strength greater than 100 MPa, or greater than 120 MPa, or greater than 140 MPa, or greater than 160 MPa, or greater than 180 MPa, or greater than 200 MPa, or greater than 220 MPa, or greater than 240 MPa, or greater than 260 MPa, or greater than 260 MPa, or greater than 280 MPa, or greater than 300 MPa, or greater than 400 MPa, or greater than 600 MPa, or greater than 1000 MPa. In embodiments, the synthesized fibers exhibit an ultimate tensile strength from 100 to 1000 MPa, or from 100 to 500 MPa, or from 100 to 300 MPa, or from 100 to 250 MPa, or from 100 to 200 MPa, or from 100 to 150 MPa. In embodiments, the synthesized fibers exhibit a maximum tensile strength greater than 5 cN/tex, or greater than 10 cN/tex, or greater than 15 cN/tex, or greater than 20 cN/tex, or greater than 25 cN/tex. In embodiments, the synthesized fibers exhibit a maximum tensile strength from 5 to 30 cN/tex, or from 5 to 25 cN/tex, or from 10 to 30 cN/tex, or from 10 to 20 cN/tex, or from 15 to 20 cN/tex, or from 15 to 50 cN/tex, or from 15 to 75 cN/tex, or from 15 to 100 cN/tex. In embodiments, the synthesized fibers exhibit an ultimate tensile strength greater than 5 cN/tex, or greater than 10 cN/tex, or greater than 15 cN/tex, or greater than 20 cN/tex, or greater than 25 cN/tex. In embodiments, the synthesized fibers exhibit an ultimate tensile strength from 5 to 30 cN/tex, or from 5 to 25 cN/tex, or from 10 to 30 cN/tex, or from to 20 cN/tex, or from 15 to 20 cN/tex, or from 15 to 50 cN/tex, or from 15 to 75 cN/tex, or from 15 to 100 cN/tex. In some embodiments, the synthesized fibers exhibit a work of rupture greater than 0.2 cN*cm, or greater than 0.4 cN*cm, or greater than 0.8 cN*cm, or greater than 0.9 cN*cm, or greater than 1.3 cN*cm, or greater than 2 cN*cm, or from 0.2 to 2 cN*cm, or from 0.4 to 2 cN*cm, 0.6 to 2 cN*cm, or from 0.5 to 2 cN*cm, or from 0.5 to 1.3 cN*cm, or from 0.7 to 1.1 cN*cm. In some embodiments, the synthesized fibers exhibit linear density less than 5 dtex, or less than 3 dtex, or less than 2 dtex, or less than 1.5 dtex, or greater than 1.5 dtex, or greater than 1.7 dtex, or greater than 2 dtex, or from 1 to 5 dtex, or from 1 to 3 dtex, or from 1.5 to 2 dtex, or from 1.5 to 2.5 dtex.

### Molecular Structure

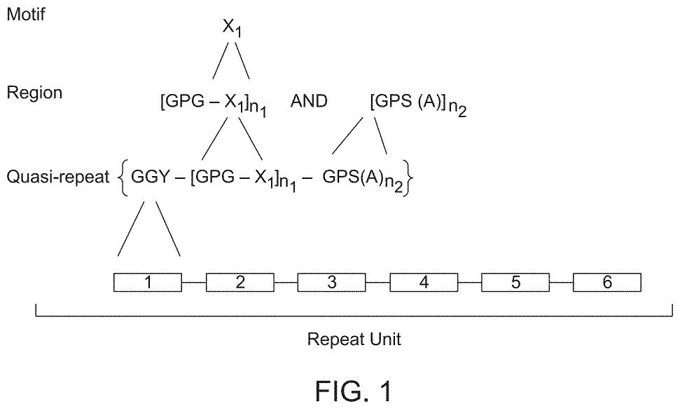

FIG. 1 schematically illustrates an example copolymer molecule of the present disclosure, in an embodiment. A block copolymer molecule of the present disclosure includes in each repeat unit more than 60, or more than 100, or more than 150, or more than 200, or more than 250, or more than 300, or more than 350, or more than 400, or more than 450, or more than 500, or more than 600, or more than 700, or more than 800, or more than 900, or more than 1000 amino acid residues, or from 60 to 1000, or from 100 to 1000, or from 200 to 1000, or from 300 to 1000, or from 400 to 1000, or from 500 to 1000, or from 150 to 1000, or from 150 to 400, or from 150 to 500, or from 150 to 750, or from 00 to 400, or from 200 to 500, or from 200 to 750, or from 250 to 350, or from 250 to 400, or from 250 to 500, or from 250 to 750, or from 250 to 1000, or from 300 to 500, or from 300 to 750 amino acid residues. Each repeat unit of the polypeptide molecules of this disclosure can have a molecular weight from 20 kDal to 100 kDal, or greater than 20 kDal, or greater than 10 kDal, or greater than 5 kDal, or from 5 to 60 kDal, or from 5 to 40 kDal, or from 5 to 20 kDal, or from 5 to 100 kDal, or from 5 to 50 kDal, or from 10 to 20 kDal, or from 10 to 40 kDal, or from 10 to 60 kDal, or from 10 to 100 kDal, or from 10 to 50 kDal, or from 20 to 100 kDal. or from 20 to 80 kDal, or from 20 to 60 kDal, or from 20 to 40 kDal, or from 20 to 30 kDal. A copolymer molecule of the present disclosure can include in each repeat unit more than 300 amino acid residues. A copolymer molecule of the present disclosure can include in each repeat unit about 315 amino acid residues. These amino acid residues are organized within the molecule at several different levels. A copolymer molecule of the present disclosure includes from 2 to 20 occurrences of a repeat unit. After concatenating the repeat unit, the polypeptide molecules of this disclosure can be from 20 kDal to 2000 kDal, or greater than 20 kDal, or greater than 10 kDal, or greater than 5 kDal, or from 5 to 400 kDal, or from 5 to 300 kDal, or from 5 to 200 kDal, or from 5 to 100 kDal, or from 5 to 50 kDal, or from 5 to 500 kDal, or from 5 to 1000 kDal, or from 5 to 2000 kDal, or from 10 to 400 kDal, or from 10 to 300 kDal, or from 10 to 200 kDal, or from 10 to 100 kDal, or from 10 to 50 kDal, or from 10 to 500 kDal, or from 10 to 1000 kDal, or from 10 to 2000 kDal, or from 20 to 400 kDal, or from 20 to 300 kDal, or from 20 to 200 kDal, or from 40 to 300 kDal, or from 40 to 500 kDal, or from 20 to 100 kDal, or from 20 to 50 kDal, or from 20 to 500 kDal, or from 20 to 1000 kDal, or from 20 to 2000 kDal. As shown in FIG. 1, each “repeat unit” of a copolymer fiber comprises from two to twenty “quasi-repeat” units (i.e., n3 is from 2 to 20). Quasi-repeats do not have to be exact repeats. Each repeat can be made up of concatenated quasi-repeats. Equation 1 shows the composition of a quasi-repeat unit according the present disclosure

{GGY-[GPG-X₁]ₙ₁-GPS-(A)ₙ₂}ₙ₃ (SEQ ID NO:99). (Equation 1)

The variable compositional element X₁ (termed a “motif”) is according to any one of the following amino acid sequences shown in Equation 2 and X₁ varies randomly within each quasi-repeat unit.

X₁=SGGQQ (SEQ ID NO: 100) or GAGQQ (SEQ ID NO: 101) or GQGPY (SEQ ID NO: 102) or AGQQ (SEQ ID NO: 103) or SQ (Equation 2)

Referring again to Equation 1. the compositional element of a quasi-repeat unit represented by “GGY-[GPG-X₁]n₁-GPS” (SEQ ID NO: 104) in Equation 1 is referred to a “first region.” A quasi-repeat unit is formed, in part by repeating from 4 to 8 times the first region within the quasi-repeat unit. That is, the value of n₁ indicates the number of first region units that are repeated within a single quasi-repeat unit, the value of n₁ being any one of 4, 5, 7 or 8. The compositional element represented by “(A)n₂” (SEQ ID NO: 105) is referred to a “second region” and is formed by repeating within each quasi-repeat unit the amino acid sequence “A” n₂ times (SEQ ID NO: 105). That is, the value of n₂ indicates the number of second region units that are repeated within a single quasi-repeat unit, the value of n₂ being any one of 6, 7, 8, 9, 10, 11, 12, 13, 14, 15, 16, 17, 18, 19, or 20. In some embodiments, the repeat unit of a polypeptide of this disclosure has at least 95% sequence identity to a sequence containing quasi-repeats described by Equations 1 and 2. In some embodiments, the repeat unit of a polypeptide of this disclosure has at least 80%, or at least 90%, or at least 95%, or at least 99% sequence identity to a sequence containing quasi-repeats described by Equations 1 and 2.

The first region described in Equation 1 is considered a glycine-rich region. A region can be glycine-rich if 6 or more consecutive amino acids within a sequence are more than 45% glycine. A region can be glycine-rich if 12 or more consecutive amino acids within a sequence are more than 45% glycine. A region can be glycine-rich if 18 or more consecutive amino acids within a sequence are more than 45% glycine. A region can be glycine-rich if 4 or more, or 6 or more, or 10 or more, or 12 or more, or 15 or more, or 20 or more, or 25 or more, or 30 or more, or 40 or more, or 50 or more, or 60 or more, or 70 or more, or 80 or more, or 100 or more, or 150 or more consecutive amino acids within a sequence are more than 30%, or more than 40%, or more than 45%, or snore than 50%, or more than 55% glycine, or more than 60% glycine, or more than 70% glycine, or more than 80% glycine, or from 30% to 80%, or from 40% to 80%, or from 45% to 80%, or from 30% to 55%, or from 30% to 50%, or from 30% to 45%, or from 30% to 40%, or from 40% to 50%, or 40% to 55%, or 40% to 60% glycine. A region can be glycine-rich if from 5 to 150, or from 10 to 150, or from 12 to 150, or from 12 to 100, or from 12 to 80, or from 12 to 60, or from 20 to 60 consecutive amino acids within a sequence are more than 30%, or more than 40%, or more than 45%, or more than 50%, or more than 55% glycine, or more than 60% glycine, or more than 70% glycine, or more than 80% glycine, or from 30% to 80%, or from 40% to 80%, or from 45% to 80%, or from 30% to 55%, or from 30% to 50%, or from 30% to 45%, or from 30% to 40%, or from 40% to 50%, or 40% to 55%, or 40% to 60% glycine. In addition, a glycine-rich region can have less than 10%, or less than 20%, or less than 30%, or less than 40% alanine, or from about 0% to 10%, or from about 0% to 20%, or from about 0% to 30%, or from about 0% to 40%, or alanine. A region can be alanine-rich if 4 or more, or 6 or more, or 8 or more, or 10 or more consecutive amino acids within a sequence are more than 70%, or more than 75%, or more than 80%, or more than 85%, or more than 90% alanine, or from 70% to about 100%, or from 75% to about 100%, or from 80% to about 100%, or from 85% to about 100%, or from 90% to about 100% alanine. A region can be alanine-rich if from 4 to 10, or from 4 to 12, or from 4 to 15, or from 6 to 10, or from 6 to 12, or from 6 to 15, or from 4 to 20, or from 6 to 20 consecutive amino acids within a sequence are more than 70%, or more than 75%, or more than 80%, or more than 85%, or more than 90% alanine, or from 70% to about 100%, or from 75% to about 100%, or from 80% to about 100%, or from 85% to about 100%, or from 90% to about 100% alanine. The repeats described in this disclosure can have 6, or more than 2, or more than 4 or more than 6, or more than 8, or more than 10, or more than 15, or more than 20, or from 2 to 25, or from 2 to 10, or from 4 to 10, or from 2 to 8, or from 4 to 8 alanine-rich regions. The repeats described in this disclosure can have 6, or more than 2, or more than 4 or more than 6, or more than 8, or more than 10, or more than 15, or more than 20, or from 2 to 25, or from 2 to 10, or from 4 to 10, or from 2 to 8, or from 4 to 8 glycine-rich regions.

As further described below, one example of a copolymer molecule includes three “long” quasi-repeats followed by three “short” quasi-repeat units. A “long” quasi-repeat unit is comprised of quasi-repeat units that do not use the same X₁ constituent (as shown in Equation 2) more than twice in a row, or more than two times in a repeat unit. Each “short” quasi-repeat unit includes any of the amino acid sequences identified in Equation 2, but regardless of the amino acid sequences used, the same sequences are in the same location within the molecule. Furthermore, in this example copolymer molecule, no more than 3 quasi-repeats out of 6 share the same X₁. “Short” quasi-repeat units are those in which n1=4 or 5 (as shown in Equation 1). Long quasi-repeat units are defined as those in which n1=6, 7 or 8 (as shown in Equation 1).

In some embodiments, the repeat unit of the copolymer is composed of X_{qr} quasi-repeat units, where X_{qr} is a number from 2 to 20, and the number of short quasi-repeat units is X_{sqr} and the number of long quasi-repeat units is X_{lqr}, where

X_{sqr}X_{lqr}=X_{qr} (Equation 3)

and X_{sqr} is a number from 1 to (X_{qr}−1) and X_{lqr} is a number from 1 to (X_{qr}−1).

In another embodiment, n1 is from 4 to 5 for at least half of the quasi-repeat units. In yet another embodiment, n2 is from 5 to 8 for at least half of the quasi-repeat units.

One feature of copolymer molecules of the present disclosure is the formation of nano-crystalline regions that, while not wishing to be bound by theory, are believed to form from the stacking of beta-sheet regions, and amorphous regions composed of alpha-helix structures, beta-turn structures, or both. Poly-alanine regions (or in some species (GA)ₙ regions) in a molecule form crystalline beta-sheets within major ampullate (MA) fibers. Other regions within a repeat unit of major ampullate and flagelliform spider silks (for example containing GPGGX (SEQ ID NO: 106), GAGQQ (SEQ ID NO: 107), GGX where X=A, S or Y, GPG, SGGQQ (SEQ ID NO: 100), GAGQQ (SEQ ID NO: 101), GQGPY (SEQ ID NO: 102), AGQQ (SEQ ID NO: 103), and SQ, may form amorphous rubber-like structures that include alpha-helices and beta-turn containing structures. Furthermore, secondary, tertiary and quaternary structure is imparted to the morphology of the fibers via amino acid sequence and length, as well as the conditions by which the fibers are formed, processed and post-processed. Materials characterization techniques (such as NMR, FTIR and x-ray diffraction) have suggested that the poly-alanine crystalline domains within natural MA spider silks and recombinant silk derived from MA spider silk sequences are typically very small (<10 nm). Fibers can be highly crystalline or highly amorphous, or a blend of both crystalline and amorphous regions, but fibers with optimal mechanical properties have been speculated to be composed of 10-40% crystalline material by volume. In some embodiments, the repeat unit of a polypeptide described in this disclosure has at least 80%, or at least 90%, or at least 95%, or at least 99% sequence identity to a MA dragline silk protein sequence. In some embodiments, the repeat unit of a polypeptide described in this disclosure has at least 80%, or at least 90%, or at least 95%, or at least 99% sequence identity to a MaSp2 dragline silk protein sequence. In some embodiments, the repeat unit of a polypeptide described in this disclosure has at least 80%, or at least 90%, or at least 95%, or at least 99% sequence identity to a spider dragline silk protein sequence. In some embodiments, a quasi repeat unit of a polypeptide described in this disclosure has at least 80%, or at least 90%, or at least 95%, or at least 99% sequence identity to a MA dragline silk protein sequence. In some embodiments, a quasi repeat unit of a polypeptide described in this disclosure has at least 80%, or at least 90%, or at least 95%, or at least 99% sequence identity to a MaSp2 dragline silk protein sequence. In some embodiments, a quasi repeat unit of a polypeptide described in this disclosure has at least 80%, or at least 90%, or at least 95%, or at least 99% sequence identity to a spider dragline silk protein sequence.

The repeat unit of the proteinaceous block copolymer that forms fibers with good mechanical properties can be synthesized using a portion of a silk polypeptide. Some exemplary sequences that can be used as repeats in the proteinaceous block copolymers of this disclosure are shown in Table 1. These polypeptide repeat units contain alanine-rich regions and glycine-rich regions, and are 150 amino acids in length or longer. These exemplary sequences were demonstrated to express using a Pichia expression system as taught in co-owned PCT Publication WO 2015042164.

TABLE 1
Exemplary sequences that can be used as repeat units

| Seq. ID | |
|---|---|
| No. | AA |
| 1 | GGYGPGAGQQGPGSGGQQGPGGQGPYGSGQQGPGGAGQQGPGGQGPYGPGAAAAAAA |
| | AAGGYGPGAGQQGPGGAGQQGPGSQGPGGQGPYGPGAGQQGPGSQGPGSGGQQGPGG |
| | QGPYGPSAAAAAAAAAGGYGPGAGQRSQGPGGQGPYGPGAGQQGPGSQGPGSGGQQG |
| | PGGQGPYGSAAAAAAAAGGYGPGAGQQGPGSQGPGSGGQQGPGGQGPYGPGAAAAA |
| | AAVGGYGPGAGQQGPGSQGPGSGGQQGPGGQGPYGPSAAAAAAAAGGYGPGAGQQGP |
| | GSQGPGSGGQQGPGGQGPYGPSAAAAAAAA |
| 2 | GGQGGRGGFGGLGSQGAGGAGQGGAGAAAAAAAAGGDGGSGLGGYGAGRGHGVGLGG |
| | AGGAGAASAAAAAGGQGGRGGFGGLGSQGAGGAGQGGAGAAAAAAAAGGDGGSGLGG |
| | YGAGRGHGAGLGGAGGAGAASAAAAAGGQGGRGGFGGLGSQGSGGAGQGGSGAAAAA |
| | AAAGGDGGSGLGGYGAGRGYGAGLGGAGGAGAASAAAAAGGQGGRGGFGGLGSQGAG |
| | GAGQGGSGAAAAAAAAVADGGSGLGGYGAGRGYGAGLGGAGGAGAASAAAAT |
| 3 | GSAPQGAGGPAPQGPSQQGPVSQGPYGPGAAAAAAAAGGYGPGAGQQGPGSQGPGSG |
| | GQQGPGSQGPGSGGQQGPGGQGPYGPSAAAAAAAAAGGYGPGAGGOGPGSQGPGSGG |
| | QQGPGGQGPYGPGAAAAAAAVGGYGPGAGQQGPGSQGPGSGGQQGPGGQGPYGPSAA |
| | AAAAAAGGYGPGAGQQGPGSQGPGSGGQQGPGGQGPYGPSAAAAAAAAGGYGPGAGQ |
| | QGPGSGGQQGPGGQGPYGSGQQGPGGAGQQGPGGQGPYGPGAAAAAAAAA |
| 4 | GGYGPGAGQQGPGSGGQQGPGGQGPYGSGQQGPGGAGQQGPGGQGPYGPGAAAAAAA |
| | AAGGYGPGAGQQGPGGAGQQGPGSQGPGGQGPYGPGAGQQGPGSQGPGSGGQQGPGG |
| | QGPYGPSAAAAAAAAGGYGPGAGQQGPGSQGPGSGGQQGPGGQGPYGPSAAAAAAAA |
| | GGYGPGAGQQGPGSGGQQGPGGQGPYGSGQQGPGGAGQQGPGGQGPYGGGYGPGAGQ |
| | QGPGSQGPGSGGQQGFGGQGPYGPSAAAAAAAA |
| 5 | GPGARRQGPGSQGPGSGGQQGPGGQGPYGSGQQGPGGAGQQGPGGQGPYGPGAAAAA |
| | AAAAGGYGPGAGQQGPGGAGQQGPGSQGPGGQGPYGPGAGQQGPGSQGPGSGGQQGP |
| | GGQGPYGPSAAAAAAAAAGGYGPGAGQQGPGSQGPGSGGQQGPGGQGPYGPGAAAAA |
| | AAVGGYGPGAGQOGPGSQGPGSGGQQGPGGQGPYGPSAAAAAAAAGGYGPGAGQQGP |
| | GSQGPGSGGQQGPGGQGPYGPSAAAAAAAA |
| 6 | GPGARRQGPGSQGPGSGGQQGPGGQGPYGSGQQGPGGAGQQGPGGQGPYGPGAAAAA |
| | AAAAGGYGPGAGQQGPGGAGQQGPGSQGPGGQGPYGPGAGQQGPGSQGPGSGGQQGP |
| | GGQGPYGPSAAAAAAAAGGYGPGAGQQGPGSQGPGSGGQQGPGGQGPYGPGAAAAAA |
| | AVGGYGPGAGQQGPGSQGPGSGGQQGPGGQGPYGPSAAAAAAAAGGYGPGAGQQGPG |
| | SQGPGSGGQQGPGGQGPYGPSAAAAAAAA |
| 7 | GGYGPGAGQQGPGSGGQQGPGGQGPYGSGQQGPGGAGQQGPGGQGPYGPGAAAAAAA |
| | AAGGYGPGAGQQGPGGAGQQGPEGPGSQGPGSGGQQGPGGQGPYGPGAAAAAAAVGG |
| | YGPGAGQQGPGSQGPGSGGQQGPGGQGPYGPSAAAAAAAAGGYGPGAGQQGPGSQGP |
| | GSGGQQGPGGQGPYGPSAAAAAAAAGGYGPGAGQQGPGSGGQQGPGGQGPYGSGQQG |
| | PGGAGQQGPGGQGPYGPGAAAAAAAAA |
| 8 | GVFSAGQGATPWENSQLAESFISRFLRFIGQSGAFSPNQLDDMSSIGDTLKTAIEKM |
| | AQSRKSSKSKLQALNMAFASSMAEIAVAEQGGLSLEAKTNAIASALSAAFLETTGYV |
| | NQQFVNEIKTLIFMIAQASSNEISGSAAAAGGSSGGGGGSGQGGYGQGAYASASAAA |
| | AYGSAPQGTGGPASQGPSQQGPVSQPSYGPSATVAVTAVGGRPQGPSAPRQQGPSQQ |
| | GPGQQGPGGRGPYGPSAAAAAAAA |
| 9 | GAGAGAGAGAGAGAGAGSGASTSVSTSSSSGSGAGAGAGSGAGSGAGAGSGAGAGAG |
| | AGGAGAGFGSGLGLGYGVGLSSAQAQAQAQAAAQAQAQAQAQAYAAAQAQAQAQAQA |
| | QAAAAAAAAAAAGAGAGAGAGAGAGAGAGSGASTSVSTSSSSGSGAGAGAGSGAGSG |
| | AGAGSGAGAGAGAGGAGAGFGSGLGLGYGVGLSSAQAQAQAQAAAQAQAQAQAQAYA |
| | AAQAQAQAQAQAQAAAAAAAAAAA |
| 10 | GAGAGAGAGAGAGAGAGSGASTSVSTSSSSGSGAGAGAGSGAGSGAGAGSGAGAGAG |
| | AGGAGAAFGSGLGLGYGVGLSSAQAQAQAQAAAQAQADAQAQAYAAAQAQAQAQAQA |
| | QAAAAAAAAAAAGAGAGAGAGSGAGAGAGSGASTSVSTSSSSGSGAGAGAGSGAGSG |
| | AGAGSGAGAGAGAGGAGAGFGSGLGLGYGVGLSSAQAQAQAQAAAQAQADAQAQAYA |
| | AAQAQAQAQAQAQAAAAAAAAAAA |
| 11 | GAGAGAGAGSGAGAGAGSGASTSVSTSSSSGSGAGAGAGSGAGSGAGAGSGAGAGAG |
| | AGGAGAGFGSGLGLGYGVGLSSAQAQAQSAAAARAQADAQAQAYAAAQAQAQAQAQA |
| | QAAAAAAAAAAAGAGAGAGAGAGAGAGAGSGASTSVSTSSSSASGAGAGAGSGAGSG |
| | AGAGSGAGAGAGAGGAGAGFGSGLGLGYGVGLSSAQAQAQAQAAAQAQAQAQAQALA |
| | AAQAQAQAQAQAQAAAATAAAAAA |
| 12 | GGYGPGAGQQGPGGAGQQGFGSQGPGGQGPYGPGAGQQGPGSQGPGSGGQQGPGGQG |
| | PYGPSAAAAAAAAGGYGPGAGQQGPGSQGPGSGGQQGPGSQGPGSGGQQGPGGQGPY |
| | GPSAAAAAAAAAGGYGPGAGQQGPGSQGPGSGGQQGPGGQGPYGPGAAAAAAAVGGY |
| | GPGAGQQGPGSQGPGSGGQQGPGGQGPYGPSAAAAAAAAGGYGPGAGQQGPGSQGPG |
| | SGGQQGPGGQGPYGPSAAAAAAAA |
| 13 | GGYGPGAGQQGPGGAGQQGPGSQGPGGQGPYGPGAGQQGPGSQGPGSGGQQGPGGQG |
| | PYGPSAAAAAAAAGGYGPGAGQQGPGSQGPGSGGQQGPGSQGPGSGGQQGPGGQGPY |
| | GPSAAAAAAAAGGYGPGAGQQGPGSQGPGSGGQQGPGGQGPYGPGAAAAAAAVGGYG |
| | PGAGQQGPGSQGPGSGGQQGPGGQGPYGPSAAAAAAAAGGYGPGAGQQGPGSQGPGS |
| | GGQQGPGGQGPYGPSAAAAAAAA |
| 14 | GHQGPHRKTPWETPEMAENFMNNVRENLEASRIFPDELMKDMEAITNTMIAAVDGLE |
| | AQHRSSYASLQAMNTAFASSMAQLFATEQDYVDTEVIAGAIGKAYQQITGYENPHLA |
| | SEVTRLIQLFREEDDLENEVEISFADTDNAIARAAAGAAAGSAAASSSADASATAEG |
| | ASGDSGFLFSTGTFGRGGAGAGAGAAAASAAAASAAAAGAEGDRGLFFSTGDFGRGG |
| | AGAGAGAAAASAAAASAAAA |
| 15 | GGAQKHPSGEYSVATASAAATSVTSGGAPVGKPGVPAPIFYPQGPLQQGPAPGPSNV |
| | QPGTSQQGPIGGVGESNTFSSSFASALGGNRGFSGVISSASATAVASAFQKGLAPYG |
| | TAFALSAASAAADAYNSIGSGASASAYAQAFARVLYPLLQQYGLSSSADASAFASAI |
| | ASSFSTGVAGQGPSVPYVGQQQPSIMVSAASASAAASAAAVGGGPVVQGPYDGGQPQ |
| | QPNIAASAAAAATATSS |
| 16 | GGQGGRGGFGGLGSQGEGGAGQGGAGAAAAAAAAGADGGFGLGGYGAGRGYGAGLGG |
| | AGGAGAASAAAAAGGQGGRSGFGGLGSQGAGGAGQGGAGAAAAAAAAGADGGSGLGG |
| | YGAGRGYGASLGGADGAGAASAAAAAGGQGGRGGFGGLGSQGAGGAGQGGAGAAAAA |
| | AAASGDGGSGLGGYGAGRGYGAGLGGAGGAGAASAAAAAGGEGGRGGFGGLGSQGAG |
| | GAGQGGSLAAAAAAAA |
| 17 | GPGGYGGPGQPGPGQGQYGPGPGQQGPRQGGQQGPASAAAAAAAGPGGYGGPGQQGP |
| | RQGQQQGPASAAAAAAAAAAGPRGYGGPGQQGPVQGGQQGPASAAAAAAAAGVGGYG |
| | GPGQQGPGQGQYGPGTGQQGQGPSGQQGPAGAAAAAAGGAAGPGGYGGPGQQGPGQG |
| | QYGPGTGQOGQGPSGQQGPAGAAAAAAAAAGPGGYGGPGQQGPGCGQYGPGAGQQGQ |
| | GPGSQQGPASAAAAAA |
| 18 | GSGAGQGTGAGAGAAAAAAGAAGSGAGQGAGSGAGAAAAAAAASAAGAGQGAGSGSG |
| | AGAAAAAAAAAGAGQGAGSGSGAGAAAAAAAAAAAAQQQQQQQAAAAAAAAAAAAAG |
| | SGQGASFGVTQQFGAPSGAASSAAAAAAAAAAAAAGSGAGQEAGTGAGAAAAAAAAG |
| | AAGSGAGQGAGSGAGAAAAAAAAASAAGAGQGAGSGSGAGAAAAAAAAAAAAQQQQQ |
| | QQAAAAAAAAAAAAA |
| 19 | GGAQKQPSGESSVATASAAATSVTSAGAPVGKPGVPAPIFYPQGPLQQGPAPGPSYV |
| | QPATSQQGPIGGAGRSNAFSSSFASALSGNRGFSEVISSASATAVASAFQKGLAPYG |
| | TAFALSAASAAADAYNSIGSGANAFAYAQAFARVLYPLVQQYGLSSSAKASAFASAI |
| | ASSFSSGAAGQGQSIPYGGQQQPPMTISAASASAGASAAAVKGGQVGQGPYGGQQQS |
| | TAASASAAATTATA |
| 20 | GADGGSGLGGYGAGRGYGAGLGGADGAGAASAAAAAGGQGGRGGFGRLGSQGAGGAG |
| | QGGAGAAAAVAAAGGDGGSGLGGYGAGRGYGAGLGGAGGAGAASAAAAAGGQGGRGG |
| | FGGLGSQGAGGAGQGGAGAAASGDGGSGLGGYGAGRGYGAGLGGADGAGAASAASAA |
| | GGQGGRGGFGGLGSQGAGGAGQGGAGAAAAAATAGGDGGSGLGGYGAGRGYGAGLGG |
| | AGGAGAASAAAAA |
| 21 | GAGAGQGGRGGYGQGGFGGQGSGAGAGASAAAGAGAGQGGRGGYGQGGFGGQGSGAG |
| | AGASAAAGAGAGQGGRGGYGQGGFGGQGSGAGAGASAAAAAGAGQGGRGGYGQGGLG |
| | GSGSGAGAGAGAAAAAAAGAGGYGQGGLGGYGQGAGAGQGGLGGYGSGAGAGASAAA |
| | AAGAGGAGQGGLGGYGQGAGAGQGGLGGYGSGAGAGAAAAAAAGAGGSGQGGLGGYG |
| | SGGGAGGASAAAA |
| 22 | GAYAYAYAIANAFASILANTGLLSVSSAASVASSVASAIATSVSSSSAAAAASASAA |
| | AAASAGASAASSASASSSASAAAGAGAGAGAGASGASGAAGGSGGFGLSSGFGAGIG |
| | GLGGYPSGALGGLGIPSGLLSSGLLSPAANQRIASLIPLILSAISPNGVNFGVIGSN |
| | IASLASQISQSGGGIAASQAFTQALLELVAAFIQVLSSAQIGAVSSSSASAGATANA |
| | FAQSLSSAFAG |
| 23 | GAAQKQPSGESSVATASAAATSVTSGGAPVGKPGVPAPIFYPQGPLQQGPAPGPSNV |
| | QPGTSQQGPIGGVGGSNAFSSSFASALSLNRGFTEVISSASATAVASAFQKGLAPYG |
| | TAFALSAASAAADAYNSIGSGANAFAYAQAFARVLYPLVRQYGLSSSGKASAFASAI |
| | ASSFSSGTSGQGPSIGQQQPPVTISAASASAGASAAAVGGGQVGQGPYGGQQQSTAA |
| | SASAAAATATS |
| 24 | GAAQKOPSGESSVATASAAATSVTSGGAPVGKPGVPAPIFYPQGPLQQGPAPGPSNV |
| | QPGTSQQGPIGGVGGSNAFSSSFASALSLNRGFTEVISSASATAVASAFQKGLAPYG |
| | TAFALSAASAAADAYNSIGSGANAFAYAQAFARVLYPLVRQYGLSSSGKASAFASAI |
| | ASSFSSGTSGOGPSIGQQQPPVTISAASASAGASAAAVGGGQVGQGPYGGQQQSTAA |
| | SASAAAATATS |
| 25 | GAAQKQPSGESSVATASAAATSVTSGGAPVGKPGVPAPIFYPQGPLQQGPAPGPSNV |
| | QPGTSQQGPIGGVGGSNAFSSSFASALSLNRGFTEVISSASATAVASAFQKGLAPYG |
| | TAFALSAASAAADAYNSIGSGANAFAYAQAFARVLYPLVQQYGLSSSAKASAFASAI |
| | ASSFSSGTSGQGPSIGQQQPPVTISAASASAGASAAAVGGGQVGQGPYGGQQQSTAA |
| | SASAAAATATS |
| 26 | GGAQKQPSGESSVATASAAATSVTSAGAPVGKPGVPAPIFYPQGPLQQGPAPGPSNV |
| | QPGTSQQGPIGGVGGSNAFSSSFASALSLNRGFTEVISSASATAVASAFQKGLAPYG |
| | TAFALSAASAAADAYNSIGSGANAFAYAQAFARVLYPLVQQYGLSSSAKASAFASAI |
| | ASSFSSGTSGQGPSNGQQQPPVTISAASASAGASAAAVGGGQVSQGPYGGQQQSTAA |
| | SASAAAATATS |
| 27 | GGAQKQPSGESSVATASAAATSVTSAGAPGGKPGVPAPIFYPQGPLQQGPAPGPSNV |
| | QPGTSQQGPIGGVGGSNAFSSSFASALSLNRGFTEVISSASATAVASAFQKGLAPYG |
| | TAFALSAASAAADAYNSIGSGANAFAYAQAFARVLYPLVQQYGLSSSAKASAFASAI |
| | ASSFSSGTSGQGPSIGQQQPPVTISAASASAGASAAAVGGGQVGQGPYGGQQQSTAA |
| | SASAAAATATS |
| 28 | GPGGYGGPGQQGPGQGQQQGPASAAAAAAAAGPGGYGGPGQQGPGQGQQQGPASAAA |
| | AAAAAAGPGGYGGPGQQRPGQAQYGRGTGQQGQGPGAQQGPASAAAAAAAGAGLYGG |
| | PGQQGPGQGQQQGPASAAAAAAAAAAAGPGGYGGPGQQGPGQAQQQGPASAAAAAAA |
| | GPGGYSGPGQQGPGQAQQQGPASAAAAAAAAAGPGGYGGPGQQGPGQGQQQGPASAA |
| | AAAAATAA |
| 29 | GAGGDGGLFLSSGDFGRGGAGAGAGAAAASAAAASSAAAGARGGSGFGVGTGGFGRG |
| | GAGDGASAAAASAAAASAAAAGAGGDSGLFLSSGDFGRGGAGAGAGAAAASAAAASA |
| | AAAGTGGVGGLFLSSGDFGRGGAGAGAGAAAASAAAASSAAAGARGGSGFGVGTGGF |
| | GRGGPGAGTGAAAASAAAASAAAAGAGGDSGLFLSSEDFGRGGAGAGTGAAAASAAA |
| | ASAAAA |
| 30 | GAGRGYGGGYGGGAAAGAGAGAGAGRGYGGGYGGGAGSGAGSGAGAGGGSGYGRGAG |
| | AGAGAGAAAAAGAGAGGAGGYGGGAGAGAGASAAAGAGAGAGGAGGYGGGYGGGAGA |
| | GAGAGAAAAAGAGAGAGAGRGYGGGFGGGAGSGAGAGAGAGGGSGYGRGAGGYGGGY |
| | GGGAGTGAGAAAATGAGAGAGAGRGYGGGYGGGAGAGAGAGAGAGGGSGYGRGAGAG |
| | ASVAA |
| 31 | GALGQGASVWSSPQMAENFMNGFSMALSQAGAFSGQEMKDFDDVRDIMNSAMDKMIR |
| | SGKSGRGAMRAMNAAFGSAIAEIVAANGGKEYQIGAVLDAVTNTLLQLTGNADNGFL |
| | NEISRLITLFSSVEANDVSASAGADASGSSGPVGGYSSGAGAAVGQGTAQAVGYGGG |
| | AQGVASSAAAGATNYAQGVSTGSTQNVATSTVTTTTNVAGSTATGYNTGYGIGAAAG |
| | AAA |
| 32 | GGQGGQGGYDGLGSQGAGQGGYGQGGAAAAAAAASGAGSAQRGGLGAGGAGOGYGAG |
| | SGGQGGAGQGGAAAATAAAAGGQGGQGGYGGLGSQGSGQGGYGQGGAAAAAAAASGD |
| | GGAGQEGLGAGGAGQGYGAGLGGQGGAGQGGAAAAAAAAAGGQGGQGGYGGLGSQGA |
| | GQGGYGQGGAAAAAAAASGAGGAGQGGLGAAGAGQGYGAGSGGQGGAGQGGAAAAAA |
| | AAA |
| 33 | GGQGGQGGYGGLGSQGAGQGGYGQGGVAAAAAAASGAGGAGRGGLGAGGAGQEYGAV |
| | SGGQGGAGQGGEAAAAAAAAGGQGGQGGYGGLGSQGAGQGGYGQGGAAAAAAAASGA |
| | GGARRGGLGAGGAGQGYGAGLGGQGGAGQGSASAAAAAAAGGQGGQGGYGGLGSQGS |
| | GQGGYGCGGAAAAAAAASGAGGAGRGSLGAGGAGQGYGAGLGGQGGAGQGGAAAAAS |
| | AAA |
| 34 | GPGGYGGPGQQGPGQGQYGPGTGQQGQGPGGQQGPVGAAAAAAAAVSSGGYGSQGAG |
| | QGGQQGSGQRGPAAAGPGGYSGPGQQGPGQGGQOGPASAAAAAAAAAGPGGYGGSGQ |
| | QGPGQGRGTGQQGQGPGGQQGPASAAAAAAAGPGGYGGPGQQGPGQGQYGPGTGQQG |
| | QGPASAAAAAAAGPGGYGGPGQQGPGQGQYGPGTGQQGQGPGGQQGPGGASAAAAAA |
| | A |
| 35 | GGYGPGAGQQGPGSGGQQGPGGQGPYGSGQQGPGGAGQQGPGGQGPYGPGAAAAAAA |
| | AAGGYGPGAGQQGPGGAGQQGPGSQGPGGQGPYGPGAGQQGPGSQGPGSGGQQGPGG |
| | QGPYGPSAAAAAAAAAGGYGPGAGQRSQGPGGQGPYGPGAGQQGPGSQGPGSGGQQG |
| | PGGQGPYGPSAAAAAAAAGPGAGRQGPGSQGPGSGGQQGPGGQGPYGPSAAAAAAAA |
| 36 | GQGGQGGQGGLGQGGYGQGAGSSAAAAAAAAAAAAAAGRGQGGYGQGSGGNAAAAAA |
| | AAAAAASGQGSQGGQGGQGQGGYGQGAGSSAAAAAAAAAAAAASGRGQGGYGQGAGG |
| | NAAAAAAAAAAAAAAGQGGQGGYGGLGQGGYGQGAGSSAAAAAAAAAAAAGGQGGQG |
| | QGGYGQGSGGSAAAAAAAAAAAAAAAGRGQGGYGQGSGGNAAAAAAAAAAAAAA |
| 37 | GRGPGGYGPGQQGPGGPGAAAAAAGPGGYGPGGYGPGQQGPGGPGAAAAAAAGRGPG |
| | GYGPGQQGPGQQGPGGSGAAAAAAGRGPGGYGPGQOGPGGPGAAAAAAGPGGYGPGQ |
| | QGPGAAAAAAAAGRGPGGYGPGGQGPGGPGAAAAAAAGRGPGGYGPGQQGPGQQGPG |
| | GSGAAAAAAGRGPGGYGPGQQGPGGPGAAAAAAGPGGYGPGQQGPGAAAAAAAA |
| 38 | GRGPGGYGPGQQGPGGSGAAAAAAGRGPGGYGPGQQGPGGPGAAAAAAGPGGYGPGQ |
| | QGTGAAAAAAAGSGAGGYGPGQQGPGGPGAAAAAAGPGGYGPGQQGPGAAAAAAAGS |
| | GPGGYGPGQQGPGGSSAAAAAAGPGRYGPGQQGPGAAAAASAGRGPGGYGPGQQGPG |
| | GPGAAAAAAGPGGYGPGQQGPGAAAAAAAGSGPGGYGPGQQGPGGPGAAAAAAA |
| 39 | GAAATAGAGASVAGGYGGGAGAAAGAGAGGYGGGYGAVAGSGAGAAAAASSGAGGAA |
| | GYGRGYGAGSGAGAGAGTVAAYGGAGGVATSSSSATASGSRIVTSGGYGYGTSAAAG |
| | AGVAAGSYAGAVNRLSSAEAASRVSSNIAAIASGGASALPSVISNIYSGVVASGVSS |
| | NEALIQALLELLSALVHVLSSASIGNVSSVGVDSTLNVVQDSVGQYVG |
| 40 | GGQGGFSGQGQGGFGPGAGSSAAAAAAAAAAARQGGQGQGGFGQGAGGNAAAAAAAA |
| | AAAAAAQQGGQGGFSGRGQGGFGPGAGSSAAAAAAGQGGQGQGGFGQGAGGNAAAAA |
| | AAAAAAAAAAGQGGQGRGGFGQGAGGNAAAAAAAAAAAAAAAQQGGQGGFGGRGQGG |
| | FGPGAGSSAAAAAAGQGGQGRGGFGQGAGGNAAAASAAAAASAAAAGQ |
| 41 | GGYGPGAGQQGPGGAGQQGPGSQGPGGAGQQGPGGQGFYGPGAAAAAAAVGGYGPGA |
| | GQQGPGSQGPGSGGQQGPGGQGPYGPSAAAAAAAAGGYGPGAGQQGPGSQGPGSGGQ |
| | QGPGGLGPYGPSAAAAAAAAGGYGPGAGQQGPGSQGPGSGGQQRPGGLGPYGFSAAA |
| | AAAAAGGYGPGAGQQGPGSQGPGSGGQQRPGGLGPYGPSAAAAAAAA |
| 42 | GAGAGGGYGGGYSAGGGAGAGSGAAAGAGAGRGGAGGYSAGAGTGAGAAAGAGTAGG |
| | YSGGYGAGASSSAGSSFISSSSMSSSQATGYSSSSGYGGGAASAAAGAGAAAGGYGG |
| | GYGAGAGAGAAAASGATGRVANSLGAMASGGINALPGVFSNIFSQVSAASGGASGGA |
| | VLVQALTEVIALLLHILSSASIGNVSSQGLEGSMAIAQQAIGAYAG |
| 43 | GAGAGGAGGYAQGYGAGAGAGAGAGTGAGGAGGYGQGYGAGSGAGAGGAGGYGAGAG |
| | AGAGAGDASGYGQGYGDGAGAGAGAAAAAGAAAGARGAGGYGGGAGAGAGAGAGAAG |
| | GYGQGYGAGAGEGAGAGAGAGAVAGAGAAAAAGAGAGAGGAEGYGAGAGAGGAGGYG |
| | QSYGDGAAAAAGSGAGAGGSGGYGAGAGAGSGAGAAGGYGGGAGA |
| 44 | GPGGYGPGQQGPGGYGPGQQGPGRYGPGQQGPSGPGSAAAAAAGSGQQGPGGYGPRQ |
| | QGPGGYGQGQQGPSGPGSAAAASAAASAESGQQGPGGYGPGQQGPGGYGPGQQGPGG |
| | YGPGQQGPSGPGSAAAAAAAASGPGQQGPGGYGPGQQGPGGYGPGQQGPSGPGSAAA |
| | AAAAASGPGQQGPGGYGFGQQGPGGYGPGQQGLSGPGSAAAAAAA |
| 45 | GRGPGGYGQGQQGPGGPGAAAAAAGPGGYGPGQQGPGAAAAAAAGSGPGGYGPGQQG |
| | PGRSGAAAAAAAAGRGPGGYGPGQQGPGGPGAAAAAAGPGGYGPGQQGPGAAAAASA |
| | GRGPGGYGPGQQGPGGSGAAAAAAGRGPGGYGPGQQGPGGPGAAAAAAAGRGPGGYG |
| | PGQQGPGQQGPGGSGAAAAAAGRGPGGYGPGQQGPGGFGAAAAAA |
| 46 | GVGAGGEGGYDQGYGAGAGAGSGGGAGGAGGYGGGAGAGSGGGAGGAGGYGGGAGAG |
| | AGAGAGGAGGYGGGAGAGTGARAGAGGVGGYGQSYGAGASAAAGAGVGAGGAGAGGA |
| | GGYGQGYGAGAGIGAGDAGGYGGGAGAGASAGAGGYGGGAGAGAGGVGGYGKGYGAG |
| | SGAGAAAAAGAGAGSAGGYGRGDGAGAGSASGYGQGYGAGAAA |
| 47 | GYGAGAGRGYGAGAGAGAGAVAASGAGAGAGYGAGAGAGAGAGYGAGAGRGYGAGAG |
| | AGAGSGAASGAGAGAGYGAGAGAGAGYGAGAGSGYGTGAGAGAGAAAAGGAGAGAGY |
| | GAGAGRGYGAGAGAGAASGAGAGAGAGAASGAGAGSGYGAGAAAAGGAGAGAGGGYG |
| | AGAGRGYGAGAGAGAGAGSGSGSAAGYGQGYGSGSGAGAAA |
| 48 | GQGTDSSASSVSTSTSVSSSATGPDTGYPVGYYGAGQAEAAASAAAAAAASAAEAAT |
| | IAGLGYGRQGQGTDSSASSVSTSTSVSSSATGPDMGYPVGNYGAGQAEAAASAAAAA |
| | AASAAEAATIASLGYGRQGQGTDSSASSVSTSTSVSSSATGPGSRYPVRDYGADQAE |
| | AAASAAAAAAAAASAAEEIASLGYGRQ |
| 49 | GQGTDSVASSASSSASASSSATGPDTGYPVGYYGAGQAEAAASAAAAAAASAAEAAT |
| | IAGLGYGRQGQGTDSSASSVSTSTSVSSSATGPGSRYPVRDYGADQAEAAASATAAA |
| | AAAASAAEEIASLGYGRQGQGTDSVASSASSSASASSSATGPDTGYPVGYYGAGQAE |
| | AAASAAAAAAASAAEAATIAGLGYGRQ |
| 50 | GQGGQGGYGGLGQGGYGQGAGSSAAAAAAAAAAAAAGGQGGQGQGRYGQGAGSSAAA |
| | AAAAAAAAAAAGRGQGGYGQGSGGNAAAAAAAAAAAASGQGSQGGQGGQGQGGYGQG |
| | AGSSAAAAAAAAAAAAASGRGQGGYGQGAGGNAAAAAAAAAAAAAAGQGGQGGYGGL |
| | GQGGYGQGAGSSAAAAAAAAAAAA |
| 51 | GGLGGQGGLGGLGSQGAGLGGYGQGGAGQGGAAAAAAAAGGLGGQGGRGGLGSQGAG |
| | QGGYGQGGAGQGGAAAAAAAAGGLGGQGGLGALGSQGAGQGGAGQGGYGQGGAAAAA |
| | AGGLGGQGGLGGLGSQGAGQGGYGQGGAGQGGAAAAAAAAGGLGGQGGLGGLGSQGA |
| | GPGGYGQGGAGQGGAAAAAAAA |
| 52 | GGQGRGGFGQGAGGNAAAAAAAAAAAAAAQQVGQFGFGGRGQGGFGPFAGSSAAAAA |
| | AASAAAGQGGQGQGGFGQGAGGNAAAAAAAAAAAARQGGQGQGGFSQGAGGNAAAAA |
| | AAAAAAAAAAQQGGQGGFGGRGQGGFGPGAGSSAAAAAAATAAAGQGGQGRGGFGQG |
| | AGSNAAAAAAAAAAAAAAAGQ |
| 53 | GGQGGQGGYGGLGSQGAGQGGYGAGQGAAAAAAAAGGAGGAGRGGLGAGGAGQGYGA |
| | GLGGQGGAGQAAAAAAAGGAGGARQGGLGAGGAGQGYGAGLGGQGGAGQGGAAAAAA |
| | AAGGQGGQGGYGGLGSQGAGQGGYGAGQGGAAAAAAAAGGQGGQGGYGGLGSQGAGQ |
| | GGYGGRQGGAGAAAAAAAA |
| 54 | GGAGQRGYGGLGNQGAGRGGLGGQGAGAAAAAAAGGAGQGGYGGLGNQGAGRGGQGA |
| | AAAAGGAGQGGYGGLGSQGAGRGGQGAGAAAAAAVGAGQEGIRGQGAGQGGYGGLGS |
| | QGSGRGGLGGQGAGAAAAAAGGAGQGGLGGQGAGQGAGAAAAAAGGVRQGGYGGLGS |
| | QGAGRGGQGAGAAAAAA |
| 55 | GGAGQGGLGGQGAGQGAGASAAAAGGAGQGGYGGLGSQGAGRGGEGAGAAAAAAGGA |
| | GQGGYGGLGGQGAGQGGYGGLGSQGAGRGGLGGQGAGAAAAGGAGQGGLGGQGAGQG |
| | AGAAAAAAGGAGQGGYGGLGSQGAGRGGLGGQGAGAVAAAAAGGAGQGGYGGLGSQG |
| | AGRGGQGAGAAAAAA |
| 56 | GAGAGAGAGSGAGAAGGYGGGAGAGVGAGGAGGYDQGYGAGAGAGSGAGAGGAGGYG |
| | GGAGAGADAGAGGAGGYGGGAGAGAGARAGAGGVGGYGQSYGAGAGAGAGVGAGGAG |
| | AGGADGYGQGYGAGAGTGAGDAGGYGGGAGAGASAGAGGYGGGAGAGGVGVYGKGYG |
| | SGSGAGAAAAA |
| 57 | GGAGGYGVGOGYGAGAGAGAAAGAGAGGAGGYGAGQGYGAGAGVGAAAAAGAGAGVG |
| | GAGGYGRGAGAGAGAGAGAAAGAGAGAAAGAGAGGAGGYGAGQGYGAGAGVGAAAAA |
| | GAGAGVGGAGGYGRGAGAGAGAGAGGAGGYGRGAGAGAGAGAGAGGAGGYGAGQGYG |
| | AGAGAGAAAAA |
| 58 | GEAFSASSASSAVVFESAGPGEEAGSSGDGASAAASAAAAAGAGSGRRGPGGARSRG |
| | GAGAGAGAGSGVGGYGSGSGAGAGAGAGAGAGGEGGFGEGQGYGAGAGAGFGSGAGA |
| | GAGAGSGAGAGEGVGSGAGAGAGAGFGVGAGAGAGAGAGFGSGAGAGSGAGAGYGAG |
| | RAGGRGRGGRG |
| 59 | GEAFSASSASSAVVFESAGPGEEAGSSGGGASAAASAAAAAGAGSGRRGPGGARSRG |
| | GAGAGAGAGSGVGGYGSGSGAGAGAGAGAGAGGEGGFGEGQGYGAGAGAGFGSGAGA |
| | GAGAGSGAGAGEGVGSGAGAGAGAGFGVGAGAGAGAGAGFGSGAGAGSGAGAGYGAG |
| | RAGGRGRGGRG |
| 60 | GNGLGQALLANGVLNSGNYLQLANSLAYSFGSSLSQYSSSAAGASAAGAASGAAGAG |
| | AGAASSGGSSGSASSSTTTTTTTSTSAAAAAAAAAAAASAAASTSASASASASASAS |
| | AFSQTFVQTVLQSAAFGSYFGGNLSLQSAQAAASAAAQAAAQQIGLGSYGYALANAV |
| | ASAFASAGANA |
| 61 | GNGLGQALLANGVLNSGNYLQLANSLAYSFGSSLSQYSSSAAGASAAGAASGAAGAG |
| | AGAASSGGSSGSASSSTTTTTTTSTSAAAAAAAAAAAASAAASTSASASASASASAS |
| | AFSQTFVQTVLQSAAFGSYFGGNLSLQSAQAAASAAAQAAAQQIGLGSYGYALANAV |
| | ASAFASAGANA |
| 62 | GNGLGQALLANGVLNSGNYLQLANSLAYSFGSSLSQYSS3AAGASAAGAASGAAGAG |
| | AGAASSGGSSGSASSSTTTTTTTSTSAAAAAAAAAAAASAAASTSASASASASASAS |
| | AFSQTFVQTVLQSAAFGSYFGGNLSLQSAQAAASAAAQAAAQQIGLGSYGYALANAV |
| | ASAFASAGANA |
| 63 | GASGAGQGQGYGQQGQGGSSAAAAAAAAAAAAAAAQGQGQGYGQQGQGSAAAAAAAA |
| | AAGASGAGQGQGYGQQGQGSAAAAAAAAAAGASGAGQGQGYGQQGQGGSSAAAAAAA |
| | AAAAAAAAAQGQGYGQQGQGSAAAAAAAAAGASGAGQGQGYGQQGQGGSSAAAAAAA |
| | AAAAAAAAA |
| 64 | GRGQGGYGQGSGGNAAAAAAAGQGGFGGQEGNGQGAGSAAAAAAAAAAAAGGSGQGR |
| | YGGRGQGGYGQGAGAAASAAAAAAAAAAGQGGFGGQEGNGQGAGSAAAAAAAAAAAA |
| | GGSGQGGYGGRGQGGYGQGAGAAAAAAAAAAAAAAGQGGQGGFGSQGGNGQGAGSAA |
| | AAAAAAAA |
| 65 | GQNTPWSSTELADAFINAFMNEAGRTGAFTADQLDDMSTIGDTIKTAMDKMARSNKS |
| | SKGKLQALNMAFASSMAEIAAVEQGGLSVDAKTNAIADSLNSAFYQTTGAANPQFVN |
| | EIRSLINMFAQSSANEVSYGGGYGGQSAGAAASAAAAGGGGQGGYGNLGGQGAGAAA |
| | AAAASAA |
| 66 | GQNTPWSSTELADAFINAFLNEAGRTGAFTADQLDDMSTIGDTLKTAMDKMARSNKS |
| | SQSKLQALNMAFASSMAEIAAVEQGGLSVAEKTNAIADSLNSAFYQTTGAVNVQFVN |
| | EIRSLISMFAQASANEVSYGGGYGGGQGGQSAGAAAAAASAGAGQGGYGGLGGQGAG |
| | SAAAAAA |
| 67 | GGQGGQGGYGGLGSQGAGQGGYGQGGAAAAAASAGGQGGQGGYGGLGSQGAGQGGYG |
| | GGAFSGQQGGAASVATASAAASRLSSPGAASRVSSAVTSLVSSGGPTNSAALSNTIS |
| | NVVSQISSSNPGLSGCDVLVQALLEIVSALVHILGSANIGQVNSSGVGRSASIVGQS |
| | INQAFS |
| 68 | GGAGQGGYGGLGGQGAGAAAAAAGGAGQGGYGGQGAGQGAAAAAASGAGQGGYEGPG |
| | AGQGAGAAAAAAGGAGQGGYGGLGGQGAGQGAGAAAAAAGGAGQGGYGGLGGQGAGQ |
| | GAGAAAAAAGGAGQGGYGGQGAGQGAAAAAAGGAGQGGYGGLGSGQGGYGRQGAGAA |
| | AAAAAA |
| 69 | GASSAAAAAAATATSGGAPGGYGGYGPGIGGAFVPASTTGTGSGSGSGAGAAGSGGL |
| | GGLGSSGGSGGLGGGNGGSGASAAASAAAASSSPGSGGYGPGQGVGSGSGSGAAGGS |
| | GTGSGAGGPGSGGYGGPQFFASAYGGQGLLGTSGYGNGQGGASGTGSGGVGGSGSGA |
| | GSNS |
| 70 | GQPIWTNPNAAMTMTNNLVQCASRSGVLTADQMDDMGMMADSVNSQMQKMGPNPPQH |
| | RLRAMNTAMAAEVAEVVATSPPQSYSAVLNTIGACLRESMMQATGSVDNAFTNEVMQ |
| | LVKMLSADSANEVSTASASGASYATSTSSAVSSSQATGYSTAAGYGNAAGAGAGAAA |
| | AVS |
| 71 | GQKIWTNPDAAMAMTNNLVQCAGRSGALTADQMDDLGMVSDSVNSQVRKMGANAPPH |
| | KIKAMSTAVAAGVAEVVASSPPQSYSAVLNTIGGCLRESMMQVTGSVDNTFTTEMMQ |
| | MVNMFAADNANEVSASASGSGASYATGTSSAVSTSQATGYSTAGGYGTAAGAGAGAA |
| | AAA |
| 72 | GSGYGAGAGAGAGSGYGAGAGAGSGYGAGAGAGAGSGYVAGAGAGAGAGSGYGAGAG |
| | AGAGSSYSAGAGAGAGSGYGAGSSASAGSAVSTQTVSSSATTSSQSAAAATGAAYGT |
| | RASTGSGASAGAAASGAGAGYGGQAGYGQGGGAAAYRAGAGSQAAYGQGASGSSGAA |
| | AAA |
| 73 | GGQGGRGGFGGLSSQGAGGAGQGGSGAAAAAAAAGGDGGSGLGDYGAGRGYGAGLGG |
| | AGGAGVASAAASAAASRLSSPSAASRVSSAVTSLISGGGPTNPAALSNTFSNWYQI |
| | SVSSPGLSGCDVLIQALLELVSALVHILGSAIIGQVNSSAAGESASLVGQSVYQAFS |
| 74 | GVGQAATPWENSQLAEDFINSFLRFIAQSGAFSPNQLDDMSSIGDTLKTAIEKMAQS |
| | RKSSKSKLQALNMAFASSHAEIAVAEQGGLSLEAKTNAIANALASAFLETTGFVNQQ |
| | FVSEIKSLIYMIAQASSNEISGSAAAAGGGSGGGGGSGQGGYGQGASASASAAAA |
| 75 | GGGDGYGQGGYGNQRGVGSYGQGAGAGAAATSAAGGAGSGRGGYGEQGGLGGYGQGA |
| | GAGAASTAAGGGDGYGQGGYGNQGGRGSYGQGSGAGAGAAVAAAAGGAVSGQGGYDG |
| | EGGQGGYGQGSGAGAAVAAASGGTGAGQGGYGSQGSQAGYGQGAGFRAAAATAAA |
| 76 | GAGAGYGGQVGYGQGAGASAGAAAAGAGAGYGGQAGYGQGAGGSAGAAAAGAGAGRQ |
| | AGYGQGAGASARAAAAGAGTGYGQGAGASAGAAAAGAGAGSQVGYGQGAGASSGAAA |
| | AAGAGAGYGGQVGYEQGAGASAGAEAAASSAGAGYGGQAGYGQGAGASAGAAAA |
| 77 | GGAGQGGYGGLGGQGAGQGGLGGQRAGAAAAAAGGAGQGGYGGLGSQGAGRGGYGGV |
| | GSGASAASAAASRLSSPEASSRVSSAVSNLVSSGPTNSAALSSTISNVVSQISASNP |
| | GLSGCDVLVQALLEVVSALIQILGSSSIGQVNYGTAGQAAQIVGQSVYQALG |
| 78 | GGYGPGSGQQGPGGAGQQGPGGQGPYGPGSSSAAAVGGYGPSSGLQGPAGQGPYGPG |
| | AAASAAAAAGASRLSSPQASSRVSSAVSSLVSSGPTNSAALTNTISSVVSQISASNP |
| | GLSGCDVLIQALLEIVSALVHILGYSSIGQINYDAAAQYASLVGQSVAQALA |
| 79 | GGAGAGQGSYGGQGGYGQGGAGAATATAAAAGGAGSGQGGYGGQGGLGGYGQGAGAG |
| | AAAAAAAAAGGAGAGQGGYGGQGGQGGYGQGAGAGAAAAAAGGAGAGQGGYGGQGGY |
| | GQGGGAGAAAAAAAASGGSGSGQGGYGGQGGLGGYGQGAGAGAGAAASAAAA |
| 80 | GQGGQGGYGRQSQGAGSAAAAAAAAAAAAAAGSGQGGYGGQGQGGYGQSSASASAAA |
| | SAASTVANSVSRLSSPSAVSRVSSAVSSLVSNGQVNMAALPNIISNISSSVSASAPG |
| | ASGCEVIVQALLEVITALVQIVSSSSVGYINPSAVNQITNVVANAMAQVMG |
| 81 | GGAGQGGYGGLGGQGSGAAAAGTGQGGYGSLGGQGAGAAGAAAAAVGGAGQGGYGGV |
| | GSAAASAAASRLSSPEASSRVSSAVSNLVSSGPTNSAALSNTISNVVSQISSSNPGL |
| | SGCDVLVQALLEWSALIHILGSSSIGQVNYGSAGQATQIVGQSVYQALG |
| 82 | GAGAGGAGGYGAGQGYGAGAGAGAAAGAGAGGARGYGARQGYGSGAGAGAGARAGGA |
| | GGYGRGAGAGAAAASGAGAGGYGAGQGYGAGAGAVASAAAGAGSGAGGAGGYGRGAG |
| | AVAGAGAGGAGGYGAGAGAAAGVGAGGSGGYGGRQGGYSAGAGAGAAAAA |
| 83 | GQGGQGGYGGLGQGGYGQGAGSSAAAAAAAAAAAGRGQGGYGQGSGGNAAAAAAAAA |
| | AAASGQGGQGGQGGQGQGGYGQGAGSSAAAAAAAAAAAAAAAGRGQGGYGQGAGGNA |
| | AAAAAAAAAAASGQGGQGGQGGQGQGGYGQGAGSSAAAAAAAAAAAAAA |
| 84 | GGYGPGSGQQGPGQQGPGQQGPGQQGPYGAGASAAAAAAGGYGPGSGQQGPGVRVAA |
| | PVASAAASRLSSSAASSRVSSAVSSLVSSGPTTPAALSNTISSAVSQISASNPGLSG |
| | CDVLVQALLEVVSALVHILGSSSVGQINYGASAQYAQMVGQSVTQALV |
| 85 | GAGAGGAGYGRGAGAGAGAAAGAGAGAAAGAGAGAGGYGGQGGYGAGAGAGAAAAAG |
| | AGAGGAAGYSRGGRAGAAGAGAGAAAGAGAGAGGYGGQGGYGAGAGAGAAAAAGAGS |
| | GGAGGYGRGAGAGAAAGAGAAAGAGAGAGGYGGQGGYGAGAGAAAAA |
| 86 | GAGAGRGGYGRGAGAGGYGGQGGYGAGAGAGAAAAAGAGAGGYGDKEIACWSRCRYT |
| | VASTTSRLSSAEASSRISSAASTLVSGGYLNTAALPSVISDLFAQVGASSPGVSDSE |
| | VLIQVLLEIVSSLIHILSSSSVGQVDFSSVGSSAAAVGQSMQVVMG |
| 87 | GAGAGAGGAGGYGRGAGAGAGAGAGAAAGQGYGSGAGAGAGASAGGAGSYGRGAGAG |
| | AAAASGAGAGGYGAGQGYGAGAGAVASAAAGAGSGAGGAGGYGRGAVAGSGAGAGAG |
| | AGGAGGYGAGAGAGAAAGAVAGGSGGYGGRQGGYSAGAGAGAAAAA |
| 88 | GPGGYGPVQQGPSGPGSAAGPGGYGPAQQGPARYGPGSAAAAAAAAGSAGYGPGPQA |
| | SAAASRLASPDSGARVASAVSNLVSSGPTSSAALSSVISNAVSQIGASNPGLSGCDV |
| | LIQALLEIVSACVTILSSSSIGQVNYGAASQFAQVVGQSVLSAFS |
| 89 | GTGGVGGLFLSSGDFGRGGAGAGAGAAAASAAAASSAAAGARGGSGFGVGTGGFGRG |
| | GAGAGTGAAAASAAAASAAAAGAGGDGGLFLSSGDFGRGGAGAGAGAAAASAAAASS |
| | AAAGARGGSGFGVGTGGFGRGGAGDGASAAAASAAAASAAAA |
| 90 | GGYGPGAGQQGPGGAGQQGPGGQGPYGPSVAAAASAAGGYGPGAGQQGFVASAAVSR |
| | LSSPQASSRVSSAVSSLVSSGPTNPAALSNAMSSVVSQVSASNPGLSGCDVLVQALL |
| | EIVSALVHILGSSSIGQINYAASSQYAQMVGQSVAQALA |
| 91 | GGAGQGGYGGLGSQGAGRGGYGGQGAGAAAAATGGAGQGGYGGVGSGASAASAAASR |
| | LSSPQASSRVSSAVSNLVASGPTNSAALSSTISNAVSQIGASNPGLSGCDVLIQALL |
| | EVVSALIHILGSSSIGQVNYGSAGQATQIVGQSVYQALG |
| 92 | GGAGQGGYGGLGSQGAGRGGYGGQGAGAAVAAIGGVGQGGYGGVGSGASAASAAASR |
| | LSSPEASSRVSSAVSNLVSSGPTNSAALSSTISNWSQIGASNPGLSGCDVLIQALL |
| | EVVSALVHILGSSSIGQVNYGSAGQATQIVGQSVYQALG |
| 93 | GASGGYGGGAGEGAGAAAAAGAGAGGAGGYGGGAGSGAGAVARAGAGGAGGYGSGIG |
| | GGYGSGAGAAAGAGAGGAGAYGGGYGTGAGAGARGADSAGAAAGYGGGVGTGTGSSA |
| | GYGRGAGAGAGAGAAAGSGAGAAGGYGGGYGAGAGAGA |
| 94 | GAGSGQGGYGGQGGLGGYGQGAGAGAAAGASGSGSGGAGQGGLGGYGQGAGAGAAAA |
| | AAGASGAGQGGFGPYGSSYQSSTSYSVTSQGAAGGLGGYGQGSGAGAAAAGAAGQGG |
| | QGGYGQGAGAGAGAGAGQGGLGGYGQGAGSSAASAAAA |
| 95 | GGAGQGGYGGLGGQGVGRGGLGGQGAGAAAAGGAGQGGYGGVGSGASAASAAASRLS |
| | SPQASSRLSSAVSNLVATGPTNSAALSSTISNVVSQIGASNPGLSGCDVLIQALLEV |
| | VSALIQILGSSSIGQVNYGSAGQATQIVGQSVYQALG |
| 96 | GAGSGGAGGYGRGAGAGAGAAAGAGAGAGSYGGQGGYGAGAGAGAAAAAGAGAGAGG |
| | YGRGAGAGAGAGAGAAARAGAGAGGAGYGGQGGYGAGAGAGAAAAAGAGAGGAGGYG |
| | RGAGAGAGAAAGAGAGAGGYGGQSGYGAGAGAAAAA |
| 97 | GASGAGQGQGYGQQGQGGSSAAAAAAAAAAAQGQGQGYGQQGQGYGQQGQGGSSAAA |
| | AAAAAAAAAAQGQGQGYGQQGQGSAAAAAAAAAGASGAGQGQGYGQQGQGGSSAAAA |
| | AAAAAAAAAAAQGQGYGQQGQGSAAAAAAAAAAAAA |
| 98 | GGYGPRYGQQGPGAGPYGPGAGATAAAAGGYGPGAGQQGPRSQAPVASAAAARLSSP |
| | QAGSRVSSAVSTLVSSGPTNPASLSNAIGSVVSQVSASNPGLPSCDVLVQALLEIVS |
| | ALVHILGSSSIGQINYSASSQYARLVGQSIAQALG |

In an embodiment a block copolymer polypeptide repeat unit that forms fibers with good mechanical properties is synthesized using SEQ ID NO. 1. This repeat unit contains 6 quasi-repeats, each of which includes motifs that vary in composition, as described herein. This repeat unit can be concatenated 1, 2, 3, 4, 5, 6, 7, 8, 9, 10, 11, 12, 13, 14, 15, 16, 17, 18, 19 or 20 times to form polypeptide molecules from 20 kDal to 535 kDal, or greater than 20 kDal, or greater than 10 kDal, or greater than 5 kDal, or from 5 to 400 kDal, or from 5 to 300 kDal, or from 5 to 200 kDal, or from 5 to 100 kDal, or from 5 to 50 kDal, or from 5 to 600 kDal, or from 5 to 800 kDal, or from 5 to 1000 kDal, or from 10 to 400 kDal, or from 10 to 300 kDal, or from 10 to 200 kDal, or from 10 to 100 kDal, or from 10 to 50 kDal, or from 10 to 600 kDal, or from 10 to 800 kDal, or from 10 to 1000 kDal, or from 20 to 400 kDal, or from 20 to 300 kDal, or from 20 to 200 kDal, or from 20 to 100 kDal, or from 20 to 50 kDal, or from 40 to 300 kDal, or from 40 to 500 kDal, or from 20 to 600 kDal, or from 20 to 800 kDal, or from 20 to 1000 kDal. This polypeptide repeat unit also contains poly-alanine regions related to nanocrystalline regions, and glycine-rich regions related to beta-turn containing less-crystalline regions. In other embodiments the repeat is selected from any of the sequences listed as Seq ID Nos: 2-97.

In some embodiments, the quasi-repeat unit of the polypeptide can be described by the formula {GGY-[GPG-X₁]ₙ₁-GPS-(A)ₙ₂} (SEQ ID NO: 108), where X₁ is independently selected from the group consisting of SGGQQ (SEQ ID NO: 100), GAGQQ (SEQ ID NO: 101), GQGPY (SEQ ID NO: 102), AGQQ (SEQ NO: 103) and SQ, n1 is a number from 4 to 8, and n2 is a number from 6 to 20. The repeat unit is composed of multiple quasi-repeat units. In additional embodiments, 3 “long” quasi repeats are followed by 3 “short” quasi-repeat units. As mentioned above, short quasi- repeat units are those in which n1=4 or 5. Long quasi-repeat units are defined as those in which n1=6, 7 or 8. In some embodiments, all of the short quasi-repeats have the same X₁ motifs in the same positions within each quasi-repeat unit of a repeat unit. In some embodiments, no more than 3 quasi-repeat units out of 6 share the same X₁ motifs.

In additional embodiments, a repeat unit is composed of quasi-repeat units that do not use the same X₁ more than two occurrences in a row within a repeat unit. In additional embodiments, a repeat unit is composed of quasi-repeat units where at least 1, 2, 3, 4, 5, 6, 7, 8, 9, 10, 11, 12, 13, 14, 15, 16, 17, 18, 19 or 20 of the quasi-repeats do not use the same X₁ more than 2 times in a single quasi-repeat unit of the repeat unit.

In some embodiments, the structure of fibers formed from the described polypeptides form beta-sheet structures, beta-turn structures, or alpha-helix structures. In some embodiments, the secondary, tertiary and quaternary protein structures of the formed fibers are described as having nanocrystalline beta-sheet regions, amorphous beta-turn regions, amorphous alpha helix regions, randomly spatially distributed nanocrystalline regions embedded in a non-crystalline matrix, or randomly oriented nanocrystalline regions embedded in a non-crystalline matrix.

In some embodiments, the polypeptides utilized to form fibers with mechanical properties as described herein include glycine-rich regions from 20 to 100 amino acids long concatenated with poly-alanine regions from 4 to 20 amino acids long (SEQ ID NO: 109). In some embodiments, polypeptides utilized to form fibers with good mechanical properties comprise 5-25% poly-alanine regions (from 4 to 20 poly-alanine residues (SEQ ID NO: 109)). In some embodiments, polypeptides utilized to form fibers with good mechanical properties comprise 25-50% glycine. In some embodiments, polypeptides utilized to form fibers with good mechanical properties comprise 15-35% GGX, where X is any amino acid. In some embodiments, polypeptides utilized to form fibers with good mechanical properties comprise 15-60% GPG. In some embodiments, polypeptides utilized to form fibers with good mechanical properties comprise 10-40% alanine. In some embodiments, polypeptides utilized to form fibers with good mechanical properties comprise 5-20% proline. In some embodiments, polypeptides utilized to form fibers with good mechanical properties comprise 10-50% beta-turns. In some embodiments, polypeptides utilized to form fibers with good mechanical properties comprise 10-50% alpha-helix composition. In some embodiments all of these compositional ranges will apply to the same polypeptide. In some embodiments two or more of these compositional ranges will apply to the same polypeptide.

### Fiber Spin Dope and Spinning Parameters

In some embodiments, a spin dope is synthesized containing proteins expressed from any of the polypeptides of the present disclosure. The spin dope is prepared using published techniques such as those found in WO2015042164 A2. In some embodiments, a fiber spinning solution was prepared by dissolving the purified and dried block copolymer polypeptide in a formic acid-based spinning solution, using standard techniques. Spin dopes were incubated at 35° C. on a rotational shaker for three days with occasional mixing. After three days, the spin dopes were centrifuged at 16000 rcf for 60 minutes and allowed to equilibrate to room temperature for at least two hours prior to spinning.

In an embodiment the fraction of protein that is at least some percentage (e.g., 80%) of the intended length is determined through quantitative analysis of the results of a size-separation process. In an embodiment, the size-separation process can include size-exclusion chromatography. In an embodiment, the size-separation process can include gel electrophoresis. The quantitative analysis can include determining the fraction of total protein falling within a designated size range by integrating the area of a chromatogram or densitometric scan peak. For example, if a sample is run through a size-separation process, and the relative areas under the peaks corresponding to full-length, 60% full-length and 20% full length are 3:2:1, then fraction that is full length corresponds to 3 parts out of a total of 6 parts=50%.

In some embodiments, the proteins of the spin dope, expressed from any of the polypeptides of the present disclosure, are substantially monodisperse, with >5%, or >10%, or >15%, or >20%, or >25%, or >30%, or >35%, or >40%, or >45%, or >50%, or >55%, or >60%, or >65%, or >70%, or >75% or >80%, or >85%, or >90%, or >95%, or >99% of the protein in the spin dope having molecular weight >5%, or >10%, or >15%, or >20%, or >25%, or >30%, or >35%, or >40%, or >45%, or >50%, or >55%, or >60%, or >65%, or >70%, or >75%, or >80%, or >85%, or >90%, or >95%, or >99% of the molecular weight of the encoded proteins. In some embodiments, the proteins of the spin dope, expressed from any of the polypeptides of the present disclosure, have from 5% to 99%, or from 5% to 50%, or from 50% to 99%, or from 20% to 80%, or from 40% to 60%, or from 5% to 30%, or from 70% to 99%, or from 5% to 20%, or from 5% to 10%, or from 80% to 99%, or from 90% to 99% of the protein in the spin dope having molecular weight from 5% to 99%, or from 5% to 50%, or from 50% to 99%, or from 20% to 80%, or from 40% to 60%, or from 5% to 30%, or from 70% to 99%, or from 5% to 20%, or from 5% to 10%, or from 80% to 99%, or from 90% to 99% of the molecular weight of the encoded proteins. The “encoded proteins” are defined as the polypeptide amino acid sequences that are encoded by the DNA utilized in protein expression. In other words, the “encoded proteins” are the polypeptides that would be produced if there were no imperfect processes (e.g. transcription errors, protein degradation, homologous recombination, truncation, protein fragmentation, protein agglomeration) at any stage during protein production. A higher monodispersity of proteins in the spin dopes, in other words a higher purity, has the advantage of producing fibers with better mechanical properties, such as higher Young's modulus, higher extensibility, higher ultimate tensile strength, and higher maximum tensile strength.

In one embodiment, 31% of the protein in the spin dope has molecular weight greater than 80% of the proteins that were intended to be produced (i.e., the encoded proteins). In this case, 70% of the proteins in the spin dope would be proteins other than the ones that were intended to be produced. One example of these other proteins are degraded protein fragments of the encoded proteins. Another example of these other proteins are foreign proteins that were not removed during any purification processes, such as proteins from the organisms being used to express the encoded proteins.

In other embodiments, fibers with low monodispersity, <5%, or <10%, or <15%, or <20%, or <25%, or <30%, or <35%, or <40%, or <45%, or <50%, or <55%, or <60%, or <65%, or <70%, or <75%, or <80%, or <85%, or <90%, or <95%, or <99% of the protein in the spin dope having molecular weight >5%, or >10%, or >15%, or >20%, or >25%, or >30%, or >35%, or >40%, or >45%, or >50%, or >55%, or >60%, or >65%, or >70%, or >75%, or >80%, or >85%, or >90%, or >95%, or >99% of the molecular weight of the proteins encoded by the DNA utilized in protein expression, were still able to create fibers with good mechanical properties. In other embodiments, fibers with low monodispersity, have from 5% to 99%, or from 5% to 50%, or from 5% to 30%, or from 10% to 50%, or from 20% to 50%, or from 50% to 99%, or from 20% to 80%, or from 40% to 60%, or from 5% to 30%, or from 70% to 99%, or from 5% to 20%, or from 5% to 10%, or from 80% to 99%, or from 90% to 99% of the protein in the spin dope having molecular weight 5% to 99%, or from 57o to 50%, or from 50% to 99%, or from 20% to 80%, or from 40% to 60%, or from 5% to 30%, or from 70% to 99%, or from 5% to 20%, or from 5% to 10%, or from 80% to 99%, or from 90% to 99% of the molecular weight of the proteins encoded by the DNA utilized in protein expression, were still able to create fibers with good mechanical properties. The mechanical properties described herein (e.g., high Young's (i.e., Elastic) modulus and/or extensibility (i.e., percent strain)), from fibers formed from low purity spin dopes was achieved through the use of the long polypeptide repeat units, suitable polypeptide compositions and spin dope and fiber spinning parameters described elsewhere in the present disclosure.

In other embodiments, the proteins are produced via secretion from a microorganism such as Pichia pastoris, Escherichia coli, Bacillus subtilis, or mammalian cells. Optionally, the secretion rate is at least 20 mg/g DCW/hr (DCW=dry cell weight). Optionally, the proteins are then recovered, separated, and spun into fibers using spin dopes containing solvents. Some examples of the classes of solvents that can be used in spin dopes are aqueous, inorganic or organic, including but not limited to ethanol, methanol, isopropanol, t-butyl alcohol, ethyl acetate, and ethylene glycol. Various methods for synthesizing recombinant proteinaceous block copolymers have been published such as those found in WO2015042164 A2.

In other embodiments, the coagulation bath conditions for wet spinning are chosen to promote fiber formation with certain mechanical properties. Optionally, the coagulation bath is maintained at temperatures of 0-90° C., more preferably 20-60° C. Optionally, the coagulation bath comprises about 60%, 70%, 80%, 90%, or even 100% alcohol, preferably isopropanol, ethanol, or methanol. Optionally, the coagulation bath is 95:5%, 90:10%, 85:15%, 80:20%, 75:25%, 70:30%, 65:35%, 60:40%, 55:45% or 50:50% by volume methanol:water. Optionally, the coagulation bath contains additives to enhance the fiber mechanical properties, such as additives comprising ammonium sulfate, sodium chloride, sodium sulfate, or other protein precipitating salts at temperature from 20 to 60° C.

In some embodiments, the extruded filament or fiber is passed through more than one bath. For embodiments in which more than one bath is used, the different baths have either different or same chemical compositions. In some embodiments, the extruded filament or fiber is passed through more than one coagulation bath. For embodiments in which more than one coagulation bath is used, the different coagulation baths have either different or same chemical compositions. The residence time can be tuned to improve mechanical properties, such as from 2 seconds to 100 minutes in the coagulant bath. The reeling/drawing rate can be tuned to improve fiber mechanical properties, such as a rate from 0.1 to 100 meters/minute.

The draw ratio can also be tuned to improve fiber mechanical properties. In different embodiments the draw ratio was 1.5× to 30×. In one embodiment, lower draw ratios improved the fiber extensibility. In one embodiment, higher draw ratios improved the fiber maximum tensile strength. Drawing can also be done in different environments, such as in solution, in humid air, or at elevated temperatures.

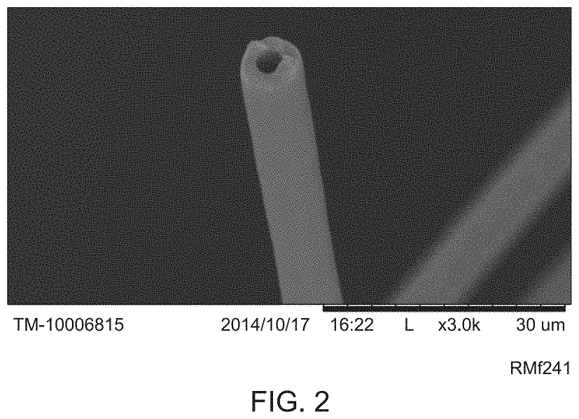

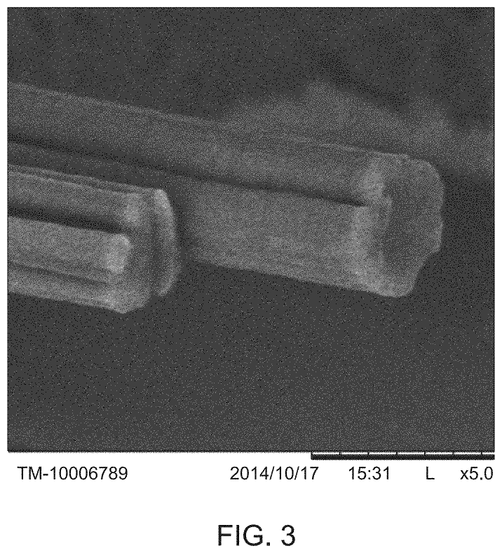

The fibers of the present disclosure processed with residence times in coagulation baths at the longer end of the disclosed range produce corrugated cross sections, as shown in FIG. 3. That is, each fiber has a plurality of corrugations (or alternatively “grooves”) disposed at an outer surface of a fiber. Each of these corrugations is parallel to a longitudinal axis of the corresponding fiber on which the corrugations are disposed. The fibers of the present disclosure processed with higher ethanol content in the coagulation bath produce hollow core fibers, as shown in FIG. 2. That is, the fiber includes an inner surface and an outer surface. The inner surface defines a hollow core parallel to the longitudinal axis of the fiber.

In some embodiments a coagulation bath or the first coagulation bath is prepared using combinations of one or more of water, acids, solvents and salts, including but not limited to the following classes of chemicals of Brønsted-Lowry acids, Lewis acids, binary hydride acids, organic acids, metal cation acids, organic solvents, inorganic solvents, alkali metal salts, and alkaline earth metal salts. Some examples of acids used in the preparation of a coagulation bath or the first coagulation bath are dilute hydrochloric acid, dilute sulfuric acid, formic acid and acetic acid. Some examples of solvents that are used in the preparation of the first coagulation bath are ethanol, methanol, isopropanol, t-butyl alcohol, ethyl acetate, and ethylene glycol. Examples of salts used in the preparation of a coagulation bath or the first coagulation bath include LiCl, KCl, BeCl2, MgCl2, CaCl2, NaCl, ammonium sulfate, sodium sulfate, and other salts of nitrates, sulfates or phosphates.

In some embodiments, the chemical composition and extrusion parameters of a coagulation bath or the first coagulation bath are chosen so that the fiber remains translucent in a coagulation bath or the first coagulation bath. In some embodiments the chemical composition and extrusion parameters of a coagulation bath or the first coagulation bath are chosen to slow down the rate of coagulation of the fiber in a coagulation bath or the first coagulation bath, which improves the ability to draw the resulting fiber in subsequent drawing steps. In various embodiments, these subsequent drawing steps are done in different environments, including wet, dry, and humid air environments. Examples of wet environments include one or more additional baths or coagulation baths. In some embodiments, the fiber travels through one or more baths after the first coagulation bath. The one or more additional baths, or coagulation baths, are prepared, in embodiments, using combinations of one or more of water, acids, solvents and salts, including but not limited to the following classes of chemicals of Br♀nsted-Lowry acids, Lewis acids, binary hydride acids, organic acids, metal cation acids, organic solvents, inorganic solvents, alkali metal salts, and alkaline earth metal salts. Some examples of acids that are used in the preparation of the second baths or coagulant baths are dilute hydrochloric acid, dilute sulfuric acid, formic acid and acetic acid. Some examples of solvents that are used in the preparation of the second coagulant baths are ethanol, methanol, isopropanol, t-butyl alcohol, ethyl acetate, and ethylene glycol. Some examples of salts used in the preparation of a second bath or coagulation bath include LiCl, KCl, MgCl2, CaCl2, NaCl, ammonium sulfate, sodium sulfate, and other salts of nitrates, sulfates, or phosphates. In some embodiments, there are two coagulation baths, where the first coagulation bath has a different chemical composition than the second coagulation bath, and the second coagulation bath has a higher concentration of solvents than the first coagulation bath. In some embodiments, there are more than two coagulation baths, and the first coagulation bath has a different chemical composition than the second coagulation bath, and the second coagulation bath has a lower concentration of solvents than the first coagulation bath. In some embodiments, there are two baths, the first being a coagulation bath and the second being a wash bath. In some embodiments, the first coagulation bath has a different chemical composition than the second wash bath, and the second wash bath has a higher concentration of solvents than the first bath. In some embodiments, there are more than two baths, and the first bath has a different chemical composition than the second bath, and the second bath has a lower concentration of solvents than the first bath.

In some embodiments a spin dope is further prepared using combinations of one or more of water, acids, solvents and salts, including but not limited to the following classes of chemicals of Brønsted-Lowry acids, Lewis acids, binary hydride acids, organic acids, metal cation acids, organic solvents, inorganic solvents, alkali metal salts, and alkaline earth metal salts. Some examples of acids that are used in the preparation of spin dopes are dilute hydrochloric acid, dilute sulfuric acid, formic acid and acetic acid. Some examples of solvents that are used in the preparation of spin dopes are ethanol, methanol, isopropanol, t-butyl alcohol, ethyl acetate, and ethylene glycol. Some examples of salts that are used in the preparation of spin dopes are LiCl, KCl, MgCl2, CaCl2, NaCl, ammonium sulfate, sodium sulfate, and other salts of nitrates, sulfates or phosphates.

In some embodiments, a spinneret is chosen to enhance the fiber mechanical properties. The dimensions of the spinneret can be from 0.001 cm to 5 cm long, and from 25 to 35 gauge. In some embodiments, a spinneret includes multiple orifices to spin multiple fibers simultaneously. In some embodiments, the cross-section of a spinneret gradually tapers to the smallest diameter at the orifice, is straight-walled and then quickly tapers to the orifice, or includes multiple constrictions. An extrusion pressure of a spin dope from a spinneret can also be varied to affect the fiber mechanical properties in a range from 10 to 1000 psi. The interaction between fiber properties and extrusion pressure can be affected by spin dope viscosity, drawing/reeling rate, and coagulation bath chemistry.

The concentration of protein to solvent in the spin dope is also an important parameter. In some embodiments, the concentration of protein weight for weight is 20%, or 25%, or 30%, or 35%, or 40%, or 45% or 50%, or 55%, or from 20% to 55%, or from 20% to 40%, or from 30% to 40%, or from 30% to 55%, or from 30% to 50% in solution with solvents and other additives making up the remainder.

### EXAMPLE 1: FIBER SPINNING

Copolymers of the present disclosure were secreted from Pichia pastoris commonly used for the expression of recombinant DNA using published techniques, such as those described in WO2015042164 A2. In some embodiments, a secretion rate of at least 20 mg/g DCW/hr (DCW=dry cell weight) was observed. The secreted proteins were purified, dried, and dissolved in a formic acid-based spinning solvent, using standard techniques, to generate a homogenous spin dope.

The spin dope was extruded through a 50-200 μm diameter orifice with 2:1 ratio of length to diameter into a room temperature alcohol-based coagulation bath comprising 20% formic acid with a residence time of 28 seconds. Fibers were pulled out of the coagulation bath under tension, strung through a wash bath consisting of 100% alcohol drawn to 4 times their length, and subsequently allowed to dry.

### EXAMPLE 2: FIBER CROSS-SECTION

Using the above synthesis methods, morphology of extruded fibers was varied by adjusting various parameters of a coagulation bath. For example, hollow core fibers (as shown in FIG. 2) were synthesized by having a higher ethanol content of the coagulation bath, as described above. In another example, corrugated morphologies (as shown in FIG. 3) were produced by increasing residence time in a coagulation bath to in the range of 2-100 seconds.

The fibers of the present disclosure processed with residence times in coagulation baths at the longer end of the disclosed range tend to show corrugated cross-sections, as shown in FIG. 3 and as described above.

Fibers of the present disclosure processed with higher ethanol content in a coagulation bath include hollow cores, as shown in FIG. 2 and described above.

### EXAMPLE 3: FIBER MECHANICAL PROPERTIES

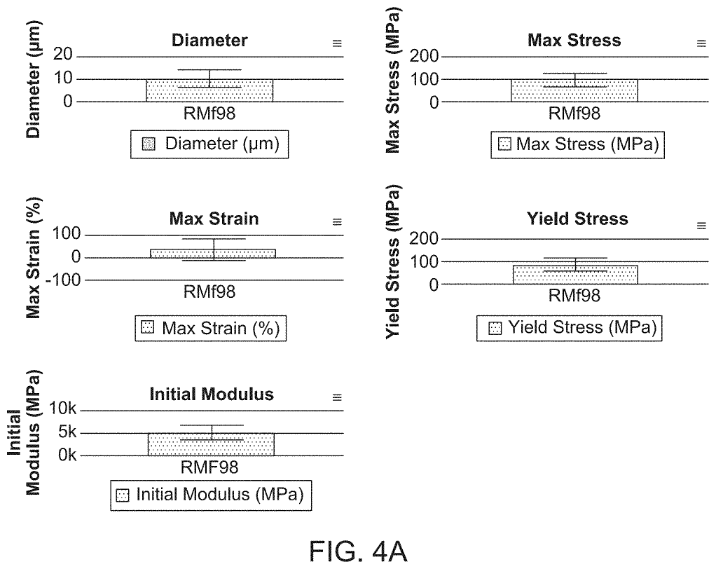

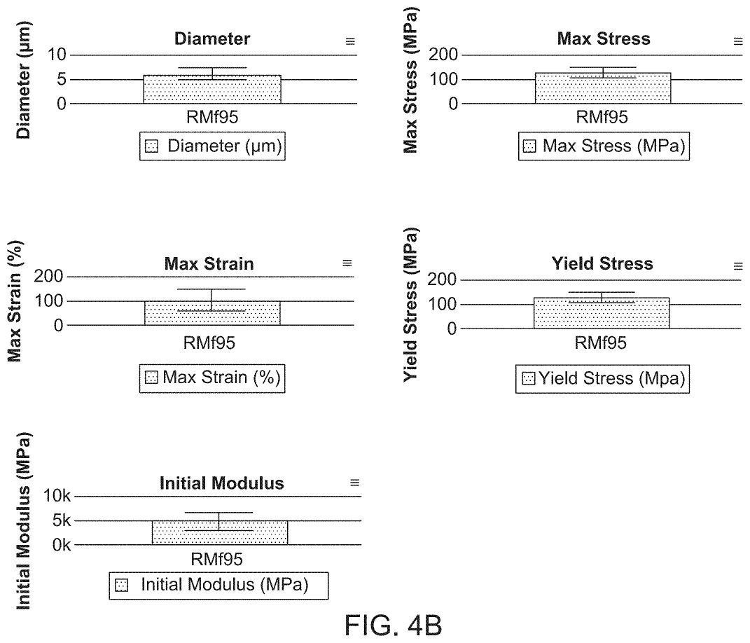

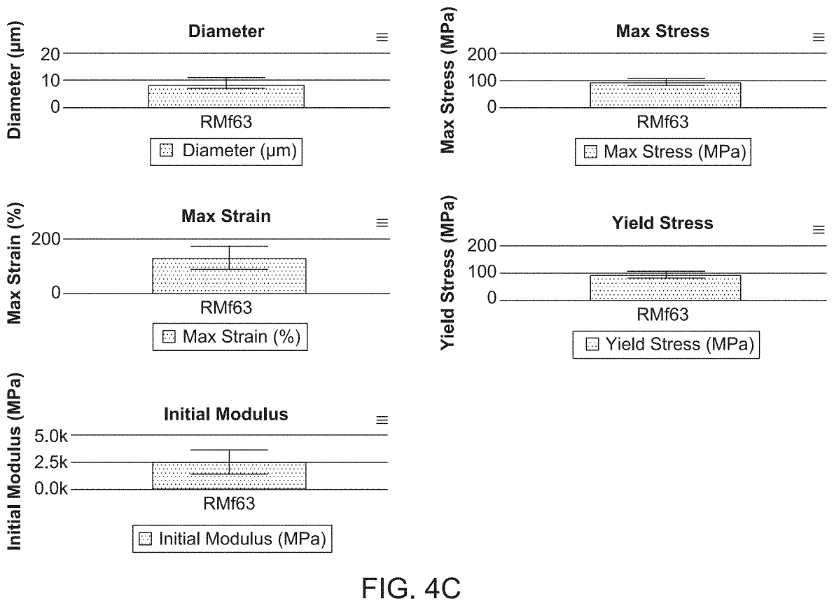

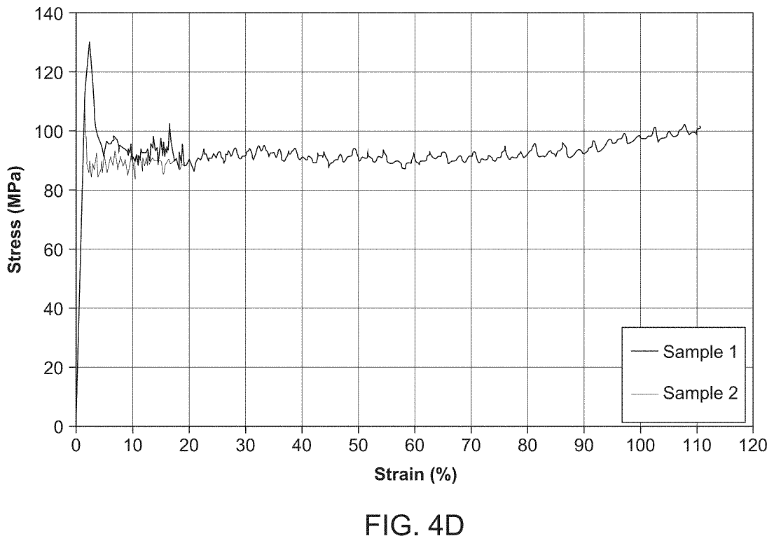

FIGS. 4A-4D and FIGS. 5-7 show various mechanical properties of measured samples, with the compositions described herein, and produced by the methods described herein.

Some of the mechanical properties of the fibers in this disclosure are reported in units of MPa (i.e. 10⁶ N/m², or force per unit area), and some are reported in units of cN/tex (force per linear density). The measurements of fibers mechanical properties reported in MPa were obtained using a custom instrument, which includes a linear actuator and calibrated load cell, and the fiber diameter was measured by light microscopy. The measurements of fibers mechanical properties reported in cN/tex were obtained using FAVIMAT testing equipment, which includes a measurement of the fiber linear density using a vibration method (e.g. according to ASTM D1577). To accurately convert measurements from MPa to cN/tex, an estimate of the bulk density (e.g. in g/cm³) of the fiber is used. An expression that can be used to convert a force per unit area in MPa, “FA”, to a force per linear density in cN/tex, “FLD”, using the bulk density in g/cm³, “BD”, is FLD=FA/(10*BD), Since the bulk density of recombinant silk can vary, a given value of fiber tenacity in MPa does not translate to a given value of fiber tenacity in cN/tex. However, if the bulk density of the recombinant silk is assumed to be from 1.1 to 1.4 g/cm³, then mechanical property values can be converted from one set of units into the other within a certain range of error. For example, a maximum tensile stress of 100 MPa is equivalent to about 9.1 cN/tex if the mass density of the fiber is 1.1 g/cm³, and a maximum tensile stress of 100 MPa is equivalent to about 7.1 cN/tex if the mass density of the fiber is 1.4 g/cm³.

A set of 4 fibers was tested for tensile mechanical properties using an instrument including a linear actuator and calibrated load cell, the results of which are shown in FIG. 4A. Fibers were pulled at 1% per second strain rate until failure. Fiber diameters were measured with light microscopy at 20× magnification using image processing software. The mean diameter was 10.25 um, +/−1 st.dev=6.4-14.1 um. The mean max tensile stress was 97.9 MPa, +/−1 st.dev=68.1-127.6 MPa. The mean max strain was 37.2%, +/−1 st.dev=−11.9-86.3%. The mean yield stress was 87.4 MPa, +/−1 st.dev=59.2-115.6 MPa. The mean initial modulus (same as elastic modulus, same as Young's modulus) was 5.2 GPa, +/−1 st.dev=3.5-6.9 GPa.

As shown in FIG. 4B, set of 7 fibers was tested for tensile mechanical properties using an instrument including a linear actuator and calibrated load cell. Fibers were pulled at 1% per second strain rate until failure. Fiber diameters were measured with light microscopy at 20× magnification using image processing software. The mean diameter was 6.2 um, +/−1 st.dev=4.9-7.5 um. The mean max tensile stress was 127.9 MPa, +/−1 st.dev=106.4-149.3 MPa. The mean max strain was 105.5%, +/−1 st.dev=61.0-150.0%. The mean yield stress was 109.8 MPa, +/−1 st.dev=91.4-128.2 MPa. The mean initial modulus (same as elastic modulus, same as Young's modulus) was 5.5 GPa, +/−1 st.dev=4.4-6.6 GPa.

As shown in FIG. 4C, a set of 4 fibers was tested for tensile mechanical properties using an instrument including a linear actuator and calibrated load cell. Fibers were pulled at 1% per second strain rate until failure. Fiber diameters were measured with light microscopy at 20× magnification using image processing software. The mean diameter was 8.9 um, +/−1 st.dev=6.9-11.0 um. The mean max tensile stress was 93.2 MPa, +/−1 st.dev=81.4-105.0 MPa. The mean max strain was 128.9%, +/−1 st.dev=84.0-173.8%. The mean yield stress was 83.3 MPa, +/−1 st.dev=64.9-101.7 MPa. The mean initial modulus (same as elastic modulus, same as Young's modulus) was 2.6 GPa, +/−1 st.dev=1.5-3.8 GPa.

FIG. 4D shows a stress strain curve of fibers of the present disclosure in which maximum tensile stress is greater than 100 MPa, maximum tensile stress is from 111 MPa to 130 MPa, initial elastic modulus (i.e. Young's modulus) is from 6 GPa to 7.1 GPa, maximum strain (i.e. extensibility) is from 18% to 111%, and the yield stress is from 107 MPa to 112 MPa. The ultimate tensile stress is also greater than 100 MPa for one of the fibers in this figure.

While not wishing to be bound by theory, the structural properties of the proteins within the spider silk are theorized to be related to fiber mechanical properties. Crystalline regions in a fiber have been linked with the tensile strength of a fiber, while the amorphous regions have been linked to the extensibility of a fiber. The major ampullate (MA) silks tend to have higher strengths and less extensibility than e flagelliform silks, and likewise the MA silks have higher volume fraction of crystalline regions compared with flagelliform silks. Furthermore, theoretical models based on the molecular dynamics of crystalline and amorphous regions of spider silk proteins, support the assertion that the crystalline regions have been linked with the tensile strength of a fiber, while the amorphous regions have been linked to the extensibility of a fiber. Additionally, the theoretical modeling supports the importance of the secondary, tertiary and quaternary structure on the mechanical properties of recombinant protein fibers. For instance, both the assembly of nano-crystal domains in a random, parallel and serial spatial distributions, and the strength of the interaction forces between entangled chains within the amorphous regions, and between the amorphous regions and the nano-crystalline regions, influenced the theoretical mechanical properties of the resulting fibers.

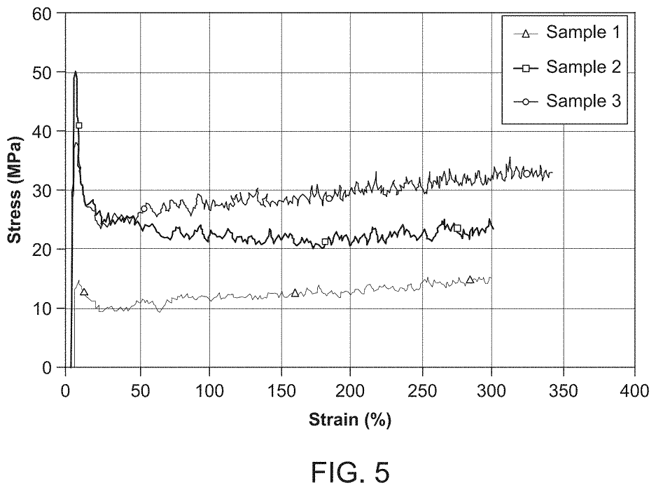

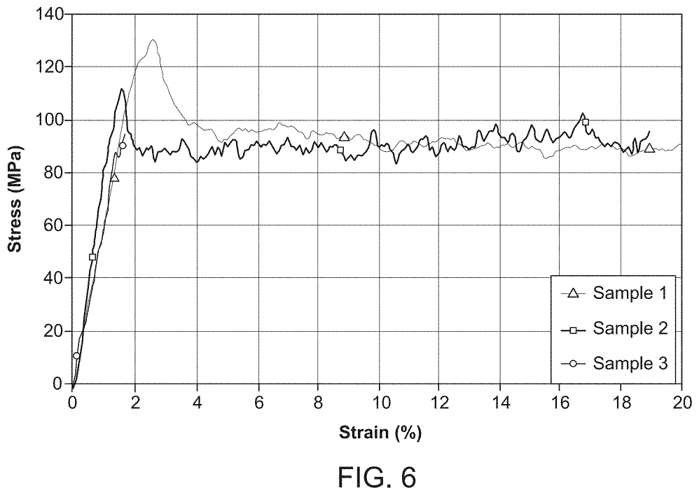

A set of the fibers described herein was tested for tensile mechanical properties using an instrument including a linear actuator and calibrated load cell. Fibers were pulled at 1% per second strain rate until failure. Fiber diameters were measured with light microscopy at 20× magnification using image processing software. The mean maximum stress ranged from 24-172 MPa. The mean maximum strain ranged from 2-342% (see FIG. 5, for example). The mean initial modulus ranged from 1617-7040 MPa (see FIG. 6). The average toughness of three fibers was measured at 0.5 MJ m−3 (standard deviation of 0.2), 20 MJ m−3 (standard deviation of 0.9), and 59.2 MJ m−3 (standard deviation of 8.9). The diameters ranged from 4.48-12.7 μm. Some of the fibers cross-sections were processed to be circular with smooth surfaces, some with a hollow core, and some with rough corrugated surfaces described as corrugated (FIGS. 2 and 3, respectively).

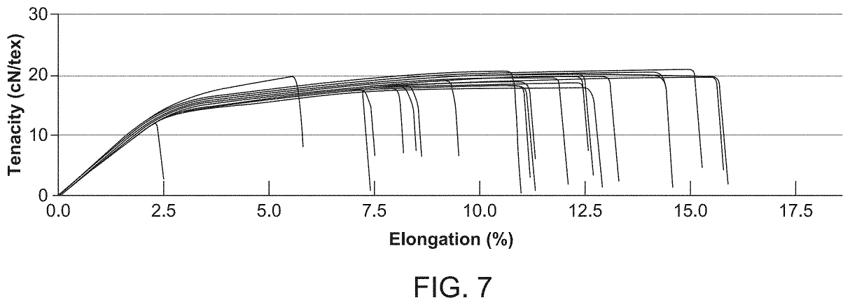

FIG. 7 shows stress strain curves of 23 fibers of the present disclosure, which includes fibers with maximum tensile stress greater than 20 cN/tex, and the average of the maximum tensile stresses of the 23 fibers is about 18.6 cN/tex. The maximum tensile stress ranges from about 17 to 21 cN/tex, and the standard deviation of the maximum tensile stress in this example is about 1.0 cN/tex. The average initial elastic modulus (i.e. Young's modulus) of the 23 fibers is about 575 cN/tex, and the standard deviation in this example is about 6.7 cN/tex. The average maximum elongation of the 23 fibers is about 10.2%, and the standard deviation in this example is about 3.6%. The average work of rupture (a measure of toughness) of the 23 fibers is about 0.92 cN*cm, and the standard deviation in this example is about 0.43 cN*cm. The average linear density of the 23 fibers is about 3.1 dtex, and the standard deviation in this example is about 0.11 dtex.

### Additional Considerations

The foregoing description of the embodiments of the disclosure has been presented for the purpose of illustration; it is not intended to be exhaustive or to limit the claims to the precise forms disclosed. Persons skilled in the relevant appreciate that many modifications and variations are possible in light of the above disclosure.

The language used in the specification has been principally selected for readability and instructional purposes, and it may not have been selected to delineate or circumscribe the inventive subject matter. It is therefore intended that the scope of the disclosure be limited not by this detailed description, but rather by any claims that issue on an application based hereon. Accordingly, the disclosure of the embodiments is intended to be illustrative, but not limiting, of the scope of the invention, which is set forth in the following claims.

1. A proteinaceous block copolymer fiber, wherein the block copolymer comprises:
at least two occurrences of a repeat unit, the repeat unit comprising:
more than 150 amino acid residues and having a molecular weight of at least 10 kDal;
an alanine-rich region with 6 or more consecutive amino acids, comprising an alanine content of at least 80%;
a glycine-rich region with 12 or more consecutive amino acids, comprising a glycine content of at least 40% and an alanine content of less than 30%; and
wherein the fiber comprises at least one property selected from the group consisting of a modulus of elasticity greater than 550 cN/tex, an extensibility of at least 10% and an ultimate tensile strength of at least 15 cN/tex.

2. The fiber of claim 1, wherein the repeat unit comprises from 150 to 1000 amino acid residues.

3. The fiber of claim 1, wherein the repeat unit has a molecular weight from 10 kDal to 100 kDal.

4. The fiber of claim 1, wherein the repeat comprises from 2 to 20 alanine-rich regions.

5. The fiber of claim 1, wherein each alanine-rich region comprises from 6 to 20 consecutive amino acids, comprising an alanine content from 80% to 100%.

6. The fiber of claim 1, wherein the repeat unit comprises from 2 to 20 glycine-rich regions.

7. The fiber of any of claim 1, wherein each glycine-rich region comprises from 12 to 150 consecutive amino acids, comprising a glycine content from 40% to 80%.

8. The fiber of claim 1, wherein the repeat unit comprises 315 amino acid residues, 6 alanine-rich regions, and 6 glycine-rich regions,
wherein the alanine-rich regions comprise from 7 to 9 consecutive amino acids, and alanine content of 100%, and
wherein the glycine-rich regions comprise from 30 to 70 consecutive amino acids, and glycine content from 40 to 55%.

9. The fiber of claim 1, wherein the modulus of elasticity is from 550 cN/tex to 1000 cN/tex.

10. The fiber of claim 1, wherein the extensibility is from 10% to 20%.

11. The fiber of claim 1, wherein the ultimate tensile strength is from 15 cN/tex to 100 cN/tex.

12.-14. (canceled)

15. The fiber of claim 1, wherein the modulus of elasticity is greater than 550 cN/tex, the extensibility is at least 10%, and ultimate tensile strength is at least 15 cN/tex.

16. The fiber of claim 1, wherein each repeat unit has at least 95% sequence identity to a sequence that comprises from 2 to 20 quasi-repeat units, each quasi-repeat unit having a composition comprising {GGY-[GPG-X₁]ₙ₁-GPS-(A)ₙ₂}, wherein for each quasi-repeat unit:
X₁ is independently selected from the group consisting of SGGQQ, GAGQQ, GQGPY, AGQQ, and SQ; and
n1 is from 4 to 8, and n2 is from 6 to 10.

17. The fiber of claim 16, wherein n1 is from 4 to 5 for at least half of the quasi-repeat units.

18. The fiber of claim 16, wherein n2 is from 5 to 8 for at least half of the quasi-repeat units.

19. The fiber of claim 1, wherein the quasi repeat unit has at least 95% sequence identity to a MaSp2 dragline silk protein sequence.

20. The fiber of claim 1, wherein:
the alanine-rich regions form a plurality of nanocrystalline beta-sheets; and
the glycine-rich regions form a plurality of beta-turn structures.

21. The fiber of claim 1, wherein the repeat unit of the block copolymer comprises SEQ ID NO: 1.

22. A method of synthesizing a proteinaceous block copolymer fiber, the method comprising:
expressing a block copolymer polypeptide wherein the block copolymer comprises at least two repeat units, each repeat unit comprising:
more than 150 amino acid residues and having a molecular weight of at least 20 kDal;
an alanine-rich region with 6 or more consecutive amino acids, comprising an alanine content of at least 80%; and
a glycine-rich region with 12 or more consecutive amino acids, comprising a glycine content of at least 40% and an alanine content of less than 30%;
formulating a spin dope comprising the expressed polypeptide and at least one solvent; and
extruding the spin dope through a spinneret and through at least one coagulation bath to form the fiber, wherein
the fiber comprises a property selected from the group consisting of a modulus of elasticity greater than 400 cN/tex, an extensibility of at least 10% and an ultimate tensile strength of at least 15 cN/tex.

23.-45. (canceled)
